SEQUENCE LISTING (34 sequences)

SEQ ID NO: 1 - protein, 685 aa, Homo sapiens
MDVCVRLALWLLWGLLLHQGQSLSHSHSEKATGTSSGANSEESTAAEFCRIDKPLCHSEDEKLSFEAVRNIHKLMDDDANGDVDVEESDEFLREDLNYHDPTVKHSTFHGEDKLISVEDLWKAWKSSEVYNWTVDEVVQWLITYVELPQYEETFRKLQLSGHAMPRLAVTNTTMTGTVLKMTDRSHRQKLQLKALDTVLFGPPLLTRHNHLKDFMLVVSIVIGVGGCWFAYIQNRYSKEHMKKMMKDLEGLHRAEQSLHDLQERLHKAQEEHRTVEVEKVHLEKKLRDEINLAKQEAQRLKELREGTENERSRQKYAEEELEQVREALRKAEKELESHSSWYAPEALQKWLQLTHEVEVQYYNIKKQNAEKQLLVAKEGAEKIKKKRNTLFGTFHVAHSSSLDDVDHKILTAKQALSEVTAALRERLHRWQQIEILCGFQIVNNPGIHSLVAALNIDPSWMGSTRPNPAHFIMTDDVDDMDEEIVSPLSMQSPSLQSSVRQRLTEPQHGLGSQRDLTHSDSESSLHMSDRQRVAPKPPQMSRAADEALNAMTSNGSHRLIEGVHPGSLVEKLPDSPALAKKALLALNHGLDKAHSLMELSPSAPPGGSPHLDSSRSHSPSSPDPDTPSPVGDSRALQASRNTRIPHLAGKKAVAEEDNGSIGEETDSSPGRKKFPLKIFKKPLKK

SEQ ID NO: 2 - protein, 685 aa, Pan troglodytes; identical to SEQ ID NO: 1

SEQ ID NO: 3 - protein, 685 aa, Canis lupus familiaris
MDVCARVALWLLWGLLLHHGQSFSHSHSEKATGAGSGATSEESTAAEFCRIDKPLCHSEDEKLSFDAVRSIHKLMDDDANGDVDVEESDEFLREDLNYHDPTVKHSTFHGEDKLISVEDLWKAWKSSEVYNWTVDEVVQWLITYVELPQYEETFRKLQLSGHAMPRLAVTNTTMTGTVLKMTDRSHRQKLQLKALDTVLFGPPLLTRHNHLKDFMLVVSIVIGVGGCWFAYIQNRYSKEHMKKMMKDLEGLHRAEQSLHDLQERLHKAQEEHRTVEVEKVHLEKKLRDEINLAKQEAQRLKELREGTENERSRQKYAEEELEQVREALRKAEKELESHSSWYAPEALQKWLQLTHEVEVQYYNIKKQNAEKQLLVAKEGAEKIKKKRNTLFGTFHVAHSSSLDDVDHKILTAKQALSEVTAALRERLHRWQQIEILCGFQIVNNPGIHSLVAALNIDPSWMGSTRPNPAHFIMTDDVDDMDEEIVSPLSMQSPTLQCSVRQRLTEPQHGLGSQRDLTLSDSESSLHMSDRQRLVPKPPQMVRAVDEALSAMTSNGSHRLIEGVHPGSLVEKLPDSPALAKKALLALNHGLDKAHSLMELSPSAPPGGSPLLDSSRSHSSSSPDPDMPSPIGDSRAQQASRNTRIPHLAGKKAVADEDNGSIGEETDSSPGRKKFPLKIFKKPLKK

SEQ ID NO: 4 - protein, 683 aa, Bos taurus
MDVCARLALWLLWGLLLHHGQSLSQSHSEKATGSGANSEESTAAEFCRIDKPLCHSEDEKLSFDAVRSIHKLMDDDANGDVDVEESDEFLREDLNYHDPTVKHSTFHGEDKLISVEDLWKAWKSSEVYNWTVDEVVQWLITYVELPQYEETFRKLQLSGHAMPRLAVTNTTMTGTVLKMTDRSHRQKLQLKALDTVLFGPPLLTRHNHLKDFMLVVSIVIGVGGCWFAYIQNRYSKEHMKKMMKDLEGLHRAEQSLHDLQERLHKAQEEHRTVEVEKVHLEKKLRDEINLAKQEAQRLKELREGTENERSRQKYAEEELEQVREALRKAEKELESHSSWYAPEALQKWLQLTHEVEVQYYNIKKQNAEKQLLVAKEGAEKIKKKRNTLFGTFHVAHSSSLDDVDHKILTAKQALSEVTAALRERLHRWQQIEILCGFQIVNNPGIHSLVAALNIDPSWMGSTRPNPAHFIMTDDVDDMDEEIVSPLSMQSPSLQSSVRQRLTEPQHGLGSQRDLTHSDSESSLHMSDRQRLAPKPPQMIRAADEALSAMTSNGSHRLIEGAHPGSLVEKLPDSPALAKKALLALNHGLDKAHSLMELSSPALPSGSPHLDSSRSHSPSPPDPDTPSPAGDSRALQASRNTRIPHLAGKKAAAEEDNGSIGEETDSSPGRKKFPLKIFKKPLKK

SEQ ID NO: 5 - protein, 685 aa, Mus musculus
MDVCARLALWLLWGLLLHQGQSLSHSHSEKNTGASSGATSEESTEAEFCRIDKPLCHSEDEKLSFEAVRNIHKLMDDDANGDVDVEESDEFLREDLNYHDPTVKHSTFHGEDKLISVEDLWKAWKSSEVYNWTVDEVIQWLITYVELPQYEETFRKLQLTGHAMPRLAVTNTTMTGTVLKMTDRSHRQKLQLKALDTVLFGPPLLTRHNHLKDFMLVVSIVIGVGGCWFAYIQNRYSKEHMKKMMKDLEGLHRAEQSLHDLQERLHKAQEEHRTVEVEKVHLEKKLRDEINLAKQEAQRLKELREGTENERSRQKYAEEELEQVREALRKAEKELESHSSWYAPEALQKWLQLTHEVEVQYYNIKKQNAERQLLVAKEGAEKIKKKRNTLFGTFHVAHSSSLDDVDHKILTAKQALSEVTAALRERLHRWQQIEILCGFQIVNNPGIHSLVAALNIDPSWMGSTRPNPAHFIMTDDVDDMDEEIVSPLSMQSPSLQSSVRQRLTEPQLGLGSQRDLTHSDSESSLHMSDRQRVAPKPPQMGRAADEALNAMPSNGSHRLIEGVHPGSLVEKLPDSPALAKKTFMALNHGLDKAHSLMELNPSVPPGGSPLLDSSHSLSPSSPDPDTPSPVGDNRALQGSRNTRIPHLAGKKAMAEEDNGSIGEETDSSPGRKKFPLKIFKKPLKK

SEQ ID NO: 6 - protein, 685 aa, Rattus norvegicus
MDVCARLALWLLWGLLLHQGQSLSHSHSEKNTGASSGATSEESTEAEFCRIDKPLCHSEDEKLSFEAVRNIHKLMDDDANGDVDVEESDEFLREDLNYHDPTVKHSTFHGEDKLISVEDLWKAWKASEVYNWTVDEVIQWLITYVELPQYEETFRKLQLTGHAMPRLAVTNTTMTGTVLKMTDRSHRQKLQLKALDTVLFGPPLLTRHNHLKDFMLVVSIVIGVGGCWFAYIQNRYSKEHMKKMMKDLEGLHRAEQSLHDLQERLHKAQEEHRTVEVEKVHLEKKLRDEINLAKQEAQRLKELREGTENERSRQKYAEEELEQVREALRKAEKELESHSSWYAPEALQKWLQLTHEVEVQYYNIKKQNAERQLLVAKEGAEKIKKKRNTLFGTFHVAHSSSLDDVDHKILTAKQALSEVTAALRERLHRWQQIEILCGFQIVNNPGIHSLVAALNIDPSWMGSTRPNPAHFIMTDDVDDMDEEIVSPLSMQSPSLQSSVRQRLTEPQHGLGSQRDLTHSDSESSLHTSDRQRVAPKPPQMGRAADEALNATSSNGSHRLIEGVHPGSLVEKLPDSPALAKKTILALNHGLDKAHSLMELNPSVPPGGSPLLDSSHSHSPSSPDPDTPSPVGDSRALQGSRNTRIPHLAGKKAMAEEDNGSIGEETDSSPGRKKFPLKIFKKPLKK

SEQ ID NO: 7 - protein, 676 aa, Danio rerio
MEFSGLVTFWIACICFLLQCRADKLNPVNTDPLPADNGVSELCRIDEPLCQDENAILSFEAIRSIHKQMDDDDNGNVDVLETDGFLREDLNYHDPKGKHNTFHGDDQFISVEDLWNAWKSSEVYNWTVDEVVKWLIDYVELTQYVEAIKKLNFSGTAMPRLAVKNNTLTQTVLKILDRSHVQKLQLKALDTVLFGAPMMNRHNHLKDFMLVVSIVIGMGGCWFAYIQNRHSKDHMKKMMKDLDSLQRAEQSLHDLQQKLQIAQEEHRSVEVEKVNLEQKLRDEINTAKQEAQRLKELREGTENELSRQKYAEEELEQVRMALKKAEKELESRSSWSPPEALQKWLQLTHEVEVQYYNIKKQNAERQLQVAKEGAEKIKKKRNTLFGTFHVAHSSSLDDVDHKILAAKQALGEVTAALRERLHRWQQIELLTGFTLVHNPGLPSLASALNLDPSFMGGRGTPQHFMSDDMDDMDEDIVPPGTLQSPSMMSLRQRHIDPQMALGSQRDLNRSDSDSSLCISQTGEQLRLSYSSKGFPVKPTSLLHGPHSRSEEGAQSHTHNGGNRVHDGGPSPDGVPDSPILMKKLYGIEKSASMSEIHGSQAAMSMSESSRSLSPNSTEPDTPSPTGLTGGKAGNRIPQISSKKSPLEEDSGSTGEDTDSAASRKKHTFKIFKKQKK

SEQ ID NO: 8 - protein, 570 aa, Drosophila melanogaster
MRKNTIWNYSLIFFCCVLKSISTLDHGPHTVSVDSNRHNTQHQYKQNPNVASQRHSSHESGQSLHNSQSEHVTHIAASHAGSGGEHSTHLAQNLHRSSYNLLSEAMSQAVSNEFSSMGSGSADGACAADDFDCYSGSVQDRFGMEAIASLHRQLDDDDNGNIDLSESDDFLREELKYDSGYEKRQKAFHFNDDMHISVKELWEAWLRSEVHNWTIEQTTDWLAQSVQLPQYVDLFKLHKVTGAALPRLAVNNLQYVGNVLGIKDPIHKQKISLKAMDVVLFGPPRETGTRWKDYILVTLLLSAIIGCWYAYQQNKNAKRHLRRMAQDMEGLQRAEQSLQEMQKELERARMEQENVATEKLDLERRLKEAPTLSSSNSDLEVQQLKKEIEMLRNELSRAEFELVDNCWSPPPQLQSWLQYTYELESKNHQKKRTSAEKQLQSAREACEKLRKKRSSLVGAFVSTHGKSIDDVDRSIVEARNALGDVTNELQERLHRWKQIETCLGLNIVNNNGLPYLENVLYGRNGGLQSSMGMSSTKGSRARITNSTEDLDDESIQGKLNFENFSLLATE

SEQ ID NO: 9 - protein, 476 aa, Anopheles gambiae
RNSYTVLSQAMSQAVHHEFGIGAMAGDAGSACTIDDIDCLAHHDQLGMEAIRSLHQQLDDDDNGDIDLSESDDFLREELKYDSGYEKRHKAFHFNDDMHISVKELWEAWLRSEVHNWTVDQTTEWLAQSVQLPQYVPLFRLHKVTGKVLPRLAVNNMHYVSNVLGIKDPIHKQKIALKAMDAVLFGPPRETGTRWKDLLLVTLLLTAIIGSWYAYHQNKSAKIHIRRMAKDVEGLLKAEVALKEMQKELEQARIEQENVGKEKMDLERRLREAPTLSSSSSDLELQQLKQEIEVLRSELNRAEVEIHDHCWTPPQGLQSWLQLTYELENKHHIRKRIMAEKQLEQAREACEKLRKKRSSLVGAFVSTHGKSIDDVDRSIVEARNALNDVTNDLQERMHRWKQIETMLGFGIVNNSGIAHLENLLYNRNGVAGKTYRCKHPTAAVMSRLSSSQDDLDDDSVQGKPISFDNFSMFSSE

SEQ ID NO: 10 - protein, 510 aa, Caenorhabditis elegans
MGRVSWIIALYLTINVVIVVNGDRVTRNVEVTAEEEKIRDKLGYEAIRDIHRDMDDDHSGSIDRNESTGFMKEDMQMRGSERTRRENKFHGDDDAITVDDLWEAWFESIERTWTNERLVEWLINDVNLPSIVEAVKAKKIDGKILPRFASPNSDFLNKELGIKSSVYRQKLRLNSLDVVLFGYKDNNNRTKDILLAFLALLLTSLIFLYVRQKQKAQQKVNELSNKLTELKCMETEFEDVQKMLNDERSKRSISDGVVNHTEMENLRVQLEEAERRLEANSNGSQAPLALQPLLRRTCENEMAFLEKQRQDCFKEMKEAIEMVDRLQKKQGSVLSSLKLATGAASTSDQVDSKIFALKSRMEKIHTLTRETQERWLQIESLCGFPLLYLNGSSSSGSISNAHSNPNAVNSNFVKKVSPPIPPSQQTANLRFVPTEQSDSIHSEDTSPIVEDVAISRSLTQDLAEADMQSIVSGSTNGSGSVAALKKRKGIFPKLFRRNTSKSSSLGGTSN

SEQ ID NO: 11 - protein, 749 aa, Homo sapiens
MSFIDPYQHIIVEHQYSHKFTVVVLRATKVTKGAFGDMLDTPDPYVELFISTTPDSRKRTRHFNNDINPVWNETFEFILDPNQENVLEITLMDANYVMDETLGTATFTVSSMKVGEKKEVPFIFNQVTEMVLEMSLEVCSCPDLRFSMALCDQEKTFRQQRKEHIRESMKKLLGPKNSEGLHSARDVPVVAILGSGGGFRAMVGFSGVMKALYESGILDCATYVAGLSGSTWYMSTLYSHPDFPEKGPEEINEELMKNVSHNPLLLLTPQKVKRYVESLWKKKSSGQPVTFTDIFGMLIGETLIHNRMNTTLSSLKEKVNTAQCPLPLFTCLHVKPDVSELMFADWVEFSPYEIGMAKYGTFMAPDLFGSKFFMGTVVKKYEENPLHFLMGVWGSAFSILFNRVLGVSGSQSRGSTMEEELENITTKHIVSNDSSDSDDESHEPKGTENEDAGSDYQSDNQASWIHRMIMALVSDSALFNTREGRAGKVHNFMLGLNLNTSYPLSPLSDFATQDSFDDDELDAAVADPDEFERIYEPLDVKSKKIHVVDSGLTFNLPYPLILRPQRGVDLIISFDFSARPSDSSPPFKELLLAEKWAKMNKLPFPKIDPYVFDREGLKECYVFKPKNPDMEKDCPTIIHFVLANINFRKYKAPGVPRETEEEKEIADFDIFDDPESPFSTFNFQYPNQAFKRLHDLMHFNTLNNIDVIKEAMVESIEYRRQNPSRCSVSLSNVEARRFFNKEFLSKPKA

SEQ ID NO: 12 - protein, 749 aa, Pan troglodytes; identical to SEQ ID NO: 11

SEQ ID NO: 13 - protein, 749 aa, Canis lupus familiaris
MSFIDPYQHIIVEHQYSHKFTVVVLRATKVTKGAFGDMLDTPDPYVELFISSTPDSRKRTRHFNNDINPVWNETFEFILDPNQENVLEVTLMDANYVMDETLGTATFPISSMRVGDKKEVPFIFNQVTEMFLEMSLEVCSSPDLRFSMALCDQEKAFRQQRKENIKENMKKLLGPENSEGLCSTRDVPVVAILGSGGGFRAMVGFSGVIKALYESGILDCATYVAGLSGSTWYMSTLYSHPDFPEKGPEEINEELMKNVSYNPLLLLTPQKVKRYVESLWKKKSSGQPVTFTDIFGMLIGETLIHNRMNTTLSSLKEKVSSGQCPLPLFTCLHVKPDVSELMFADWVEFSPYEIGMAKYGTFMAPDLFGSKFFMGTVVKKYEENPLHFLMGVWGSAFSILFNRVLGVSGSQNKGSTMEEELENITAEHIVSNDSSDSDDETQEPKGTENEDAKREYHNDNQASWVHRMIMALVSDSTLFNTREGRAGKVHNFMLGLNLNTSYPMSPLKDLMTQESLDEDELDMAVADPDEFEQIYEPLDVKSKKIHVVDSGLTFNLPYPLILRPQRGVDLIISFDFSARPSDSSPPFKELLLAEKWAKMNKLPFPKIDPHVFDREGLKECYVFKPKNPDMEKDCPTIIHFVLANINFRKYKAPGVPRETKEEKEIADFDIFDDPESPFSTFNFQYPNQAFKRLHDLMHFNTLNNIDVIKNAIVESIEYRRQNPSRCSVSLSNVEARRFFNKEFLSKPAA

SEQ ID NO: 14 - protein, 749 aa, Bos taurus
MSFIDPYQHIIVEHHYSHKFTVVVLRATKVTKGTFGDMLDTPDPYVELFISSTPDSRKRTRHFNNDINPVWNETFEFILDPNQENILEITLMDANYVMDETLGTTTFPISSMKVGEKKQVPFIFNQVTEMILEMSLEVCSSPDLRFSMALCDQEKAFRQQRKENIKENMKKLLGPKNSEGLHSTRDVPVVAILGSGGGFRAMVGFSGVMKALYESGILDCATYIAGLSGSTWYMSTLYSHPDFPEKGPEEINKELMKNVSHNPLLLLTPQKIKRYVESLWRKKSSGQPVTFTDIFGMLIGETLIHNRMNTTLSSLKEKVNTGQCPLPLFTCLHVKPDVSELMFADWVEFSPFEIGMAKYGTFMAPDLFGSKFFMGTVVKKYEENPLHFLMGVWGSAFSILFNRVLGVSGSQSKGSTMEEELENITAKHIVSNDSSDSDDESQGPKGTEHEEAEREYQNDNQASWVQRMLMALVSDSALFNTREGRAGKVHNFMLGLNLNTSYPMSPLRDFTMQESLDEDELDAAVADPDEFEQIYEPLDVKSKKIHVVDSGLTFNLPYPLILRPQRGVDLIISFDFSARPGDSSPPFKELLLAEKWAKMNKLPFPKIDPYVFDREGLKECYVFKPKNPDVEKDCPTIIHFVLANINFRKYKAPGVPRETNEEKEIADFDIFDDPESPFSTFNFQYPNQAFKRLHDLMYFNTLNNIDVIKNAIVESIEYRRQNPSRCSVSLSSVEARRFFNKEFLSKPTA

SEQ ID NO: 15 - protein, 748 aa, Mus musculus
MSFIDPYQHIIVEHQYSHKFTVVVLRATKVTKGTFGDMLDTPDPYVELFISTTPDSRKRTRHFNNDINPVWNETFEFILDPNQENVLEITLMDANYVMDETLGTATFPVSSMKVGEKKEVPFIFNQVTEMILEMSLEVCSCPDLRFSMALCDQEKTFRQQRKENIKENMKKLLGPKKSEGLYSTRDVPVVAILGSGGGFRAMVGFSGVMKALYESGILDCATYIAGLSGSTWYMSTLYSHPDFPEKGPEEINEELMKNVSHNPLLLLTPQKVKRYVESLWKKKSSGQPVTFTDIFGMLIGETLIQNRMSMTLSSLKEKVNAARCPLPLFTCLHVKPDVSELMFADWVEFSPYEIGMAKYGTFMAPDLFGSKFFMGTVVKKYEENPLHFLMGVWGSAFSILFNRVLGVSGSQNKGSTMEEELENITAKHIVSNDSSDSDDEAQGPKGTENEEAEKEYQSDNQASWVHRMLMALVSDSALFNTREGRAGKVHNFMLGLNLNTSYPLSPLRDFSSQDSFDDELDAAVADPDEFERIYEPLDVKSKKIHVVDSGLTFNLPYPLILRPQRGVDLIISFDFSARPSDTSPPFKELLLAEKWAKMNKLPFPKIDPYVFDREGLKECYVFKPKNPDVEKDCPTIIHFVLANINFRKYKAPGVLRETKEEKEIADFDIFDDPESPFSTFNFQYPNQAFKRLHDLMYFNTLNNIDVIKDAIVESIEYRRQNPSRCSVSLSNVEARKFFNKEFLSKPTV

SEQ ID NO: 16 - protein, 752 aa, Rattus norvegicus
MSFIDPYQHIIVEHQYSHKFTVVVLRATKVTKGTFGDMLDTPDPYVELFISTTPDSRKRTRHFNNDINPVWNETFEFILDPNQENVLEITLMDANYVMDETLGTATFPVSSMKVGEKKEVPFIFNQVTEMILEMSLEVSSCPDLRFSMALCDQEKTFRQQRKENIKENMKKLLGPKKSEGLYSTRDVPVVAILGSGGGFRAMVGFSGVMKALYESGILDCATYVAGLSGSTWYMSTLYSHPDFPEKGPEEINEELMKNVSHNPLLLLTPQKVKRYVESLWKKKSSGLPVTFTDIFGMLIGETLIQNRIVPTLSSLKEKVSAARCPLPLFTCLHVKPDVSELMFADWVEFSPYEIGMAKYGTFMTPDLFGSKFFMGTVVKKYEENPLHFLMGVWGSAFSILFNRVLGVSGLQNKGSTMEEELENITAKHIVSNDSSDSDDEAQGPKGTENEDAEREYQNDNQASWVHRMLMALVSDSALFNTREGRAGKVHNFMLGLNLNTSYPLSPLRDFSPQDSFDDDELDAAVADPDEFERIYEPLDVKSKKIHVVDSGLTFNLPYPLILRPQRGVDLIISFDFSARPSDTSPPFKELLLAEKWAKMNKLPFPKIDPYVFDREGLKECYVFKPKNPDVEKDCTTIIHFVLANINFRKYKAPGVLRETKEEKEIADFDIFDDPESPFSTFNFQYPNQAFKRLHDLMYFNTLNNIDVIKDAIVESIEYRRQNPSRCSVSLSNVEARKFFNKEFLSKPTAESI

SEQ ID NO: 17 - protein, 748 aa, Gallus gallus
MSFIDPYQHIVVEHQYSHVFTVTVRKATNVTKGAIGDMLDTPDPYVELFIPSAPDCRKRTKHFNNDVNPVWNETFEFILDPNQDNVLEVTLMDANYVMDETLGMATFPISSLKLGEKKEVQLTFNNVTEMTLELSLEVCSSTDLRFSMALCDEEKKFRQQRKDNIMQSMKSFLGEENSKNLTTSRDVPVIAVLGSGGGFRAMVGFAGVMKALYESGVLDCATYIAGLSGSTWYMSTLYSHPDFPEKGPKEINQELMNSVSHNPLLLLTPQKVKRYIEALWNKKSSGQPVTFTDIFGMLIGETLIHNRMDTTLSDMKEKVSEAQCALPLFTCLHVKPDVSELMFADWVEFSPYEIGMAKYGTFMSPDLFGSKFFMGTVVKKYSENPLHFLMGVWGSAFSILFNRVLGVSNSQNKGPTMEEELENIRLKHLVSNDSSDSEDESQHPKGTENSEANEEYQNSSQESWVQRMLMALVGDSALFNTREGRAGKVHNFMLGLNLNSCYPLSPLADLLTQESVEEDELDAAVADPDEFERIYEPLDVKSKKIHIVDSGLTFNLPYPLILRPQRGVDLIISFDFSARPSDSSPPFKEILLAEKWAKMNKLPFPKIDPNVFDREGLKECYVFKPKDTSSEKDCPTIIHFVLANINFRKYKAPGLPRESKEEKDFADFDIFDDPNTPFSTFNFQYPNEAFKRLHDLMEFNTLNNLDVIKQAMMESIEYRKENPSRCSVSLSSVEARRFFNKNNLNNHT

SEQ ID NO: 18 - protein, 741 aa, Danio rerio
MSNIIVEHQYSHRLKLTVVRAENVTKGAFGDLLDTPDPYVELSVPTTPESRKRTRHINNDINPKWNETFEFILDPNQSNVLEVTLMDANYVMDETLGTAKYSLSKLKVAQMEHVTLSIGKTTKVFLDLLLEVCASTDLRFSMTLCDQEKLFMQTRRDRVMLSIKKLLKMENPRFLPSSPREVPTIAILGSGGGFRAMVGFSGVMKALYESGVFDCATYVAGLSGSTWYMSMLYSHPEFPAKGPGDINKELMNRVSNNPLKLLLPQNINRYVKALWKKKSAGQPVTFTDIFGMLIGETLIPGRMNIKLSSLKGKINEGQSPLPLFTCLHVKPDVSELMFADWVEFSPYEIGMAKYGTFMSPGLFGSKFFMGSVVKQYEENPLHFLMGVWGSAFSILFNRVLGVKETTSSSTMEEELEQIKPEHIVGDDSADNEEETQRGGTESADAEDERQRHAQASWVQRMLTSIMGDTTLFTTREGRAGKVHNFMLGLNLNSTLPFSPFSGITHQTSLEEEVDAVTDPDEFERIYEPLDVKSKKIHVVDSGLTFNLPYPLILRCQRGVDLIISFDFSARPSDSSPPFKELLLAEKWARMNKLPFPKIDSKVFDREGLKECYVFKPAKGDKNCPTIIHFVLANINFRNFKAPGVPRDSDKDIEFGDFDIFDEPASPYSTFNFKYNNQAFKRLHDLMEFNTLNNIEVIKEAIKDSILLRRENPARCSVSLSLSEIENKKFLKRDNSIAKRPT

SEQ ID NO: 19 - protein, 697 aa, Danio rerio
MSPKELSETCVMDTPDPYVELFIPTSPESRKKTRHIDNDINPEWNETFEFILDPNQDNVLKLTLMDANYVVDETLGTASYDISKLKVGQTMLVSFLIGKMTNVFLELSLEECLAMDLRFNLDLCPKEKLYRQHRRDKVMLAIKKLLNIEKPSSLPCSPHEVPVIAVLGSGGGFRAMIGFSGVMKALYISGVLDCVTYVAGLSGSTWYMSTLYSHPEFPTKGPEQINQELMNSVSSNPLRLLHPKHITNYIHALWSKKTTGQPVTFTDIFGMLIGETLIPARMDTKLRDLQEKINEAQAPLPLFTCLHVKPDVSELKFADWVEFSPYEIGMAKYGTFMTPDLFGSKFFMGNVVKKHEENPLHFLMGVWGSAFSILFNRILGLTDVTNGSTMEEELEQIKPEHIVGEDSLDNDEDPHKEDEHQASWIQRMVGSLFSDSVLFNTREGRAGKVHNFMLGLNLNNDVPFSPYTETISCTEDEVDAVTDPFEFDRIYEPLDVKSRKIHVVDSGLTFNLPYPLILRPQRGVDLIISFDFSARPSDSSPPFKELLLAEKWARMNKLPFPKIDPKVFDREGLKECYVFKPKKGERNCPTIIHFVLVNIEFRKFKAPGVPRGSDKEREFADFDIFDDPQTPYSTFNFYYSHQAFTQLHDLMEFNTLNNIEVIKEGIKDSILYRKEIPSRCSASLSLSEAQKKKRLKK

SEQ ID NO: 20 - protein, 295 aa, Homo sapiens [sequence omitted]

SEQ ID NO: 21 - protein, 295 aa, Canis lupus familiaris
MKGGEGDAGEQAPLNPEAESPAGSATYREFVHRGYLDLMGASQHSLRALSWRRLYLSRAKLKASSRTSALLSGFAMVAMVEVQLESNHEYPPGLLVAFSACTTVLVAVHLFALMVSTCLLPHIEAVSNIHNLNSVHQSPHQRLHRYVELAWGFSTALGTFLFLAEVVLVGWVKFVPIGTPLATPAPVVPASQVPGTLLPLTTSLSPAPKPSSTPVAPSQAEPAEACPPWQACVGGGAHGPGWQAAMASTAIMVPVGLVFVAFALHFYRSLVAHKTDRHKQELEELSRLQGELQAV

SEQ ID NO: 22 - protein, 291 aa, Bos taurus
MKGGEGDPGEQAPLNPEAESPAGSATYREFVHRGYLDLMGASQHSLRALSWRRLYLSRAKLKASSRTSALLSGFAMVAMVEVQLESKHEYPPGLLVAFSACTTVLVAVHLFALMVSTCLLPHIEAVSNIHNLNSVHQSPHQRLHRYVELAWGFSTALGTFLFLAEVVLVGWVKFVPIGAPLGTSGSVGPASQLPTVATSLSPASKPSSVSVAPSQAEPPEPCPPRQACGSGAAHGPGWQAAMASTAIMVPVGLVFVAFALHFYRSLVAHKTDRHKQELEELSRLQGELQAV

SEQ ID NO: 23 - protein, 290 aa, Mus musculus
MKGGEGDTGEQAPLNPEVDSPAGSATYREFVHRGYLDLMGASQHSLRALSWRRLYLSRAKLKASSRTSALLSGFAMVAMVEVQLENDHEYPPGLLVAFSACTTVLVAVHLFALMVSTCLLPHIEAVSNIHNLNSVHQSPHQRLHRYVELAWGFSTALGTFLFLAEVVLVGWVKFVPIGAPMGKPAPVVPMSQVPPVTVSLSLASNLTPSSASITTSQQPSKACPPRQVCDSAHGPGWQAAMASTAIMVPVGLVFMAFALHFYRSLVAHKTDRHKQELEELSRLQGELQAV

SEQ ID NO: 24 - protein, 290 aa, Rattus norvegicus
MKGGEGDTGEQAPLNPEVDSPAGSATYREFVHRGYLDLMGASQHSLRALSWRRLYLSRAKLKASSRTSALLSGFAMVAMVEVQLESDHEYPPGLLVAFSACTTVLVAVHLFALMVSTCLLPHIEAVSNIHNLNSVHQSPHQRLHRYVELAWGFSTALGTFLFLAEVVLVGWVKFVPIGAPMDKPAPVVPMSQVPPVTVSLSLASNLTPSSASVATTQQPPKACPPRQVCGSAHGPGWQAAMASTAIMVPVGLVFMAFALHFYRSLVAHKTDRHKQELEELSRLQGELQAV

SEQ ID NO: 25 - protein, 301 aa, Homo sapiens
MHPEPAPPPSRSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKKQPGQPRPTSKPPASGAAANVSTSGITPGQAAAIASTTIMVPFGLIFIVFAVHFYRSLVSHKTDRQFQELNELAEFARLQDQLDHRGDHPLTPGSHYA

SEQ ID NO: 26 - protein, 301 aa, Pan troglodytes
MHPEPAPPPSGSSPELPPSGGSTTSGSRRSRRRSGDGEPPGAPPPPPSAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKKQPGQPRPTSKPPAGGAAANVSTSGITPGQAAAIASTTIMVPFGLIFIVFAVHFYRSLVSHKTDRQFQELNELAEFARLQDQLDHRGDHPLTPGSHYA

SEQ ID NO: 27 - protein, 361 aa, Canis familiaris
MLITPRLRPIAARAEGPGHGSRTLACAQARVRAAPGFLQAARRACEEPQPARAFAPCSMHPEPAPPPNSNSPELPLGGGSTTSGSRRSRRRSGDGEPPGSPPPPAAVTYPDWIGQTYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKKQPGQPRPTSKPPAGSAVASVNGSSTGGITPGQAAAIASTTIMVPFGLVFIVFAVHFYRSLVSHKTDRQFQELNELAEFARLQDQLDHRGDHPLTPSSHYA

SEQ ID NO: 28 - protein, 303 aa, Bos taurus
MHPEPAPPPSSNSPELPLSGGNSTSGSRRSRRRSGDGEPPGSPPPPPPPAVTYPDWIGQSYSEVMSLNEHSMQALSWRKLYLSRAKLKASSRTSALLSGFAMVAMVEVQLDADHDYPPGLLIAFSACTTVLVAVHLFALMISTCILPNIEAVSNVHNLNSVKESPHERMHRHIELAWAFSTVIGTLLFLAEVVLLCWVKFLPLKKQPGQLRPTSKPPAGGAAANVSSTGGITPGQAAAIASTTIMVPFGLIFIVFAVHFYRSLVSHKTDRQFQELNELAEFARLQDQLDHRGDHPLTPGSHYA

SEQ ID NO: 29 - protein, 304 aa, Mus musculus
MHPEPAPPPSHSNPELPVSGGSSTSGSRRSRRRSGDGEPSGAPPLPPPPPAVSYPDWIGQSYSEVMSLNEHSMQALSWRKLYLSRAKLKASSRTSALLSGFAMVAMVEVQLDTDHDYPPGLLIVFSACTTVLVAVHLFALMISTCILPNIEAVSNVHNLNSVKESPHERMHRHIELAWAFSTVIGTLLFLAEVVLLCWVKFLPLKRQAGQPSPTKPPAESVIVANHSDSSGITPGEAAAIASTAIMVPCGLVFIVFAVHFYRSLVSHKTDRQFQELNELAEFARLQDQLDHRGDHSLTPGTHYA

SEQ ID NO: 30 - protein, 304 aa, Rattus norvegicus
MHPEPAPPPNNSNPELPLSGGSSTSGSRRSRRRSGDGEPTGAPPLPPPPAVSYPDWIGQSYSEVMSLNEHSMQALSWRKLYLSRAKLKASSRTSALLSGFAMVAMVEVQLDTDHDYPPGLLIVFSACTTVLVAVHLFALMISTCILPNIEAVSNVHNLNSVKESPHERMHRHIELAWAFSTVIGTLLFLAEVVLLCWVKFLPLKRQAGQPSPTKPPTEPAVVVANSSNNGGITPGEAAAIASTAIMVPCGLVFIVFAVHFYRSLVSHKTDRQFQELNELAEFARLQDQLDHRGDHSLTPGTHYA

SEQ ID NO: 31 - protein, 226 aa, Gallus gallus
MSLNEHSMQALSWRKLYLSRAKLKASSRTSALLSGFAMVAMVEVQLDAEHDYPRGLLIAFSACTTVLVAVHLFALMISTCILPNIEAVSNVHNLNSVKESPHERMHRHIELAWAFSTVIGTLLFLAEVVLLCWVKFLPLKKNPLDPAENSNSSITSGQAAAIASTSIMVPFGLIFIVFAVHFYRSLVSHKTDRQFQELNELAEFAWLQDQLDHRGDAISPAVTHFA

SEQ ID NO: 32 - protein, 225 aa, Danio rerio
MSLNEHSLQALSWRKLYLSRAKLKATSRTSALLSGFAMVAMVECQLEEDHNYPPGLLIAFSACTTVLVAVHLFALMISTCILPNLEAVSNVHNLNSIKESPHERMHRHIELAWAFSTVIGTLLFLAEVVLLCWVKFLPLSQTPVTQKENVTQAPGISEGKAAAIASTTIMVPFALVFIVFAVHFYRSLVSHKTERQHEELEVLAKITQMQNQLDQRGDASSIHFA

SEQ ID NO: 33 - protein, 351 aa, Drosophila melanogaster
MSVWTTANNSGLETPTKSPITSSVPRAARSSAVITTGNHQQHHFQHVVAAAVAAATSVATGHQFQQQFPLHAHPHPQHHSNSPTGSGSNSNNSAGFQRTSISNSLLQFPPPPPPSSQNQAKPRGHHRTASSSMSQSGEDLHSPTYLSWRKLQLSRAKLKASSKTSALLSGFAMVAMVEVQLDHDTNVPPGMLIAFAICTTLLVAVHMLALMISTCILPNIETVCNLHSISLVHESPHERLHWYIETAWAFSTLLGLILFLLEIAILCWVKFYDLSPPAAWSACVVLIPVMIIFMAFAIHFYRSLVSHKYEVTVSGIRELEMLKEQMEQDHLEHHNNIRNNGMNYGASGDIV

SEQ ID NO: 34 - protein, 343 aa, Anopheles gambiae
MFNGTVSVWSTSTAGIDTVLATTLGTAVTPKTFSTGQLHQTGLSHQPNGTGNGNGVVHHHPNVGNGTGLNHAHLHIGSNGNSHSGSIGNNSTHSLTPHGGHGGASSWNNRTSICSTHNKRRSNLSTMSQTGDDLHTPNYLSWRKLQLSRAKLKASSKTSALLSGFAMVAMVEVQLDENTKVPSAMLIAFAVCTTLLVAVHMLALMISTCILPNIETVCNLHSISLVHESPHERLHWYIETAWAFSTLLGLILFLLEIAILCWVKFYDLNTTAAWSACIVLIPVLVIFVAFALHFYRSLMMHKYEVTVSGIRELEILKEQMEQDHFDQQQHHHGILHPSGLQIV

(19) United States
(12) Patent Application Publication
(10) Pub. No.: US 2013/0017198 A1
(43) Pub. Date: Jan. 17, 2013

(54) MANIPULATION OF CALCIUM CHANNELS TO REGULATE AFTER-DEPOLARIZATION EVENTS IN CARDIAC MYOCYTES

(71) Applicants: Paul Wolkowicz, Hoover, AL (US); Jian Huang, Birmingham, AL (US)

(72) Inventors: Paul Wolkowicz, Hoover, AL (US); Jian Huang, Birmingham, AL (US)

(73) Assignee: THE UAB RESEARCH FOUNDATION, Birmingham, AL (US)

(21) Appl. No.: 13/637,213

(22) PCT Filed: Mar. 25, 2011
(86) PCT No.: PCT/US11/29968

§ 371 Date: Sep. 25, 2012

Related U.S. Application Data
(60) Provisional application No. 61/317,583, filed on Mar. 25, 2010.

(51) Int. Cl. A61K 39/395; C12Q 1/44; C12Q 1/02; C08B 37/16; A61K 31/7088; A61K 31/715; C07K 16/00; C07H 21/00; G01N 33/53; C12N 5/077

(52) U.S. Cl. 424/133.1; 424/141.1; 435/7.1; 435/19; 435/29; 435/375; 514/44 R; 514/44 A; 514/59; 530/387.3; 530/388.1; 536/23.1; 536/24.5; 536/103

(57) ABSTRACT

A novel mechanism by which after-depolarization occurs in cardiac myocytes has been discovered, involving calcium influx through the arachidonate-regulated calcium channel (ARCC) and the store-operated calcium channel (SOCC). Because after-depolarization of the myocyte is a major cause of cardiac arrhythmia, this discovery provides new approaches for treating and preventing heart disease. By down-regulating the activity of the ARCC or the SOCC, after-depolarization can be decreased and cardiac arrhythmia can be prevented, reduced, or eliminated. This can be accomplished using pharmaceuticals containing inhibitors of the ARCC or the SOCC, or by genetically modifying cells to reduce ARCC or SOCC activity. In addition, assays are disclosed using the ARCC or SOCC to discover potential anti-arrhythmic agents. Cellular and animal models of arrhythmia are disclosed in which the activity of the ARCC or SOCC is increased to promote after-depolarization and induce arrhythmia.

### BACKGROUND

A. Field of the Disclosure

The present disclosure relates generally to compositions for the modulation of myocyte after-depolarization and associated cardiac arrhythmia. Such compositions, methods of using them, and assays for detecting them are also provided.

B. Background

Cardiac arrhythmia is a leading cause of premature death and disability, annually afflicting over 3 million people and causing over 350,000 deaths in the United States. Despite this huge biomedical burden, no currently used non-invasive therapies effectively suppress arrhythmia. Consequently, current therapy for arrhythmia involves the invasive ablation or destruction of arrhythmic heart muscle. While ablation restores normal heart rhythm, its action is often temporary so that continued suppression of arrhythmia may require multiple ablations during the lifetime of a patient. Thus, a need exists for non-invasive therapies to treat cardiac arrhythmia.

Normally only the sinoatrial node in the right atrium generates the repeated electrical impulses that propagate through heart muscle and stimulate it to contract. The generation of abnormal electrical impulses or the abnormal propagation of electrical impulses produces arrhythmia. As abnoimal electrical impulse generation usually precede abnormal propagation, interdicting abnormal impulse generation is critical to the non-invasive treatment of arrhythmia. After-depolarization is an important mechanism that generates abnormal electrical impulses. Deranged intracellular calcium homeostasis is a leading explanation for after-depolarization, but the root cause is unresolved. Current theories propose that calcium leakage within myocytes activates plasma membrane ion channels that result in after-depolarization.

Specialized cells of the sinoatrial node initiate normal heart rhythm. These cells spontaneously and repeatedly depolarize to produce electrical signals. These normal electrical signals then proceed first through the upper atrial chambers of the heart and then though the electrically-insulated conduction system of the lower, ventricular chambers of the heart. These electrical impulses then impinge on individual heart muscle cells (myocytes), bringing about their depolarization. This normal activity of the sinoatrial node, the conduction system, and the myocardial muscles is responsible for normal heart function.

Myocytes contract in response to depolarization of the electrochemical gradient across the plasma membrane. A sufficiently strong depolarizing impulse causes the opening of sodium channels found in the myocyte plasma membrane that exclusively transport sodium down its charge gradient. The nearly instantaneous opening of sodium channels brings about a rapid influx of a small amount of sodium into the myocyte, causing the negatively charged interior of the cell to become more positive. This results in depolarization. Sodium channels largely enter into a closed state soon after a depolarization event has occurred. The myocyte plasma membrane also contains multiple ion channels that transport potassium out of the cell, each with unique voltage-dependent behaviors. These potassium channels open in depolarized myocytes and bring about re-polarization, restoring the myocyte to its normal, polarized, resting state. A voltage-dependent calcium channel also opens in depolarized myocytes, allowing the entry of a small amount of calcium into myocytes. This small influx of calcium activates the ryanodine receptor channel on the surface of the sarcoplasmic reticulum (SR). The SR normally contains an internal store of calcium. Activation of the ryanodine receptor channel causes large amounts of calcium from the SR calcium store to rapidly enter the cytoplasm. The calcium released binds to and activates myofilaments. This initiates myocyte contraction (“shortening”). The widely accepted model of these events is shown in FIG. 17.

Arrhythmic activity arises either when a heart muscle other than the sinoatrial node produces electrical impulses, or when electrical impulses initiated by the sinoatrial node or by other cells propagate abnormally through heart muscle. The first case is termed abnormal impulse generation, the second abnormal impulse propagation. Arrhythmia that results from abnormal impulse generation is termed “triggered arrhythmia” as a preceding normal action potential is required as a triggering event. Arrhythmia that arises from abnormal impulse propagation is termed “reentrant arrhythmia.” In most cases, abnormal impulse generation precedes abnormal impulse propagation, so that interdicting the generation of abnormal impulses may suppress either type of arrhythmia. The most widely accepted causes of triggered arrhythmia are two types of after-depolarization termed early and late (or delayed) after-depolarization (“EAD” and “DAD,” respectively).

The two recognized types of after-depolarization differ in that whereas an EAD is observed as a depolarization event that occurs during a prolonged period of re-polarization, a DAD event is observed as a depolarization event that occurs immediately after the myocyte has experienced normal depolarization and subsequent re-polarization. In the case of an EAD event, the duration of the depolarization can increase because of an increase in the depolarizing sodium current, or because of a decrease in one or more of the re-polarizing potassium currents. A sufficiently large EAD can propagate through the heart and produce a second, abnormal wave of atrial or ventricular depolarization that closely follows the preceding normal action potential. This results in an arrhythmic heart beat.

During a DAD event, a myocyte that has re-polarized after a normal depolarization event will depolarize spontaneously. This abnormal depolarization will produce ectopic electrical activity that propagates through quiescent myocardium.

One defining characteristic of after-depolarization events is that they require a preceding normal depolarization event. In contrast, “abnormal automaticity” is a type of arrhythmic activity that may either arise from triggered after-depolarization events or arise independently of triggered after-depolarization events. During abnormal automaticity heart muscle produces repeated spontaneous depolarization events that do not depend on normal cardiac electrical activity. That is, abnormal automaticity occurs when non-automatic atrial or ventricular muscle spontaneously and repeatedly depolarizes.

The current hypothesis to explain after-depolarization events states that the ryanodine receptor channel becomes leaky to calcium under certain circumstances. The slow leak of SR calcium activates calcium-dependent ion channels like the sodium-calcium exchanger (NCX), resulting in depolarization. Thus, after-depolarization and triggered activity are thought to be calcium-dependent phenomena that require high levels of SR calcium and abnormal leakage of SR calcium through the ryanodine receptor channel.

### SUMMARY

Because most arrhythmias result from altered intracellular calcium handling, anti-arrhythmic pharmaceuticals aimed at managing the action potential (myocyte depolarization and repolarization) may not affect the primary cellular cause of the arrhythmic event. Few classes of pharmaceuticals effectively prevent or reverse atrial or ventricular arrhythmic activity, and these generally act by modulating the myocardial action potential. Clinical studies have shown that these anti-arrhythmic agents themselves can be pro-arrhythmic, increasing patient mortality and limiting their effectiveness as anti-arrhythmic therapies. Thus, few pharmaceuticals effectively prevent or reverse clinically relevant forms of arrhythmias without significant side-effects on the normal electrical activity of the heart. One likely reason for this paucity of effective anti-arrhythmic pharmaceuticals may be that agents which target the myocyte proteins or the myocyte processes that underlie arrhythmic activity have not yet been identified.

There is a long-felt but unmet need in the art for ways to effectively treat and prevent cardiac arrhythmias. Despite the seriousness of the disease, and the mortality and morbidity associated therewith, the art has failed to develop and implement consistent treatments for therapeutic intervention.

The current disclosure provides inhibitors of arrhythmia comprising an active compound that inhibits the activity of the arachidonate-regulated calcium (ARC) channel and the related store-operated calcium channel (SOCC), pharmaceutical compositions comprising the active compound, uses of the active compound for the treatment and prevention of arrhythmia, and uses of the active compound for the manufacturing of a pharmaceutical for the treatment and prevention of arrhythmia. Methods of treatment and prevention of arrhythmia comprising administration of the active compound are also provided. Methods and uses for preventing and reversing after-depolarization in a cell comprising contacting the cell to the active compound are provided.

The disclosure provides assays for detecting anti-arrhythmic agents comprising contacting a candidate anti-arrhythmic agent to the ARC channel and measuring the activity of the ARC channel. The ARC channel can exist in any of several milieu, including cell-free assays, in vitro cellular assays, cardiac muscle assays, whole-heart assays, and assays in living subjects.

The disclosure provides methods of inducing after-depolarization in a cell, comprising increasing the activity of the ARC channel in the cell. Increasing ARC channel activity can also induce arrhythmia. Cellular and animal models are provided with increased ARC channel activity, as are uses of such models.

### BRIEF DESCRIPTION OF THE DRAWINGS

FIG. 1: The apparent mechanism by which calcium influx activates the ARC channel. PL is membrane phospholipid containing arachidonic acid. AA is liberated arachidonate. ARC is arachidonate-regulated calcium channel.

FIG. 2: The apparent mechanism by which ARC channel hyperactivity causes after-depolarization, and the mechanism by which ARC channel inhibitors such as LOE-908 prevent after-depolarization. I_{Ti} is transient inward or depolarizing current.

FIG. 3: (A) The apparent mechanism by which ARC channel activity causes after-depolarizations, and by which ARC channel hyperactivity causes abnormal automaticity, and the mechanism by which Orai inhibitors such as SKF-96365 can prevent abnormal automaticity. (B) The apparent mechanism by which prolonged ARC channel activation enhances store-operated calcium channel (SOCC) activation. (C) The apparent mechanism through which plasma membrane Stim1 activates ARC channel activity.

FIG. 4: The conventional mechanistic model of after-depolarization.

FIG. 5: Normal left atrial action potential in response to pacing.

FIG. 6: The action potential profile of a left atrial appendage (LAA) experiencing an after-depolarization in response to ATX-II exposure.

FIG. 7: Normal contractions of paced left atrial appendages (7A) and after-contractions of paced left atrial appendages exposed to ATX-II (7B and 7C).

FIG. 8: Abnormal rapid contractions of paced LAA exposed to high concentrations of ATX-II (8A), and abnormal automatic contractions of unpaced LAA exposed to high concentrations of ATX-II (8B). FIG. 9: The suppression of after-contractions by exposure to the CaMKII inhibitor KN-93. In contrast, KN-92 is an inactive structural analog of KN-93, and does not suppress after-contractions.

FIG. 10: After-contractions of paced LAA exposed to ATX-II in the presence of ryanodine.

FIG. 11: After-contractions of paced LAA exposed to ATX-II in the presence of verapamil (Vrp).

FIG. 12: The effect of increasing concentrations of LOE-908 (12A) and SKF-96365 (12B) on the rate of after-contractions.

FIG. 13: The effect of increasing contractions of LOE-908 and SKF-96365 on the rate of abnormal automatic spontaneous contractions.

FIG. 14: The effects of LOE-908 and SKF-96365 on LAA experiencing abnormal automaticity.

FIG. 15: The effects of antibodies against Stim1 (which inhibit the ARC channel) on after-contractions.

FIG. 16: The effects of methyl-β-cyclodextrin (an ARC channel inhibitor) on after-contractions.

FIG. 17: The conventional model of the events leading to myocyte contraction. The numbered steps are (1) the depolarization impulse, (2) opening of sodium channels (I_{Na}), (3) opening of K and Ca channels (I_{Ca}) and repolarizing myocytes, (4) entry of voltage-dependent Ca into the myocyte and binding of voltage-dependent Ca to RyR, (5) calcium-induced release of calcium, (6) binding of Ca to myofilaments to activate contraction, (7) reuptake of Ca into the sarcoplasmic reticulum, resulting in relaxation of the myocyte.

FIG. 18: Known sequences of the Orai polypeptides comprising the ARC channel and conserved domains thereof.

FIG. 19: Known sequences of the CPLA2 polypeptide and conserved domains thereof.

FIG. 20: Known sequences of the STIM1 polypeptide and conserved domains thereof.

FIG. 21: Known aligned sequences of CPLA2 for nine model animal species.

FIG. 22: Known regions, modifications, variations, and point mutation data for CPLA2 as shown in the Uniprot KB database.

FIG. 23: Known regions, residue modifications, natural variants, and experimental results of modifying the native amino acid sequence of ORAI1 in Homo sapiens (this information is from the UniProtKB database)

FIG. 24: Known aligned sequences of Orai3 for five model animal species.

FIG. 25: Known aligned sequences of Orai1 for ten model animal species.

### DETAILED DESCRIPTION

### A. Definitions

The terms “prevention”, “prevent”, “preventing”, “suppression”, “suppress” and “suppressing” as used herein refer to a course of action (such as administering a compound or pharmaceutical composition of the present disclosure) initiated prior to the onset of a clinical manifestation of a disease state or condition so as to prevent or reduce such clinical manifestation of the disease state or condition. Such preventing and suppressing need not be absolute to be useful.

The terms “treatment”, “treat” and “treating” as used herein refers to a course of action (such as administering a compound or pharmaceutical composition) initiated after the onset of a clinical manifestation of a disease state or condition so as to eliminate or reduce such clinical manifestation of the disease state or condition. Such treating need not be absolute to be useful.

The term “treatment and prevention” as used herein refers to at least one of the acts of treatment or prevention. As such, it may be read to apply to treatment in the absence of prevention, prevention in absence of treatment, or concurrent treatment and prevention.

The term “in need of treatment” as used herein refers to a judgment made by a caregiver that a patient requires or will benefit from treatment. This judgment is made based on a variety of factors that are in the realm of a caregiver's expertise, but that includes the knowledge that the patient is ill, or will be ill, as the result of a condition that is treatable by a method, compound or pharmaceutical composition of the disclosure.

The term “in need of prevention” as used herein refers to a judgment made by a caregiver that a patient requires or will benefit from prevention. This judgment is made based on a variety of factors that are in the realm of a caregiver's expertise, but that includes the knowledge that the patient will be ill or may become ill, as the result of a condition that is preventable by a method, compound or pharmaceutical composition of the disclosure.

The term “individual”, “subject” or “patient” as used herein refers to any animal, including mammals, such as mice, rats, other rodents, rabbits, dogs, cats, swine, cattle, sheep, horses, or primates, and humans. The term may specify male or female or both, or exclude male or female.

The term “therapeutically effective amount” as used herein refers to an amount of a compound, either alone or as a part of a pharmaceutical composition, that is capable of having any detectable, positive effect on any symptom, aspect, or characteristics of a disease state or condition. Such effect need not be absolute to be beneficial.

The term “prodrug” as used herein includes functional derivatives of a disclosed compound which are readily convertible in vivo into the required compound. Thus, in the methods of treatment of the present disclosure, the term “administering” shall encompass the treatment of the various disease states/conditions described with the compound specifically disclosed or with a prodrug which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

The term “pharmaceutically acceptable salts” as used herein includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The term “polypeptide derivative” as defined herein refers to a polypeptide that includes a one or more fragments, insertions, deletions or substitutions. The polypeptide derivative may have an activity that is comparable to or increased (in one embodiment, 50% or more) as compared to the wild-type polypeptide activity and as such may be used to increase a polypeptide activity; alternatively, the polypeptide derivative may have an activity that is decreased (in one embodiment, less than 50%) as compared to the wild-type polypeptide activity and as such may be used to decrease a polypeptide activity. Derivatives that retain some activity of the native polypeptide are referred to herein as “functional variants.” In some cases the derivative will retain antigenic specificity of the polypeptide; such derivatives are referred to herein as “immunologically cross-reactive variants.”

A fragment of a given polypeptide is any polypeptide consisting of any number of adjacent amino acid residues having the same identity and order as any segment of the given polypeptide. Conservative modifications to the amino acid sequence of any fragment are also included (conservative substitutions are discussed below). Such fragments can be produced for example by digestion of the given polypeptide with an endoprotease (which will produce two or more fragments) or an exoprotease. A fragment may be of any length up to the length of the polypeptide. A fragment may be, for example, at least 3 residues in length. A fragment that is at least 6 residues in length will generally function as an antigenic group. Such groups would be expected by those of ordinary skill in the art to be cross-recognized by some antibodies specific for polypeptide. Fragments that are homologous to parts of the functional region of the polypeptide may have functional activity.

Derivatives will have some degree of homology with native polypeptide. For example, those skilled in the art would expect that most derivatives having from 95-100% homology with native polypeptide would retain the function of native polypeptide. It is also within the abilities of those skilled in the art to predict the likelihood that functionality would be retained by a homolog to a polypeptide within any one of the following ranges of homology: 75-100%, 80-100%, 85-100%, and 90-100%. Persons having ordinary skill in the art will understand that the minimum desirable homology can be determined in some cases by identifying a known non-functional homolog to the polypeptide, and establishing that the minimum desirable homology must be above the homology between the polypeptide and the known non-functional homolog. Persons having ordinary skill in the art will also understand that the minimum desirable homology can be determined in some cases by identifying a known functional homolog to the polypeptide, and establishing that the range of desirable homology may be as low as the homology between the native polypeptide and the known functional homolog.

The deletions, additions and substitutions can be selected, as would be known to one of ordinary skill in the art, to generate a desired polypeptide derivative. For example, it is not expected that deletions, additions and substitutions in a non-functional region of a polypeptide would alter activity. Likewise, conservative substitutions or substitutions of amino acids with similar properties are expected to be tolerated in a conserved region, and activity may be conserved. Of course non-conservative substitutions in these regions would be expected to decrease or eliminate activity. In addition, specific deletions, insertions and substitutions may impact, positively or negatively, a certain polypeptide activity but not impact another polypeptide activity.

Conservative modifications to the amino acid sequence of a polypeptide, and the corresponding modifications to the encoding nucleotides, will produce polypeptide derivatives having functional and chemical characteristics similar to those of the naturally occurring polypeptide. In contrast, substantial modifications in the functional and/or chemical characteristics of the polypeptide may be accomplished by selecting substitutions in the amino acid sequence of a polypeptide that differ significantly in their effect on maintaining (a) the structure of the molecular backbone in the area of the substitution, (b) the charge or hydrophobicity of the molecule at the binding site for a binding target, or (c) the bulk of a side chain.

For example, a “conservative amino acid substitution” may involve a substitution of a native amino acid residue with a nonnative residue such that there is little or no effect on the polarity or charge of the amino acid residue at that position. Furthermore, any native residue in the polypeptide may also be substituted with alanine.

Conservative amino acid substitutions also encompass non-naturally occurring amino acid residues which are typically incorporated by chemical peptide synthesis rather than by synthesis in biological systems. These include peptidomimetics and other reversed or inverted forms of amino acid moieties. It will be appreciated by those of skill in the art that nucleic acid and polypeptide molecules described herein may be chemically synthesized as well as produced by recombinant means.

Naturally occurring residues may be divided into classes based on common side chain properties: 1) hydrophobic: norleucine, Met, Ala, Val, Leu, Ile; 2) neutral hydrophilic: Cys, Ser, Thr, Asn, Gln; 3) acidic: Asp, Glu; 4) basic: His, Lys, Arg; 5) residues that influence chain orientation: Gly, Pro; and 6) aromatic: Trp, Tyr, Phe.

For example, non-conservative substitutions may involve the exchange of a member of one of these classes for a member from another class.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (Kyte et al., J. Mol. Biol., 157:105-131, 1982). It is known that certain amino acids may be substituted for other amino acids having a similar hydropathic index or score and still retain a similar biological activity.

In making changes based upon the hydropathic index, the substitution of amino acids whose hydropathic indices are within +/−2 may be used; in an alternate embodiment, the hydropathic indices are within +/−1; in yet another alternate embodiment, the hydropathic indices are within +/−0.5.

It is also understood in the art that the substitution of like amino acids can be made effectively on the basis of hydrophilicity. The greatest local average hydrophilicity of a polypeptide as governed by the hydrophilicity of its adjacent amino acids, correlates with a biological property of the protein.

The following hydrophilicity values have been assigned to amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids with hydrophilicity values within +/−2 may be used; in an alternate embodiment, the hydrophilicity values are within +/−1; in yet another alternate embodiment, the hydrophilicity values are within +/−0.5.

Desired amino acid substitutions (whether conservative or non-conservative) can be determined by those skilled in the art at the time such substitutions are desired. For example, amino acid substitutions can be used to identify important residues of the polypeptide, or to increase or decrease the affinity of the polypeptide with a particular binding target in order to increase or decrease a polypeptide activity.

Exemplary amino acid substitutions are set forth in Table I.

TABLE 1

| Original Amino | | Preferred |
|---|---|---|
| Acid | Exemplary substitution | substitution |
| Ala | Val, Leu, Ile | Val |
| Arg | Lys, Gln, Asn | Lys |
| Asn | Glu | Glu |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Ile, Val, Met, Ala, Phe, Norleucine | Ile |
| Lys | Arg, 1,4-diaminobutyric acid, Gln, Asn | Arg |
| Met | Leu, Phe, Ile | Leu |
| Phe | Leu, Val, Ile, Ala, Tyr | Leu |
| Pro | Ala, Gly | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

A skilled artisan will be able to determine suitable variants of a polypeptide using well known techniques. For identifying suitable areas of the molecule that may be changed without destroying activity, one skilled in the art may target areas not believed to be important for activity. For example, when similar polypeptides with similar activities from the same species or from other species are known, one skilled in the art may compare the amino acid sequence of a polypeptide to such similar polypeptides. With such a comparison, one can identify residues and portions of the molecules that are conserved among similar polypeptides. It will be appreciated that changes in areas of a polypeptide that are not conserved relative to such similar polypeptides would be less likely to adversely affect the biological activity and/or structure of the polypeptide. One skilled in the art would also know that, even in relatively conserved regions, one may substitute chemically similar amino acids for the naturally occurring residues while retaining activity (conservative amino acid residue substitutions). Therefore, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a polypeptide that correspond to amino acid residues that are important for activity or structure in similar polypeptides. One skilled in the art may opt for chemically similar amino acid substitutions for such predicted important amino acid residues of polypeptide.

One skilled in the art can also analyze the three-dimensional structure and amino acid sequence in relation to that structure in similar polypeptides. In view of that information, one skilled in the art may predict the alignment of amino acid residues of a polypeptide with respect to its three dimensional structure. One skilled in the art may choose not to make radical changes to amino acid residues predicted to be on the surface of the protein, since such residues may be involved in important interactions with other molecules. Moreover, one skilled in the art may generate test polypeptide derivatives containing a single amino acid substitution at each desired amino acid residue. The derivatives can then be screened using activity assays known to those skilled in the art and as disclosed herein. Such derivatives could be used to gather information about suitable substitution. For example, if one discovered that a change to a particular amino acid residue resulted in destroyed, undesirably reduced, or unsuitable activity, derivatives with such a change would be avoided. In other words, based on information gathered from such routine experiments, one skilled in the art can readily determine the amino acids where further substitutions should be avoided either alone or in combination with other mutations.

Numerous scientific publications have been devoted to the prediction of secondary structure from analyses of amino acid sequences (see Chou et al., Biochemistry, 13(2):222-245, 1974; Chou et al., Biochemistry, 113(2):211-222, 1974; Chou et al., Adv. Enzymol. Relat. Areas Mol. Biol., 47:45-148, 1978; Chou et al., Ann. Rev. Biochem., 47:251-276, 1979; and Chou et al., Biophys. J., 26:367-384, 1979). Moreover, computer programs are currently available to assist with predicting secondary structure of polypeptides. Examples include those programs based upon the Jameson-Wolf analysis (Jameson et al., Comput. Appl. Biosci., 4(1):181-186, 1998; and Wolf et al., Comput. Appl. Biosci., 4(1):187-191; 1988), the program PepPlot™ (Brutlag et al., CABS, 6:237-245, 1990; and Weinberger et al., Science, 228:740-742, 1985), and other new programs for protein tertiary structure prediction (Fetrow. et al., Biotechnology, 11:479-483, 1993).

Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural data base (PDB) has provided enhanced predictability of secondary structure, including the potential number of folds within a polypeptide's or protein's structure (see Holm et al., Nucl. Acid. Res., 27(1):244-247, 1999).

Additional methods of predicting secondary structure include “threading” (Jones, D., Curr. Opin. Struct. Biol., 7(3):377-87, 1997; Suppl et al., Structure, 4(1):15-9, 1996), “profile analysis” (Bowie et al., Science, 253:164-170, 1991; Gribskov et al., Meth. Enzym., 183:146-159, 1990; and Gribskov et al., Proc. Nat. Acad. Sci., 84(13): 4355-4358, 1987), and, “evolutionary linkage” (See Home, supra, and Brenner, supra).

B. Preventing and/or Reversing Arrhythmia

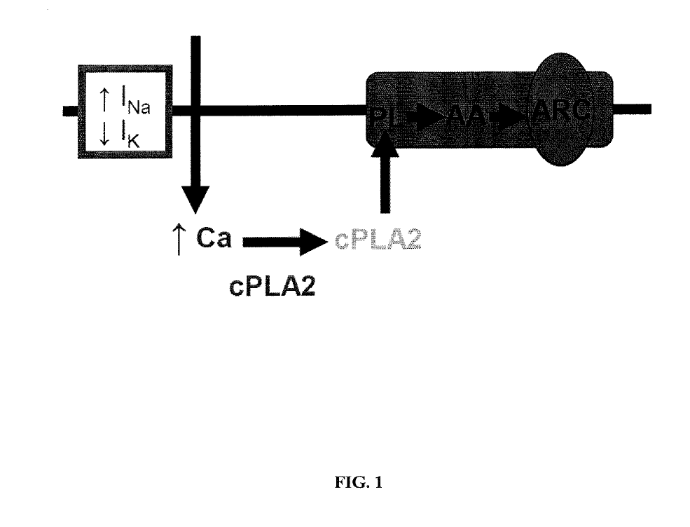

It has been unexpectedly discovered that the ARC channel plays a key role in after-depolarization events that lead to arrhythmia. Without wishing to be bound by any single hypothetical model, the apparent role played by the ARC channel in after-depolarization events is illustrated in FIGS. 1-3. As shown in FIG. 1, an increase in the late sodium current (I_{Na}) or a decrease in the potassium current (I_{K}) causes an increase in intracellular calcium concentrations (↑Ca). Proteins that depend on the concentration of calcium for activity, such as cytosolic phospholipase A2 (CPLA2), are then activated. Once active, CPLA2 binds to the membrane and catalyzes the hydrolysis of phospholipids to release arachidonic acid. Arachidonic acid activates the ARC channel.

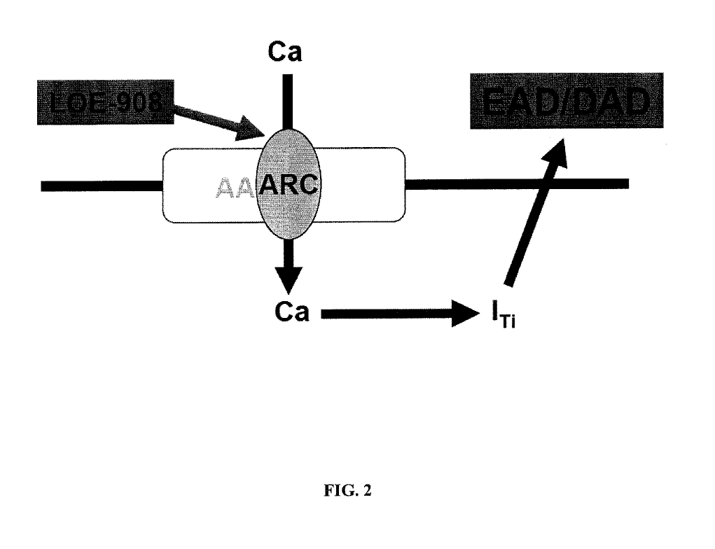

As shown in FIG. 2, activation of the ARC channel imports additional extracellular calcium into the cell, leading to the production of the depolarizing transient inward current (I_{Ti}). The depolarizing transient inward current activates ectopic myocyte depolarization that initiates calcium-induced calcium release from the sarcoplasmic reticulum, resulting in an after-depolarization event (EAD/DAD) and the resulting after-contraction. An inhibitor of the ARC channel, such as LOE-908 as shown, will prevent the ARC channel-mediated entry of calcium, blocking the cascade that results in after-depolarization and arrhythmia.

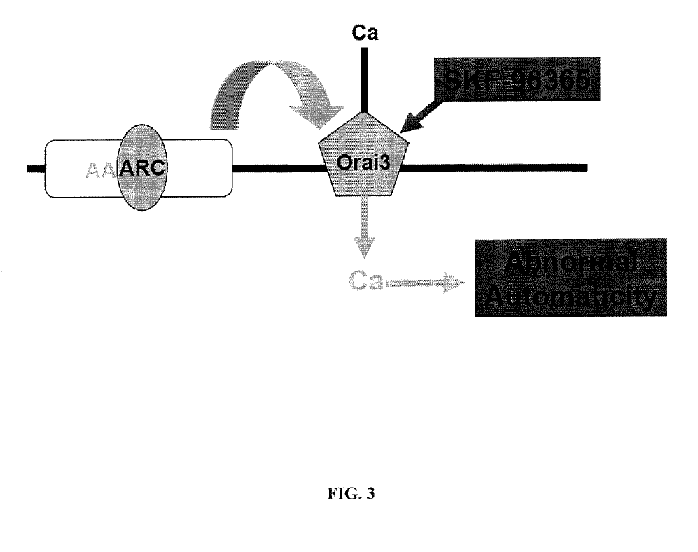

As shown in FIG. 3, the activation of ARC channels results in SOCC-linked abnormal automaticity. This is defined as the production of spontaneous electrical depolarizations in heart muscle as a result of calcium entry through the SOCC and signaling events that arise from this calcium entry. Panel (i) of FIG. 3A shows conditions in which the ARC and SOCC are inactive and the internal calcium store is full. Panel (ii) illustrates a model wherein low-level ARC activation causes partial depletion of cell calcium stores and low-level activation of the SOCC. Both appear to be necessary to provoke after-depolarization events (such as EAD) as well as attendant after-contractions. In this setting ARC inhibitors (such as LOE-908) and SOCC inhibitors (such as SKF-96365) will block after-depolarization events.

Panel (iii) show that prolonged or intense ARC signaling will deplete cell calcium stores substantially or completely. This significantly activates SOCC-linked calcium entry. It now appears that SOCC-mediated calcium entry causes abnormal automaticity, an arrhythmic activity that occurs independently of triggered activity. In this setting, ARC channel inhibitors like LOE-908 will not block SOCC-linked automaticity, but SOCC inhibitors like SKF-96365 will.

FIG. 3B shows the conventional model of the reciprocal relationship between ARC channel activity and SOCC activation. As can be seen on the left side of the top panel and in the bottom left panel, repeated activation of the ARC channel results in repeated calcium entry and release from cytosolic stores (ER). As can be seen in on the right side of the upper panel and in the lower right panel, upon ER store depletion, the SOCC opens to replenish internal stores.

In many arrhythmias, after-depolarization events initiate triggered activity, which is closely followed by rapid automatic activity. It has been unexpectedly discovered that the process of ARC channel activation initiates after-depolarization events. It has also been unexpectedly discovered that SOCC calcium entry can produce abnormal automaticity. These finding were not expected based on conventional models describing ARC-SOCC interaction in non-excitable cells, because such models never contemplated a link between ARC channel activation or SOCC channel activation to arrhythmic events. Thus, inhibitors of SOCC activity (such as SKF-96365 as shown) can interrupt the cascade that results in after-depolarization and arrhythmia that is initiated by ARC channel calcium flux. Such inhibitors of SOCC activity are therefore also inhibitors of ARC channel-related arrhythmia.

Methods are provided for reversing arrhythmia in a cardiac muscle, as are methods for preventing arrhythmia in a cardiac muscle. Also provided are uses of the active compounds disclosed herein (below) for reversing arrhythmia in a cardiac muscle, as are uses of the active compounds disclosed herein for preventing arrhythmia in a cardiac muscle. Also provided are uses of the active compounds disclosed herein for producing a pharmaceutical composition for reversing arrhythmia in a cardiac muscle, as are uses of the active compounds disclosed herein for producing a pharmaceutical composition for preventing arrhythmia in a cardiac muscle.

The methods and uses provided comprise contacting the cardiac muscle with any of the active compounds described herein at a concentration sufficient to treat or prevent the arrhythmia. Such concentrations can be determined by those of ordinary skill in the art, for example by use of the cardiac muscle assay described below. For example, the active compound may be present in a concentration of about 10 μM. As described in the examples below, the compound LOE-908 effectively reduces ectopic contractions in cardiac muscle at concentrations as low as 1 μM, and at 20 μM no ectopic contractions are observed. Accordingly, the concentration of LOE-908 in the current methods and uses may be at least 1 μM, at least 5 μM, at least 10 μM, at least 20 μM, at least 40 μM, any range interval therein, and about any of the foregoing concentrations. As described in the examples below, antibodies to Stim1 effectively eliminate ectopic contractions at concentrations as low as 3 μg/mL. Accordingly, the concentration of antibodies to Stim1 may be 3 μg/mL, at least 3 μg/mL, or about any of the foregoing concentrations. As described in the examples below, methyl-β-cyclodextrin effectively eliminates ectopic contractions at concentrations as low as 10 mM. Accordingly, the concentration of methyl-β-cyclodextrin may be 10 mM, at least 10 mM, or about any of the foregoing concentrations. As described in the examples below, aristolochic acid and amylcinnamoyl-anthranilic acid (ACA) suppress after contractions; ACA suppresses after contractions at concentrations as low as 10 μM. The examples below demonstrate that ACA has an observed IC50 of 10±4.5 μM and aristolochic acid has an observed IC50 of 31±5 μM. Accordingly, the concentration of ACA in the current methods and uses may be equal to or greater than 5.5 μM, 10 μM, 14.5 μM, 30 μM, 50 μM, or about any of the foregoing ranges or concentrations.

The arrhythmia may be of any type that is caused by an after-depolarization, such as a triggered arrhythmia. The after-depolarization may be an early after-depolarization or a delayed after-depolarization. Such methods and uses comprise suppressing the after-depolarization.

The disclosure further provides methods of treating cardiac arrhythmia in a subject, methods of preventing cardiac arrhythmia in a subject, uses of any of the pharmaceuticals disclosed herein for treating cardiac arrhythmia in a subject, and uses of any of the pharmaceuticals disclosed herein for preventing cardiac arrhythmia in a subject. The methods and uses comprise administering to the subject any of the pharmaceutical compositions disclosed herein in a therapeutically effective amount. In a specific embodiment, the pharmaceutical composition comprises an inhibitor of CPLA2.

The subject may be a subject in need of treatment or prevention of arrhythmia. Whether a subject is in such need can be determined by a medical professional. For example, the presence of extant arrhythmia is often diagnosed by an electrocardiogram, echocardiogram, a Holter monitor, a stress test, an event recorder (loop recorder), magnetic source imaging, a tilt table test, or the electrophysiology (EP) study. Symptoms indicative of the presence of arrhythmia include lightheadedness or dizziness, palpitations, fatigue, chest pain, shortness of breath, and fainting. Frequent occurrence of such symptoms could indicate the subject to be in need of prevention of arrhythmia; during such symptoms the subject could be deemed to be in need of treatment of arrhythmia. The subject may be deemed in need of treatment or prevention of arrhythmia by virtue of being a member of a high-risk group. Known risk factors for arrhythmia include previous heart attack, previous cardiomyopathy, enlarged heart, abnormal heart valves, congenital heart defects, hypertension, infection of the heart or the pericardial membrane, diabetes, sleep apnea, hyperthyroidism, and hypothyroidism.

The methods and uses may be accompanied by complimentary treatments or preventions, which may in some cases provide synergistic effects.

### C. Active Compounds

The present disclosure provides for compounds that inhibit ARC channel activity, either directly or through inhibition of expression, either in vitro or in vivo. The present disclosure also provides compounds that indirectly inhibit ARC channel activity by either stimulating the activity of a molecule that inhibits ARC channel activity or by inhibiting the activity of a molecule that stimulates ARC channel activity (the ARC channel and those molecules that stimulate the ARC channel are referred to here as “target molecules”). Such inhibitors are referred to herein as “active compounds.”

The active compound may also be a compound that inhibits SOCC activity, either directly or through inhibition of expression, either in vitro or in vivo. The present disclosure also provides compounds that indirectly inhibit SOCC activity by either stimulating the activity of a molecule that inhibits SOCC activity or by inhibiting the activity of a molecule that stimulates SOCC activity. The SOCC and those molecules that stimulate the SOCC may also be considered target molecules.

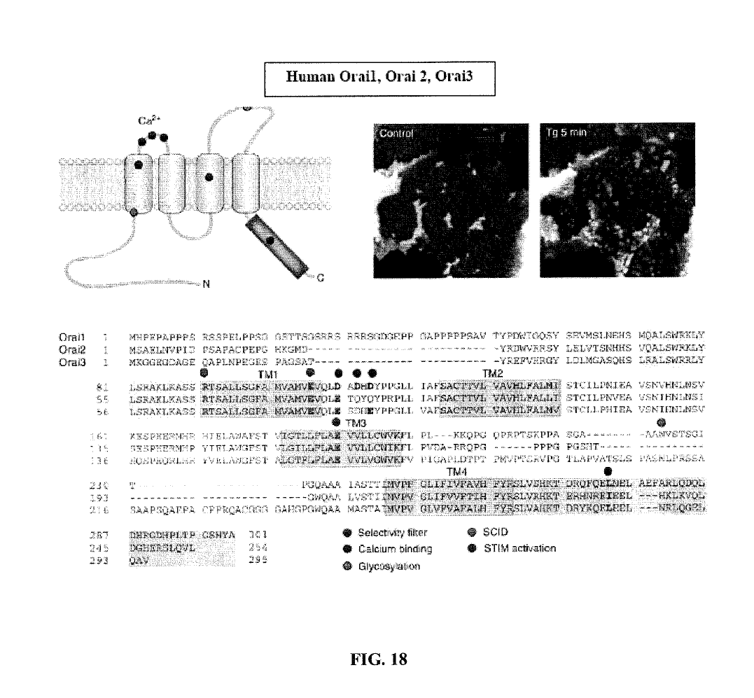

The ARC channel, any of its constituent polypeptides, and conservative variants thereof may be target molecules. The ARC channel is a pentamer containing three copies of ORAI1 and two copies of the ORAI3 protein (Mignen et al. J Physiology (2009) 581:4818-4197). In contrast, the SOCC is a tetramer containing four copies of the ORAI1 protein (Mignen et al. J Physiology (2008) 586:419-425). FIG. 18 shows the functional domains and the amino acid primary structure of human ORAI1, ORAI2 (which is not a known component of the ARC channel), and ORAI3. In this diagram, TM stands for the ‘transmembrane’ portion of the ORAI polypeptide which traverses cell membranes and forms the ion channel. In the Uniprot KB/Swiss-Prot data base human ORAI1 is designated by the accession number Q96D31 and ORAI3 is designated by Q9BRQ5.

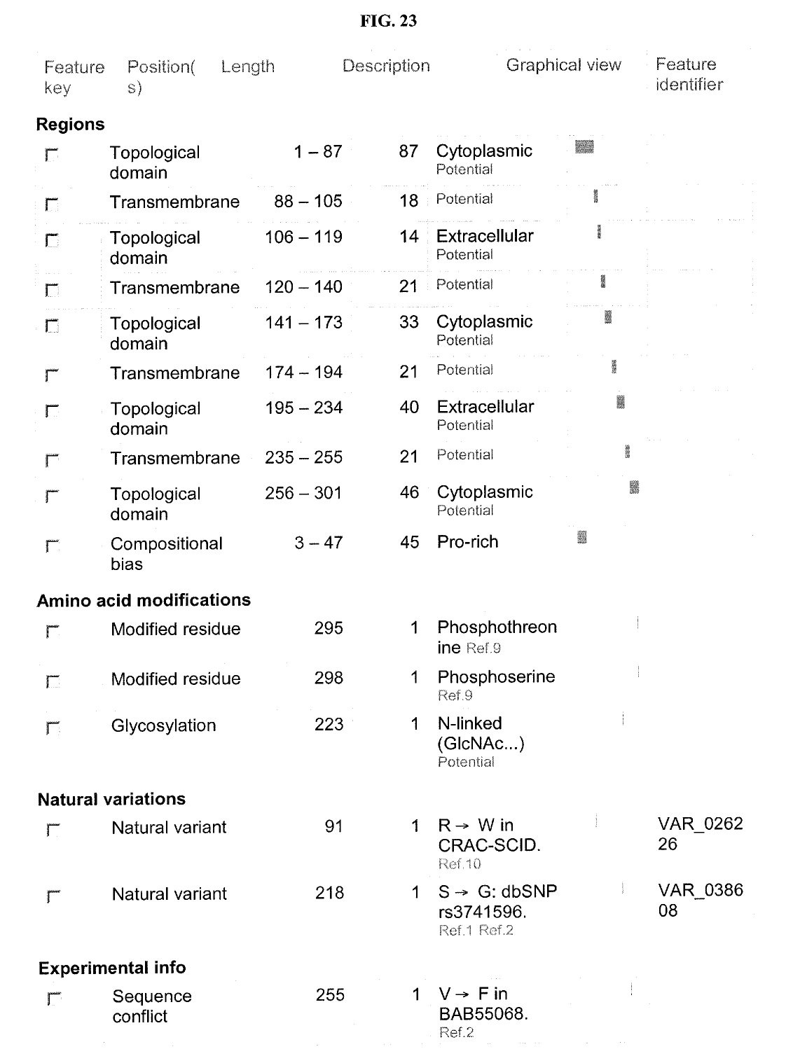

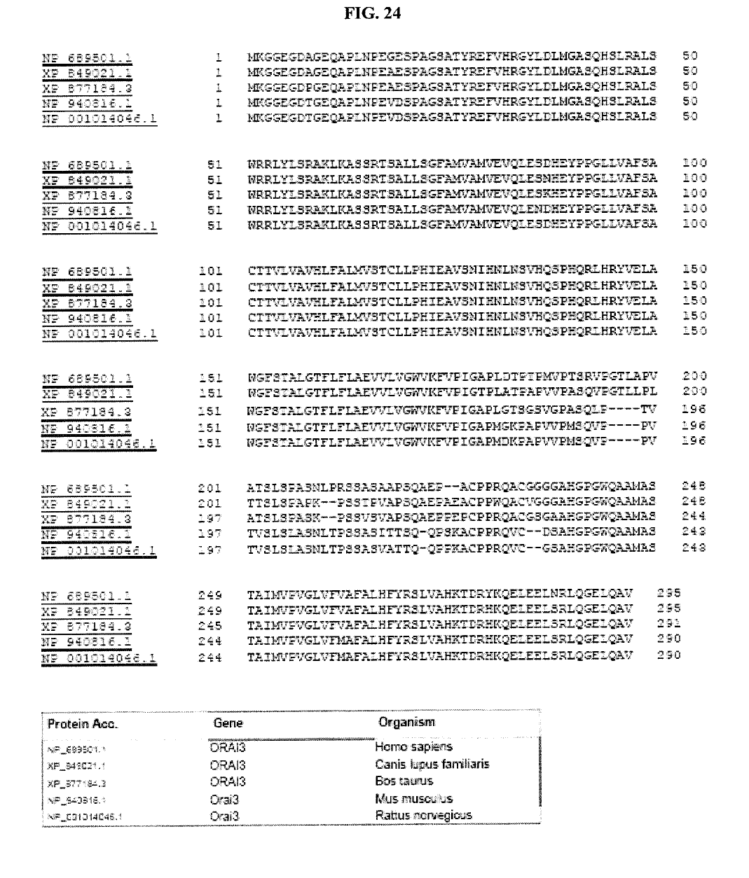

FIG. 23 summarizes regions, residue modifications, natural variants, and experimental results of modifying the native amino acid sequence of ORAI1 in Homo sapiens (this information is from the UniProtKB database). FIG. 24 shows comparative primary sequences for the ORAI3 polypeptides in Homo sapiens, Canis lupus familiaris, Bos taurus, Rattus norvegicus, and Mus musculus. Regions of relatively high homology between various species (conserved regions) can be assumed to be of functional importance in the absence of confirmatory data.

The target molecule may be a phospholipase, an inhibitor of cellular voltage-independent calcium homeostasis, or an inhibitor of a voltage-independent calcium channel. The voltage-independent calcium channel may be, for example, a channel regulated by Arachidonic aid or by intracellular calcium stores. The phospholipase may be, for example, a phospholipase that is activated by an increase in intracellular calcium concentration (such as CPLA2).

Arachidonic acid and derivatives of arachidonic acid that function to activate the ARC channel may be target molecules. Arachidonic acid is a fatty acid that is common in cells. It is a 20 carbon unsaturated fatty acid (20:4(ω-6)). Among other known pathways for arachidonic acid synthesis, arachidonic acid is produced by the hydrolysis of phospholipids catalyzed by cytosolic phospholipase A2.

Cytosolic phospholipase A2 (CPLA2) and conservative variants thereof may be target molecules. CPLA2 is an 85 kDa protein that normally resides in the cytosol. Following an increase in intracellular cell calcium concentration, CPLA2 binds calcium and translocates to a cell membrane where it inserts itself due to the phospholipid binding activity of the C2-binding domain (the C2-binding domain being critical to membrane insertion and proper function of the protein). Membrane-bound CPLA2 then hydrolyzes membrane phospholipids to form arachidonic acid, the activating signal for ARC. CPLA2 thus increases ARC channel activity.

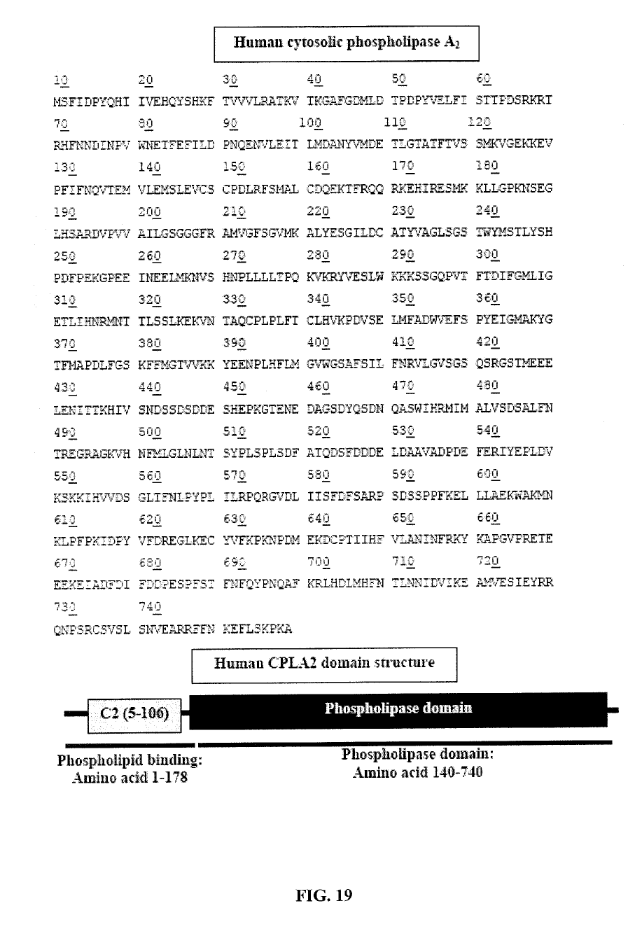

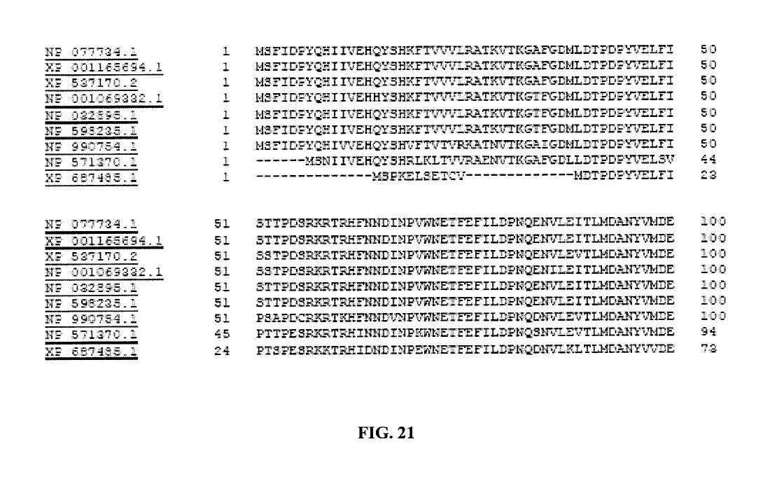

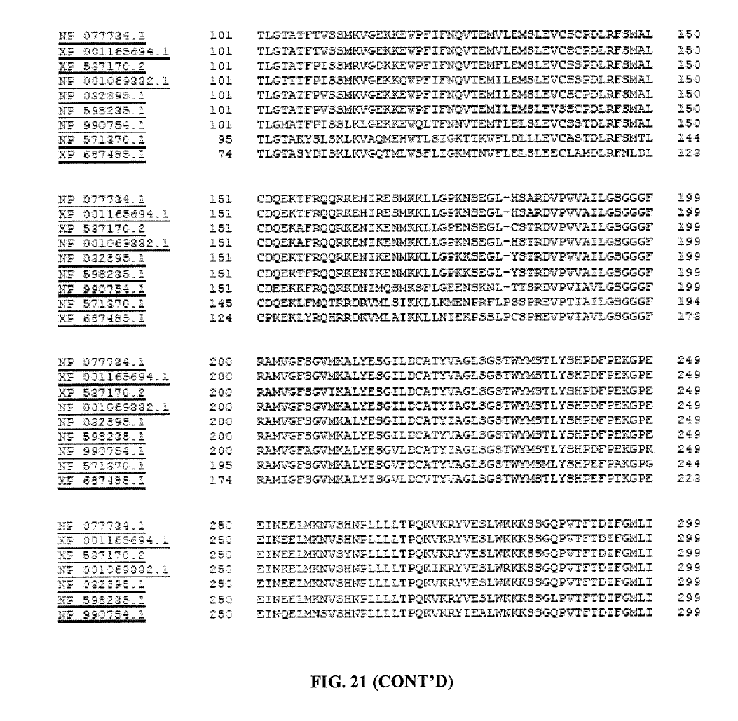

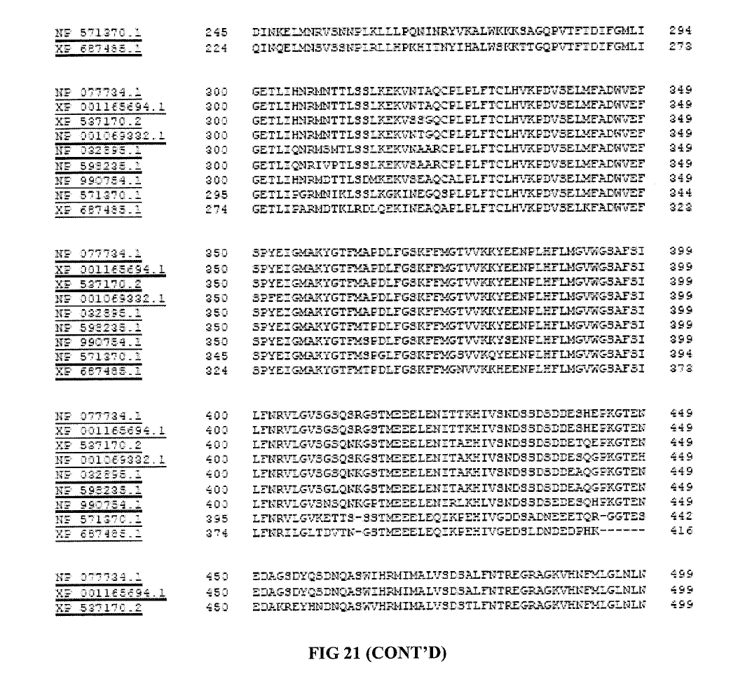

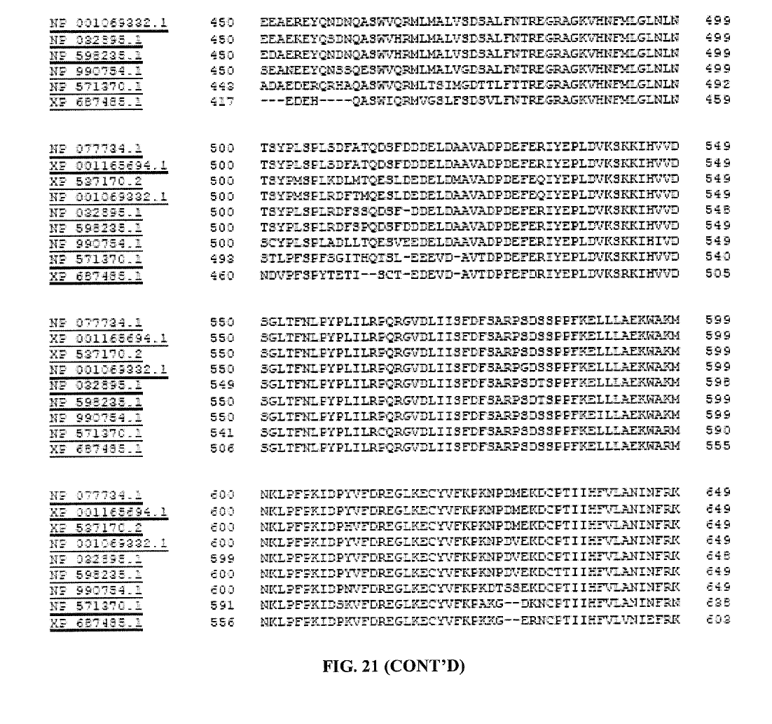

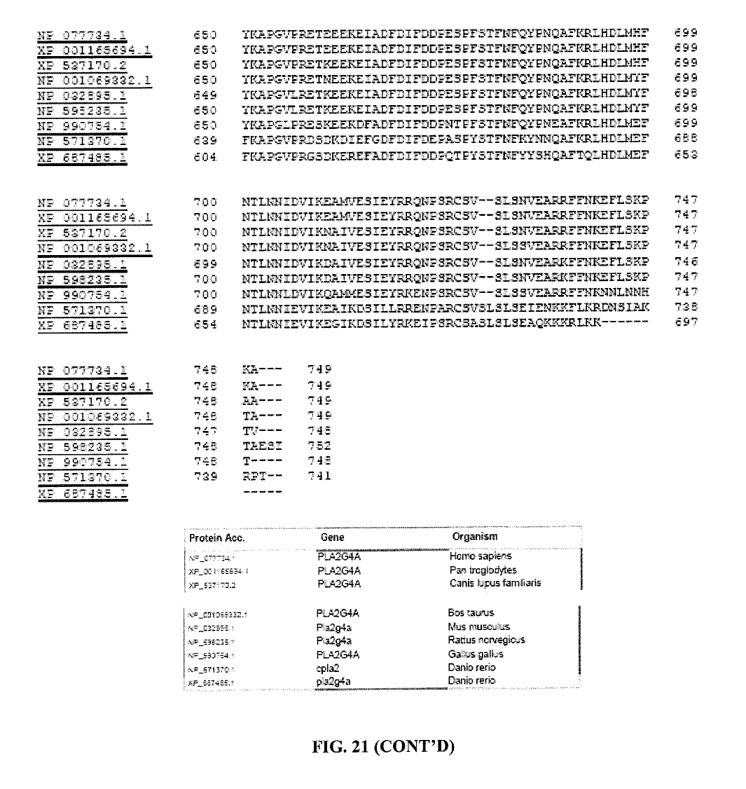

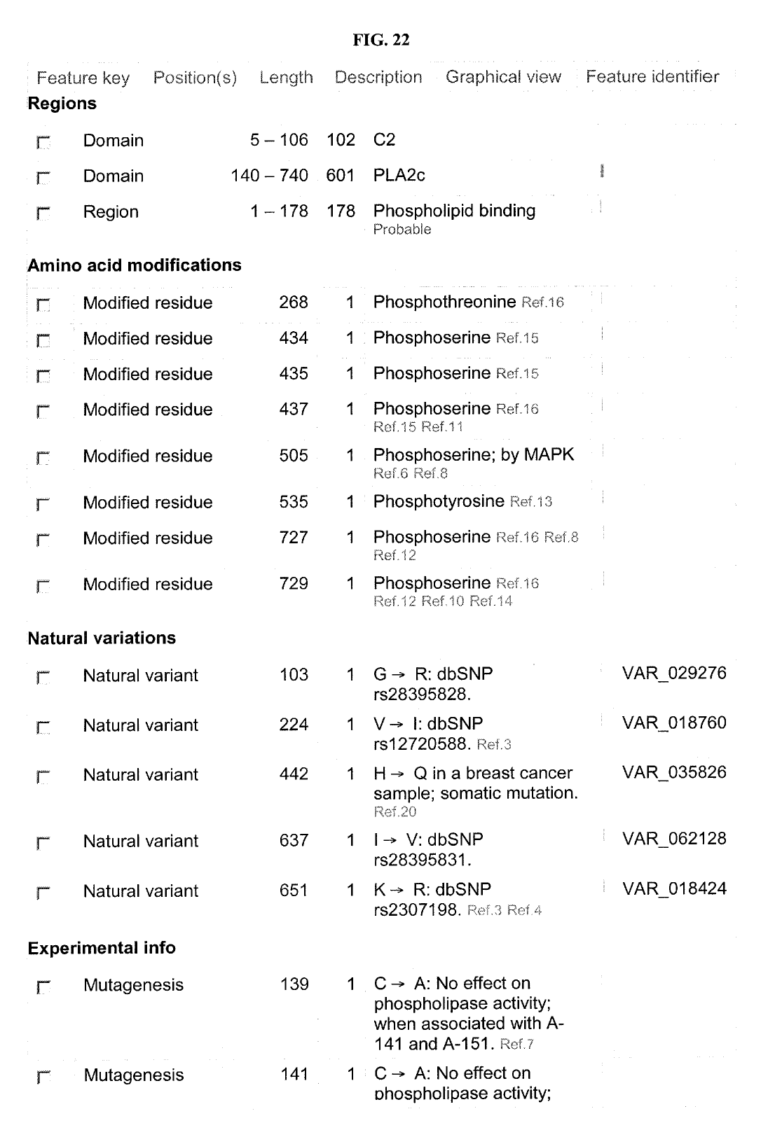

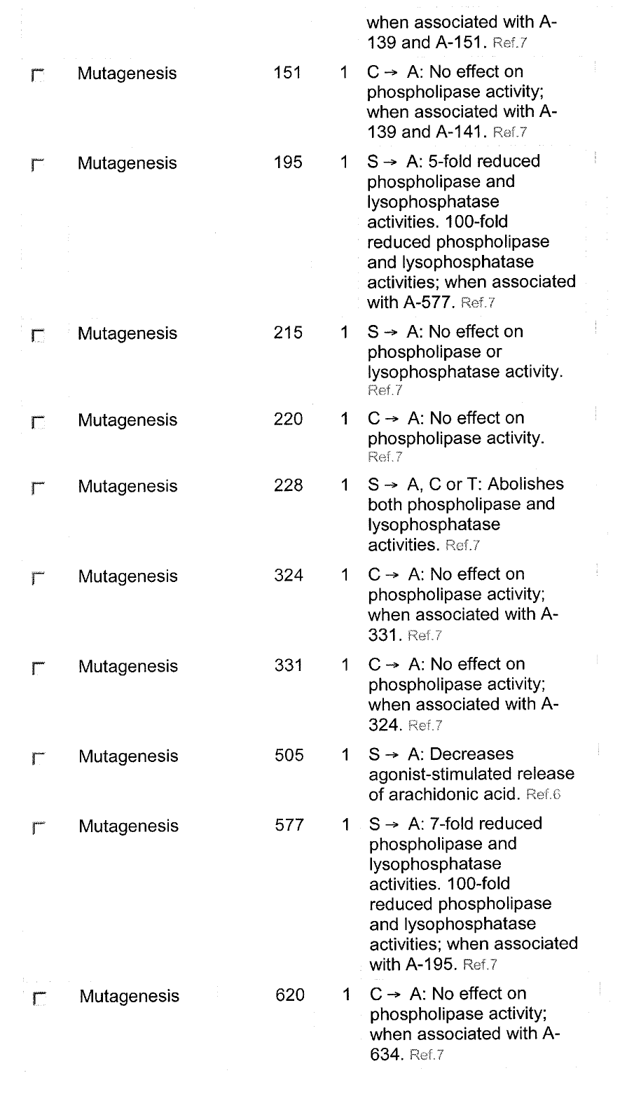

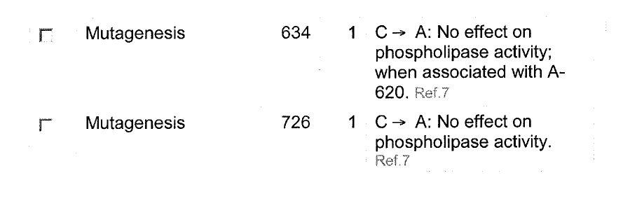

FIG. 19 shows the primary amino acid structure for human CPLA2 and specifies the C2 domain (which is responsible for phospholipid binding) and the phospholipase domain. CPLA2 is designated accession number P47712 in the UniProt KB/Swiss-Prot databases. FIG. 21 shows comparative primary sequences for the CPLA2 polypeptides in Homo sapiens, Pan troglodytes, Canis lupus familiaris, Bos taurus, Mus musculus, Rattus norvegicus, Gallus gallus, and Danio rerio. Regions of relatively high homology between various species (conserved regions) can be assumed to be of functional importance in the absence of confirmatory data. FIG. 22 summarizes regions, residue modifications, natural variants, and experimental results of modifying the native amino acid sequence of CPLA2 in Homo sapiens (this information is from the UniProtKB database).

As illustrated in FIG. 22, various domains, modified amino acids, natural variants, and experimentally produced variants of CPLA2 have been described. Absent contrary evidence, it would be assumed that non-conservative substitutions of a functional domain or region could compromise the function of the polypeptide. In addition, absent contrary evidence, it would be assumed that substitutions of modified amino acids with an amino acid that cannot be chemically modified in the same way could compromise the function of the polypeptide. In addition, absent contrary evidence, it would be assumed that natural variants that are not known to be associated with some dysfunction or disorder do not compromise the function of the polypeptide. Based on experimental evidence, certain mutations to human CPLA2 eliminate or reduce its function (see FIG. 22). Such mutations are not functional variants (conservative variants) of CPLA2. However, experimental evidence showing that a given point mutation does not affect the activity of CPLA2 indicates that a variant having that point mutation is a functional variant.

Stroma1 interaction molecule 1 is (STIM1) is a 32 kDa protein which acts as a calcium sensor for the SOCC and the ARC channels. Most STIM1 resides in intracellular membranes, where it activates SOCC in response to the depletion of intracellular calcium stores. It also resides in the cell plasma membrane, where it acts as an activator of ARC channels.

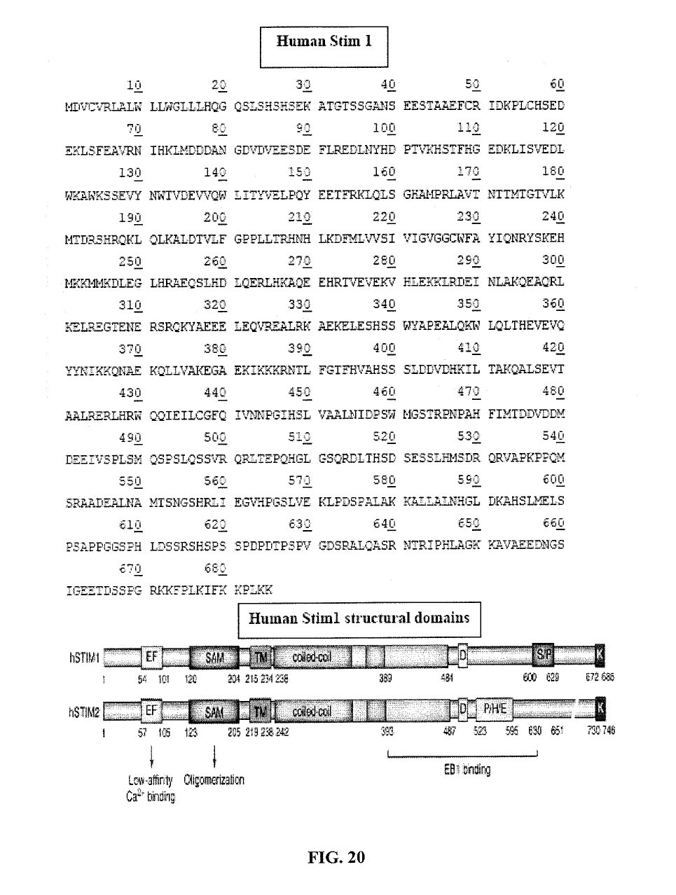

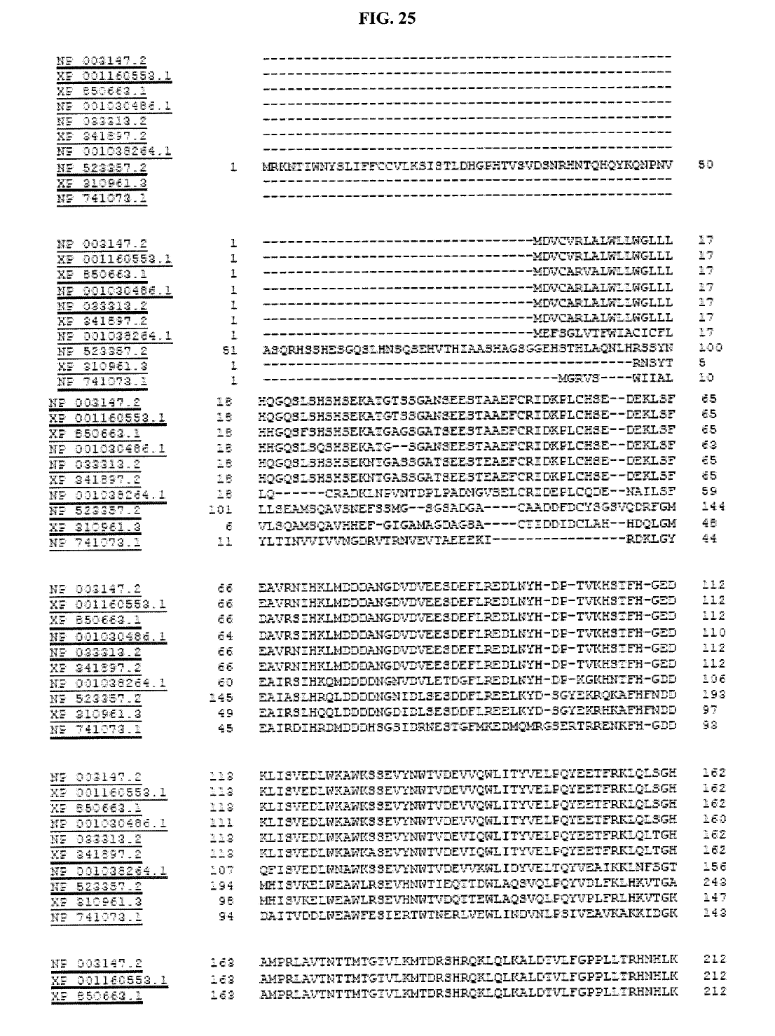

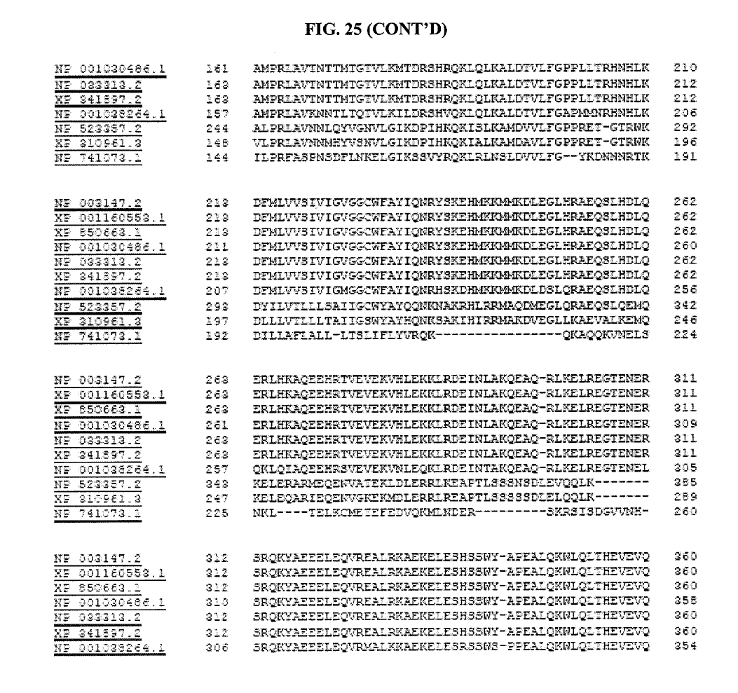

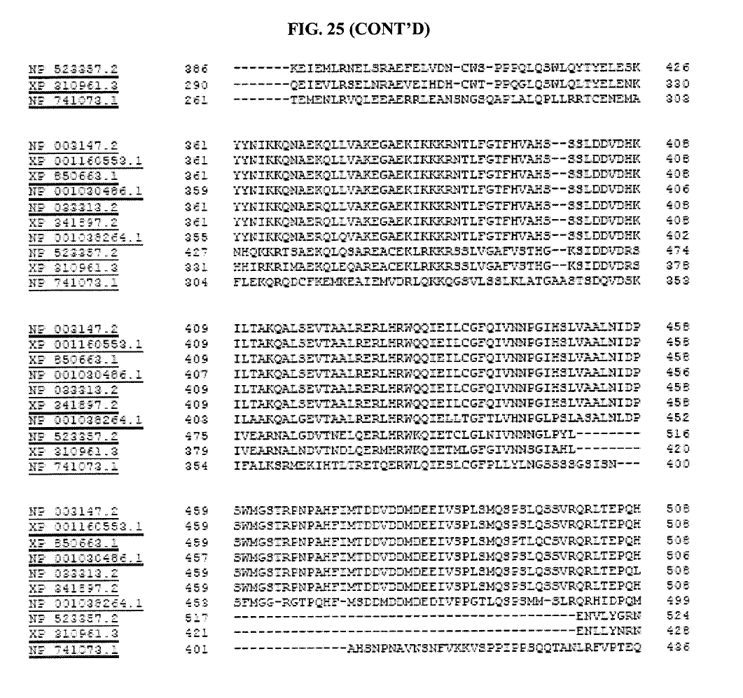

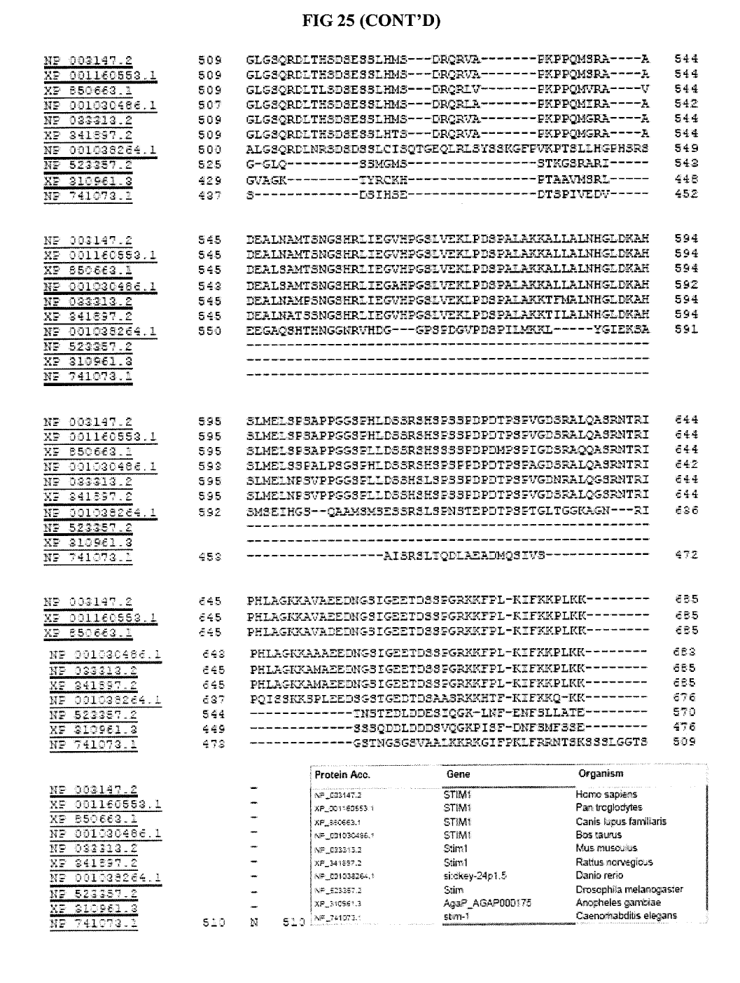

FIG. 20 shows the primary amino acid structure and established functional domains of human STIM1. In this figure “EF” is the “EF hand” domain which binds calcium and acts as a calcium sensor. “TM” is the transmembrane domain with which the protein is inserted into and through the intracellular or plasma membrane, and which remains in contact with the hydrophobic region of the membrane after insertion. The SAM domain is involved in STIM1 protein aggregation and SOCC activation. The coiled-coil domain imparts the 3-dimensional structure to STIM1. The UniProtKB/SwissProt data base designates human Stim1 the accession number Q13586. FIG. 25 shows comparative primary sequences for the STIM1 polypeptides in Homo sapiens, Pan troglodytes, Canis lupus familiaris, Bos taurus, Mus musculus, Rattus norvegicus, Danio rerio, Drosophila melanogaster, Anopheles gambiae, and Caenorhabditis elegans. Regions of relatively high homology between various species (conserved regions) can be assumed to be of functional importance in the absence of confirmatory data.

Active compounds can exert their effect on the ARC channel activity via changes in expression, post-translational modifications or by other means. For example, an active compound might inhibit ARC channel activity by inhibiting the transcription of a gene that encodes the target molecule. The active compound might also inhibit the translation of the RNA transcript of a gene for the target molecule to form its corresponding polypeptide. The active compound might also inhibit the activity of the translated polypeptide. Such inhibition of activity may occur by many mechanisms. Examples include prevention of proper folding of the polypeptide, prevention of the proper assembly of a polypeptide monomer with another monomer, competitive inhibition, and allosteric inhibition. Suitable compounds include, but are not limited to, polypeptides, functional nucleic acids, carbohydrates, antibodies, small molecules, or any other molecule which decrease the activity of the target molecule. Such compounds may be identified in the methods of screening discussed herein.

1. Nucleic Acid Inhibitors

In one embodiment, the active compounds of the present disclosure are functional nucleic acids. Functional nucleic acids are nucleic acid molecules that carry out a specific function in a cell, such as binding a target molecule or catalyzing a specific reaction.

Such functional nucleic acids may inhibit the activity of the ARC channel, the SOCC, or a molecule that increases the activity of either. Functional nucleic acids include but are not limited to antisense molecules, aptamers, ribozymes, triplex forming molecules, small interfering RNA (siRNA), RNA interference (RNAi), and external guide sequences (EGS). In one embodiment, a siRNA could be used to reduce or eliminate expression of the target molecule.

Antisense molecules are designed to interact with a target nucleic acid through either canonical or non-canonical base pairing. The interaction of the antisense molecule and the target nucleic acid is designed to promote the destruction of the target nucleic acid through, for example, RNAseH mediated RNA-DNA hybrid degradation. Alternatively, the antisense molecule is designed to interrupt a processing function that normally would take place on the target nucleic acid, such as transcription or replication. Antisense molecules can be designed based on the sequence of the target nucleic acid (such as a nucleic acid encoding a target molecule, such as an ARC channel polypeptide component, an SOCC polypeptide component, or a polypeptide that stimulates the activity or expression of the ARC molecule or the SOCC). In a specific embodiment the antisense molecule is complimentary to at least a portion of a CPLA2 gene. Numerous methods for optimization of antisense efficiency by finding the most accessible regions of the target nucleic acid molecule exist. Exemplary methods include, but are not limited to, in vitro selection experiments and DNA modification studies using DNA molecular screening (DMS) and DEPC.

Aptamers are molecules that interact with a target nucleic acid, preferably in a specific way. Typically aptamers are small nucleic acids ranging from 15-50 bases in length that fold into defined secondary and tertiary structures, such as stem-loops or G-quartets. Representative examples of how to make and use aptamers to bind a variety of different target molecules can be found in, for example, U.S. Pat. Nos. 5,476,766 and 6,051,698 (which are hereby incorporated by reference only for this teaching). The secondary structure inhibits expression of the polypeptide encoded by the gene or inhibits a processing function as discussed above. Aptamers of the present disclosure may interact with a nucleic acid encoding a target molecule, such as an ARC channel polypeptide component, an SOCC polypeptide component, or a polypeptide that stimulates the activity or expression of the ARC molecule or the SOCC. In a specific embodiment the aptamer interacts with CPLA2.

Ribozymes are nucleic acid molecules that are capable of catalyzing a chemical reaction, either intra-molecularly or inter-molecularly. There are a number of different types of ribozymes that catalyze nuclease or nucleic acid polymerase type reactions which are based on ribozymes found in natural systems, such as, but not limited to, hammerhead ribozymes, hairpin ribozymes and Tetrahymena ribozymes. There are also a number of ribozymes that are not found in natural systems, but which have been engineered to catalyze specific reactions de novo (including, but not limited to, those described in U.S. Pat. Nos. 5,807,718, and 5,910,408, which are hereby incorporated by reference only for this teaching). Ribozymes may cleave RNA or DNA substrates. Representative examples of how to make and use ribozymes to catalyze a variety of different reactions can be found in U.S. Pat. Nos. 5,837,855; 5,877,022; 5,972,704; 5,989,906; and 6,017,756 (which are hereby incorporated by reference only for this teaching). Ribozymes of the present disclosure may cleave a nucleic acid encoding a target molecule, such as an ARC channel polypeptide component, an SOCC polypeptide component, or a polypeptide that stimulates the activity or expression of the ARC molecule or the SOCC. In a specific embodiment the Ribozyme cleaves CPLA2.

Triplex forming functional nucleic acid molecules are nucleic acid molecules that can interact with either double-stranded or single-stranded nucleic acid. When triplex forming nucleic acids interact with a target region of another nucleic acid, a structure called a triplex is formed, in which three strands of DNA form a complex dependant on both Watson-Crick and Hoogsteen base-pairing. Triplex molecules can bind target regions of nucleic acids with high affinity and specificity. Representative examples of how to make and use triplex forming molecules to bind a variety of different target molecules can be found in U.S. Pat. Nos. 5,650,316; 5,683,874; 5,693,773; 5,834,185; 5,869,246; 5,874,566; and 5,962,426 (which are hereby incorporated by reference only for this teaching). Triplex molecules of the present disclosure may bind a nucleic acid encoding a target molecule, such as an ARC channel polypeptide component, an SOCC polypeptide component, or a polypeptide that stimulates the activity or expression of the ARC molecule or the SOCC. In a specific embodiment the triplex molecule binds to a nucleic acid sequence encoding CPLA2.

External guide sequences (“EGS”) are molecules that bind a target nucleic acid forming a complex, which is recognized by RNase P. RNaseP then cleaves the target nucleic acid molecule. EGS can be designed to specifically target an RNA molecule of choice. Representative examples of how to make and use EGS molecules to facilitate cleavage of a variety of different target molecules may be found in U.S. Pat. Nos. 5,168,053; 5,624,824; 5,683,873; 5,728,521; 5,869,248; and 5,877,162 (which are hereby incorporated by reference only for this teaching). External guide sequences of the present disclosure may bind a nucleic acid encoding a target molecule, such as an ARC channel polypeptide component, an SOCC polypeptide component, or a polypeptide that stimulates the activity or expression of the ARC molecule or the SOCC. In a specific embodiment the EGS binds a nucleic acid encoding CPLA2.

Gene expression can also be effectively silenced in a highly specific manner through RNA interference (“RNAi”). Small interfering RNA (“siRNA”) is a double-stranded RNA that can induce sequence-specific post-transcriptional gene silencing, thereby decreasing or even inhibiting gene expression from a target nucleic acid. In one example, an siRNA triggers the specific degradation of homologous RNA molecules, such as mRNAs, within the region of sequence identity between both the siRNA and the target RNA. Sequence specific gene silencing can be achieved in mammalian cells using synthetic, short double-stranded RNAs that mimic the siRNAs produced by the enzyme dicer. siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. siRNA can also be synthesized in vitro using kits such as Ambion's SILENCER® siRNA Construction Kit (Ambion, Austin, Tex.). Small interfering RNAs of the present disclosure may trigger the degradation of a nucleic acid encoding a target molecule, such as an ARC channel polypeptide component, an SOCC polypeptide component, or a polypeptide that stimulates the activity or expression of the ARC molecule or the SOCC. In a specific embodiment the siRNA triggers the degradation of a nucleic acid encoding CPLA2.

2. Antibody Inhibitors

Polypeptides that inhibit target molecule activity include antibodies with antagonistic or inhibitory properties. In addition to intact immunoglobulin molecules, fragments, chimeras, or polymers of immunoglobulin molecules are also useful in the methods taught herein, as long as they are chosen for their ability to inhibit target molecule activity. The antibodies can be tested for their desired activity using in vitro assays, or by analogous methods, after which their in vivo therapeutic or prophylactic activities are tested according to known clinical testing methods.

The term antibody is used herein in a broad sense and includes both polyclonal and monoclonal antibodies. Monoclonal antibodies can be made using any known procedure. For example, disclosed monoclonal antibodies can be prepared using hybridoma methods, such as those described by Kohler and Milstein, Nature, 256:495 (1975) (which is incorporated by reference herein only for this teaching). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro. The monoclonal antibodies may also be made by recombinant DNA methods, such as those described in U.S. Pat. No. 4,816,567 (which is hereby incorporated by reference only for this teaching). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, as described in U.S. Pat. No. 5,804,440 and U.S. Pat. No. 6,096,441 (which are hereby incorporated by reference only for this teaching).

Antibody fragments include Fv, Fab, Fab′ or other antigen binding portion of an antibody. Digestion of antibodies to produce fragments thereof can be accomplished using routine techniques known in the art. For instance, digestion can be performed using a protease, such as papain or pepsin. Examples of papain digestion are described in WO 94/29348 published and U.S. Pat. No. 4,342,566 (which are hereby incorporated by reference only for this teaching). Papain digestion of antibodies typically produces two identical antigen binding fragments, called Fab fragments, each with a single antigen binding site, and a residual Fc fragment. Pepsin treatment yields a fragment that has two antigen combining sites and is still capable of cross linking antigen.

The antibodies or antibody fragments may also include insertions, deletions, substitutions, or other selected modifications of particular regions or specific amino acids residues. These modifications can provide additional or improved function. For example, the removal or addition of acids capable of disulfide bonding may increase the bio-longevity of the antibody. In any case, the modified antibody or antibody fragment retains a desired bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr Opin. Biotechnol. 3:348-354, 1992).

The antibody or antibody fragment can be a mammalian antibody or an avian antibody. The antibody may be a human antibody or a humanized antibody. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86 95, 1991). Human antibodies (and fragments thereof) can also be produced using phage display libraries (Hoogenboom et al., J. Mol. Biol., 227:381, 1991; Marks et al., J. Mol. Biol., 222:581, 1991). The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551 255 (1993); Jakobovits et al., Nature, 362:255 258 (1993); Bruggermann et al., Year in Immunol., 7:33 (1993)).

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain that contains a portion of an antigen binding site from a non-human (donor) antibody integrated into the framework of a human (recipient) antibody. Fragments of humanized antibodies are also useful in the methods taught herein. Methods for humanizing non human antibodies are well known in the art. For example, humanized antibodies can be generated according to the methods of Winter and co workers (Jones et al., Nature, 321:522 525 (1986), Riechmann et al., Nature, 332:323 327 (1988), Verhoeyen et al., Science, 239:1534 1536 (1988)), by substituting rodent CDRs or CDR sequences for the corresponding sequences of a human antibody. Methods that can be used to produce humanized antibodies are also described in U.S. Pat. Nos. 4,816,567, 5,565,332, 5,721,367, 5,837,243, 5, 939,598, 6,130,364, and 6,180,377.

One embodiment of the inhibitor is an antibody that recognizes STIM1. The antibody may recognize any antigen on STIM1; one example described below employed an antibody against the N-terminal domain of STIM1. Other embodiments comprise an antibody that recognizes an extracellular domain of at least one of the ORAI1 protein and the ORAI3 protein. One embodiment of the inhibitor is an antibody that recognizes the extracellular domain of plasma membrane STIM1. Another embodiment of the inhibitor is an antibody that recognizes CPLA2. Such antibodies can be identified by methods such as phase display technology and used effectively as inhibitors, as described by R. Brissette, J. K. Prendergast, and N. I. Goldstein Current Opinions of Drug Discovery and Development 9:363-369 (2006); S. Main, R. Handy, J. Wilton, S. Smith, L. Williams, L. duFou, J. Andrews, L. A. Conroy, R. May, I. Anderson, and T. J. Vaughn, Journal of Pharmacology and Experimental Therapeutics, 319:1395-1404 (2006); A. M. Das, R. J. Flower, P. G. Hellewell, M. M. Teixeira, and M. Perretti, British Journal of Pharmacology 121:97-104 (1997) (each of which are incorporated by reference herein only to teach the identification, isolation, and use of antibodies with specific inhibitory activity). Similar approaches using monoclonal antibodies directed against plasma membrane STIM1 or ORAI proteins may also be efficacious.

3. Small Molecule Inhibitors

Various small molecules are known to inhibit the target molecules. One suitable example is LOE-908, which has been shown to block calcium entry through non-selective cation channels (see Miwa et al., Cardiovascular Drug Reviews, 18(1):61-72 (2000)). It has been unexpectedly discovered that LOE-908 specifically inhibits certain calcium channels, but not others. As described in the examples below, LOE-908 has been demonstrated to inhibit the activity of the ARC channel, while having no effect on the activity of the store-operated calcium channel. Those of ordinary skill in the art can acquire LOE-908 commercially in its hydrochloride form, for example from Tocris Bioscience of Ellisville, Mo.

Methyl-β-cyclodextrin has been unexpectedly discovered to exert a differential effect on ARC channel activity and SOCC activity. As described in the examples below, methyl-β-cyclodextrin completely suppresses ARC channel activity, while only reducing the activity of the SOCC to a minor degree. Methyl-β-cyclodextrin is known to reduce the concentration of cholesterol in the plasma membrane. While not wishing to be limited by any hypothetical mechanism of action, it is possible that methyl-β-cyclodextrin reduces the activity of both the SOCC channel and the ARC channel by removing cholesterol from cytosolic “lipid rafts” that are critical to the functioning of lipid molecules. The ARC channel may depend on the presence of lipid rafts for proper functioning. In accordance with this model, the inhibitor may be any substance that has a tendency to disrupt lipid rafts.

Descriptions of additional small molecule inhibitors are taught in the review by M. C. Meyer, P. Rastogi, C. S. Beckett, and J. McHowat, Current Pharmaceutical Design, 11:1301-1312 (2005) and A. A. Farooqui, W. Y. Ong, and L. A. Horrocks, Pharmacological Reviews 58:591-620 (2006) (incorporated herein by reference only to teach these inhibitors). These include arachidonyl trifluoromethyl ketone (AACOCF3) and methyl arachidonyl fluorophosphonate (MAFP).

Another example of a class of small molecule inhibitors is benzenesulfonamide and its piperidine derivatives, which potently inhibit heart CPLA2 activity at a low IC₅₀, as described by H. Oinuma, T. Takamura, T. Hasegawa, K. Nomoto, T. Naitoh, Y. Daiku, S. Hamano, H. Kakisawa, and N. Minami, Journal of Medicinal Chemistry, 34:2260-2267 (1991) (incorporated herein by reference only for the purpose of teaching these inhibitors). Intravenous injections of these inhibitors protect rats against ischemic damage in acute myocardial infarction. These compounds are metabolically stable having a half-life of 1-2 hours in vivo and are well tolerated in experimental animals.

Another exemplary class of inhibitors is the long-chain fatty acid 2-oxoamides of γ-aminobutyric acid and γ-norleucine 2-oxoamide, as described by G. Kokotos, D. A. Six, V. Loukas, T. Smith, V. Constantinou-Kokotou, D. Hadjipavlou-Litina, S. Kotsovolou, A. Chiou, C. C. Beltzner, and E. A. Dennis, Journal of Medicinal Chemistry, 47:3615-3628 (2004) (incorporated herein by reference only to teach these inhibitors). Kokotos et al. describe the in vivo administration of these novel CPLA2 inhibitors to animals, and the ability of these compounds to suppress rat paw carrageenan-mediated edema.

Another class of small-molecule inhibitors of CPLA2 activity are aristolochic acid (8-methoxy-6-nitrophenanthro[3,4-d][1,3]dioxole-5-carboxylic acid) and amylcinnamoyl-anthranilic acid. As shown in the examples below, these compounds, which inhibit CPLA2 activity, also effectively inhibit after-depolarization.

Another exemplary class of inhibitors are pyrrolidine-containing compounds described by K. Seno, T. Okuno, K. Nishi, Y. Murakami, F. Watanabe, T. Matsuura, M. Wada, Y. Fujii, M. Yamada, and T. Ogawa, Medicinal Chemistry, 43:1041-1044 (2000) (incorporated herein by reference only to teach this class of inhibitors). These compounds inhibit CPLA2 in vitro, and many structural variants (including pyrrolidine-1) have strong effects. These compounds are known to reduce skin inflammation in mouse models of atopic dermatitis and so are well tolerated in animals. This class of compounds is commercially available from sources like Cayman Chemicals under the trade name of RSC-3388.

Another CPLA2 inhibitor is a compound designated BMS-229724 (4-[4-[2-[2-[bis(4-chlorophenyl)methoxy]ethyl-sulfonyl]ethoxy]phenyl]-1,1,1-trifluoro-2-butanone). This compound is a selective inhibitor of CPLA2, with an IC₅₀ of 2.8 μM (J. R. Burke, L. B. Davem, P. L. Stanley, K. R. Gregor, J. Banville, R. Remillard, J. W. Russell, P. J. Brassil, M. R. Witmer, G. Johnson, J. A. Tredup, and K. M. Tramposch, Journal of Pharmacology and Experimental Therapeutics, 298:376-385 (2001)—incorporated herein by reference only to teach this inhibitor). In this study a solution of BMS-229724 in water/cremophor/ethanol (75:12.5:12.5) at 2.0 mg/ml was administered to rats through a cannula as an intravenous infusion or as an intraportal infusion of 1-4 mg/kg. A third group of rats was administered BMS-229724 (4.0 mg/ml in water/cremophor/ethanol, 75:12.5:12.5) by oral gavage at 20 mg/kg. This compound was shown to inhibit CPLA2 activity in vivo as manifested by a suppression of skin inflammation.

Another class of CPLA2 inhibitors are the substituted indole compounds taught in U.S. Pat. No. 6,797,708 and taught in Z. Ni, N. Okeley, B. Smart, and M. H. Gelb, Journal of Biological Chemistry, 281:16245-16255 (2006)(both of which are incorporated herein by reference to teach these inhibitors). One such compound designated “Wyeth-1” is well tolerated by animal models (M. I. Patel, J. Singh, M. Niknami, C. Kurek, M. Yao, S. Lu, F. Maclean, N. J. C. King, M. H. Gelb, K. F. Scott, P. J. Russell, J. Boulas, and Q. Dong, Clinical Cancer Research, 14:8070-8079 (2008)—which is incorporated by reference herein only to teach the administration of such compounds to animals).

D. Preventing and/or Reversing After-Depolarization

The disclosure provides methods of preventing or reversing an after-depolarization event in a cardiac myocyte. The method comprises contacting the cardiac myocyte with an active compound in an amount sufficient to achieve at least one of preventing an after-depolarization that might otherwise occur, or reversing an after-depolarization in a myocyte that has recently experienced an after-depolarization (this amount constituting an “effective amount” in this context). The active compound may be any active compound disclosed herein. The method may be performed in vitro or in vivo. When the method is performed in vivo, the cardiac myocyte will be a cell of a living subject. The in vivo myocyte may, for example, be a component of a cardiac muscle or a heart in a living subject. The in vitro myocyte may be a component of an isolated muscle, a portion of a heart, or an intact heart; in some embodiments the muscle, portion, or heart may be perfused or superfused. The disclosure also provides a use for any of the active compounds disclosed herein for the prevention or reversal of an after-depolarization in a myocyte, comprising the method described above. The disclosure also provides a use for any of the active compounds disclosed herein for the production of a medicament for the prevention or reversal of an after-depolarization in a myocyte. The disclosure also provides a use for any of the active compounds disclosed herein for the production of a pharmaceutical for the prevention or reversal of an after-depolarization in a myocyte.

Embodiments of the method and use comprise inhibiting the activity of the ARC channel or another target molecule. In such embodiments, the active compound will be present in concentrations sufficient to achieve such inhibition. Such concentrations can be determined by those of ordinary skill in the art, for example by use of the cardiac muscle assay described below. For example, the active compound may be present in a concentration of about 10 μM. As described in the examples below, the compound LOE-908 effectively reduces after-depolarization at concentrations as low as 1 μM, and at 20 μM no after-depolarization is observed. Accordingly, the concentration of LOE-908 in the current methods and uses may be at least 1 μM, at least 5 μM, at least 10 μM, at least 20 μM, at least 40 μM, any range interval therein, and about any of the foregoing concentrations. As described in the examples below, antibodies to Stim1 effectively eliminate after-depolarization at concentrations as low as 3 μg/mL. Accordingly, the concentration of antibodies to Stim1 may be 3 μg/mL, at least 3 μg/mL, or about any of the foregoing concentrations. As described in the examples below, methyl-β-cyclodextrin effectively eliminates after-depolarization at concentrations as low as 10 mM. Accordingly, the concentration of methyl-β-cyclodextrin may be 10 mM, at least 10 mM, or about any of the foregoing concentrations. As described in the examples below, aristolochic acid and amylcinnamoyl-anthranilic acid (ACA) suppress after-depolarization; ACA suppresses after-depolarization at concentrations as low as 10 μM. The examples below demonstrate that ACA has an observed IC50 of 10±4.5 μM and aristolochic acid has an observed IC₅₀ of 31±5 μM. Accordingly, the concentration of ACA in the current methods and uses may be equal to or greater than 5.5 μM, 10 μM, 14.5 μM, 30 μM, 50 μM, or about any of the foregoing ranges or concentrations.

### E. Pharmaceutical Compositions

The disclosure provides pharmaceutical compositions for the treatment and prevention of cardiac arrhythmia. The pharmaceutical compositions of the disclosure may be used in the treatment and prevention methods of the present disclosure. The pharmaceutical compositions disclosed may comprise any of: an active compound, a derivative of an active compound, a prodrug of an active compound, and a pharmaceutically acceptable salt of any of the foregoing. Some embodiments of the pharmaceutical composition comprise a derivative of an active compound, a pharmaceutically acceptable salt of an active compound, or a prodrug thereof. Some embodiments of the pharmaceutical composition further comprise a pharmaceutically acceptable carrier. Examples of such carriers and methods of formulation may be found in Remington: The Science and Practice of Pharmacy (20^{th} Ed., Lippincott, Williams & Wilkins, Daniel Limmer, editor.)

The disclosure also provides a use for any of the active compounds disclosed herein to produce a pharmaceutical composition. In a specific embodiment, the active compound is an inhibitor of CPLA2 activity. In an additional specific embodiment, the active compound is an inhibitor of the ARC channel. In a further specific embodiment, the active compound is an inhibitor of the SOCC.

Furthermore, the active compound may be present in a safe amount. A “safe” amount is an amount that has been determined to pose no potential negative effects to the subject, or to pose potential negative effects to the subject that are warranted given the benefits to be gained by administration. In some embodiments of the composition, the safe amount will be an amount that is determined to be unlikely to pose an observable negative effect on the subject. Such an amount may be the “no observable adverse effect level” or the “lowest observable adverse effect level.” The safe amount may also be below a known lowest lethal dose (LD₀). However, the safe amount may be associated with significant side effects or even a significant probability of lethality, if the benefits of administration are determined by a medical professional to offset the negative effects. Lower amounts typically have the advantage of causing fewer side effects or side effects of lesser severity. Higher amounts typically have the advantage of providing greater therapeutic effect. Such amounts are typically published in the results of clinical trials or in publically available regulatory rulemakings, and are often specific to the drug, the subject, and the disease. The principles underlying methods of measuring and characterizing the safety of chemical substances are described in C. D. Klaassen and D. L. Eaton, “Chapter 2: Principles of Toxicology” in Toxicology (1991) M. O. Amdur, J. Doull, and C. D. Klaassen, eds., which is incorporated by reference herein only for this teaching. In some embodiments of the pharmaceutical composition, the active compound is present in an amount that is both safe and effective.

To form a pharmaceutically acceptable composition suitable for administration, such compositions will contain a therapeutically effective amount of a compound(s). A therapeutically effective amount of the active compound will depend on numerous factors capable of being ascertained by those skilled in the art. In some embodiments, the therapeutically effective amount will be sufficient to achieve an extracellular concentrations disclosed as suitable to reverse or prevent after-depolarization in myocytes.

Such compositions are administered to a subject in amounts sufficient to deliver a therapeutically effective amount of the active compound so as to be effective in the treatment and prevention methods disclosed herein. The therapeutically effective amount may vary according to a variety of factors such as, but not limited to, the subject's condition, weight, sex and age. Other factors include the mode and site of administration. The pharmaceutical compositions may be provided to the subject in any method known in the art. Exemplary routes of administration include, but are not limited to, subcutaneous, intravenous, topical, epicutaneous, oral, intraosseous, intramuscular, intranasal and pulmonary. The compositions of the present disclosure may be administered only one time to the subject or more than one time to the subject. Furthermore, when the compositions are administered to the subject more than once, a variety of regimens may be used, such as, but not limited to, one per day, once per week, once per month or once per year. The compositions may also be administered to the subject more than one time per day. The therapeutically effective amount of the nucleic acid molecules and appropriate dosing regimens may be identified by routine testing in order to obtain optimal activity, while minimizing any potential side effects. In addition, co-administration or sequential administration of other agents may be desirable.

The compositions of the present disclosure may be administered systemically, such as by intravenous administration, or locally such as by subcutaneous injection or by application of a paste or cream.

The compositions of the present disclosure may further comprise agents which improve the solubility, half-life, absorption, etc. of the compound(s). Furthermore, the compositions of the present disclosure may further comprise agents that attenuate undesirable side effects and/or or decrease the toxicity of the compounds(s). Examples of such agents are described in a variety of texts, such as, but not limited to, Remington: The Science and Practice of Pharmacy (20^{th} Ed., Lippincott, Williams & Wilkins, Daniel Limmer, editor).

The compositions of the present disclosure can be administered in a wide variety of dosage forms for administration. For example, the compositions can be administered in forms, such as, but not limited to, tablets, capsules, sachets, lozenges, troches, pills, powders, granules, elixirs, tinctures, solutions, suspensions, elixirs, syrups, ointments, creams, pastes, emulsions, or solutions for intravenous administration or injection. Other dosage forms include administration transdermally, via patch mechanism or ointment. Further dosage forms include formulations suitable for delivery by nebulizers or metered dose inhalers. Any of the foregoing may be modified to provide for timed release and/or sustained release formulations.

In the present disclosure, the pharmaceutical compositions may further comprise a pharmaceutically acceptable carrier. Such carriers include, but are not limited to, vehicles, adjuvants, surfactants, suspending agents, emulsifying agents, inert fillers, diluents, excipients, wetting agents, binders, lubricants, buffering agents, disintegrating agents and carriers, as well as accessory agents, such as, but not limited to, coloring agents and flavoring agents (collectively referred to herein as a carrier). Typically, the pharmaceutically acceptable carrier is chemically inert to the active compounds and has no detrimental side effects or toxicity under the conditions of use. The pharmaceutically acceptable carriers can include polymers and polymer matrices. The nature of the pharmaceutically acceptable carrier may differ depending on the particular dosage form employed and other characteristics of the composition.

For instance, for oral administration in solid form, such as but not limited to, tablets, capsules, sachets, lozenges, troches, pills, powders, or granules, the compound(s) may be combined with an oral, non-toxic pharmaceutically acceptable inert carrier, such as, but not limited to, inert fillers, suitable binders, lubricants, disintegrating agents and accessory agents. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, and the like. Disintegrators include, without limitation, starch, methyl cellulose, agar, bentonite, xanthum gum and the like. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid as well as the other carriers described herein. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

For oral liquid forms, such as but not limited to, tinctures, solutions, suspensions, elixirs, syrups, antibodies and small molecules of the present disclosure can be dissolved in diluents, such as water, saline, or alcohols. Furthermore, the oral liquid forms may comprise suitably flavored suspending or dispersing agents such as the synthetic and natural gums, for example, tragacanth, acacia, methylcellulose and the like. Moreover, when desired or necessary, suitable and coloring agents or other accessory agents can also be incorporated into the mixture. Other dispersing agents that may be employed include glycerin and the like.

Formulations suitable for parenteral administration include aqueous and non-aqueous, isotonic sterile injection solutions, which can contain anti-oxidants, buffers, bacteriostats, and solutes that render the formulation isotonic with the blood of the patient, and aqueous and non-aqueous sterile suspensions that can include suspending agents, solubilizers, thickening agents, stabilizers, and preservatives. The active compound may be administered in a physiologically acceptable diluent, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as, but not limited to, a soap, an oil or a detergent, suspending agent, such as, but not limited to, pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include polyethylene sorbitan fatty acid esters, such as sorbitan monooleate and the high molecular weight adducts of ethylene oxide with a hydrophobic base, formed by the condensation of propylene oxide with propylene glycol, oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkylbeta-aminopropionates, and 2-alkylimidazoline quaternary ammonium salts, and (e) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17.

Topical dosage forms, such as, but not limited to, ointments, creams, pastes, emulsions, containing the antibodies and small molecules of the present disclosure, can be admixed with a variety of carrier materials well known in the art, such as, e.g., alcohols, aloe vera gel, allantoin, glycerine, vitamin A and E oils, mineral oil, PPG2 myristyl propionate, and the like, to form alcoholic solutions, topical cleansers, cleansing creams, skin gels, skin lotions, and shampoos in cream or gel formulations. Inclusion of a skin exfoliant or dermal abrasive preparation may also be used. Such topical preparations may be applied to a patch, bandage or dressing for transdermal delivery or may be applied to a bandage or dressing for delivery directly to the site of a wound or cutaneous injury.

The active compound of the present disclosure can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. Such liposomes may also contain monoclonal antibodies to direct delivery of the liposome to a particular cell type or group of cell types.

The active compound may also be coupled with soluble polymers as targetable drug carriers. Such polymers can include, but are not limited to, polyvinyl-pyrrolidone, pyran copolymer, polyhydroxypropylmethacryl-amidephenol, polyhydroxyethylaspartamidephenol, or polyethyl-eneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

### F. Assays to Detect an Anti-Arrhythmic Agent

An assay is provided to detect an anti-arrhythmic agent. Such agents may be useful as active agents included in pharmaceutical compositions or may be used to treat and/or prevent the disease states and conditions disclosed herein. Such compounds may then be further tested in appropriate systems (such as, but not limited to, animal models) to determine the activity of the identified compounds.

1. Whole-Cell and Cell-Free Assays

In an embodiment of the assay, a candidate compound is screened to determine whether it modulates or inhibits the ARC channel. Some embodiments of the assay are an assay to identify a modulator of a molecule that stimulates the ARC channel, such as CPLA2. The assay comprises contacting a candidate agent to an ARC channel or to a molecule that stimulates ARC channel activity (“assay target”) and measuring the activity of the ARC channel or the assay target. In general, such screening methods comprises the steps of providing an assay system (as described in more detail below) that expresses the assay target, introducing into the assay system a candidate compound to be tested and determining whether the candidate compound binds to the assay target, inhibits the activity of the ARC channel, or modulates the activity of a polypeptide regulated by the ARC channel. Such inhibition or modulation may act directly on the activity of the assay target or may be an inhibition or modulation of expression. In specific embodiments of the assay the assay target is the ARC channel, a voltage-independent calcium channel, SOCC, phospholipase, a phospholipase ligand, CPLA2, and a CPLA2 ligand.

Another embodiment of the assay comprises contacting a cell with a candidate agent, said cell comprising an assay target, and measuring the activity of the ARC channel or the assay target. The cell may be any cell comprising the assay target, although the use of cardiac myocytes has many advantages. The activity of the ARC channel or assay target in cardiac myocytes will be more closely correlated with the activity in a heart of an intact subject.

Candidate compounds are identified using a variety of assays, such as, but not limited to, assays that employ cells which express the assay target on the cell surface (cell-based assays) or in assays with isolated assay target (cell-free assays). The various assays can employ a functional variant of the assay target.

Where the assay involves the use of a whole cell, the cell may either naturally express the assay target or may be genetically modified to express the same. In the latter case, cells can be modified to express assay target channel through conventional molecular biology techniques, such as by infecting the cell with a virus comprising a nucleic acid encoding one or more polypeptide subunits of the assay target, wherein the assay target is expressed in the cell following infection. The cell can also be a prokaryotic or eukaryotic cell that has been transfected with a nucleotide sequence encoding one or more polypeptide subunits of the assay target. In the foregoing, full length polypeptides, derivatives of one or more polypeptide subunits of the assay target, functional variants, fragments or fusion proteins containing at least a part of such polypeptide may be used.

The assay can be a binding assay entailing direct or indirect measurement of the binding of a test compound to at least one of: a polypeptide subunit of the assay target, a complex of two or more subunits of the assay target, and the assembled assay target. The assay can also be an activity assay entailing direct or indirect measurement of the activity of the ARC channel. The assay can also be an expression assay entailing direct or indirect measurement of the expression of mRNA or protein of an assay target.

The various screening assays may be combined with an in vivo assay entailing measuring the effect of the test compound on the symptoms the disease states and conditions discussed herein. In such an embodiment, the compounds may be evaluated to determine if they impact a parameter associated with the action of the ARC channel. Such parameters include, but are not limited to, determining intracellular calcium concentrations upon exposure to an external medium comprising calcium and arachidonate. An example of such an ARC channel activity assay is the Fura-2-acetoxymethyl ester assay (“Fura-2AM assay”).

In some embodiments of the Fura-2AM assay, non-excitable cells can be used; in some embodiments, myocytes (including primary myocytes) can be used, isolated from any suitable source, including a rat heart or a heart from another mammal. In further embodiments, cultured immortalized myocytes (such as HL-1 myocytes) can be used. Immortalized myocytes have the advantage of allowing easy reproduction of the assay. For ease of observation, the cells may be adhered to a translucent surface, such as a slide coverslip. Adhesion may be achieved for example by growing on or seeding the cells onto the translucent surface in appropriate culture medium. When these cells have grown to an appropriate density or attached to the translucent surface, they can be exposed to Fura-2-acetoxymethyl ester calcium indicator dye (acetoxymethyl 2-[5-[bis[(acetoxymethoxy-oxo-methyl)methyl]amino]-4-[2-[2-[bis[(acetoxymethoxy-oxo-methyl)methyl]amino]-5-methyl-phenoxy]ethoxy]benzofuran-2-yl]oxazole-5-carboxylate) in HEPES-buffered physiological salt solution or an equivalent solution at 2 μM and at 37° C. The Fura-2AM indicator can be obtained commercially from Sigma Chemical Co. of St. Louis, Mo. or other distributors.

Cell exposure to the Fura-2AM can last up to 30 min. The cells are then washed to remove excess Fura-2AM and incubated an additional 30 mM to allow de-esterification of intracellular Fura-2AM.

The Fura-2-loaded cells then can be placed on an inverted epifluorescence microscope configured to allow the rapid addition and removal of the external solution bathing the coverslip (for example the Nikon 200 or a Nikon Diaphot TE300, Nikon Corporation, Tokyo, Japan). The sample is then exposed to alternating wavelengths of light at 340 and 380 nm. Alternation of these wavelengths can be controlled by a high-speed monochromator (such as the TILL Polychrome IV or a 340HT15 and 380HT15 filter changer from Sutter Instrument, Novato, Calif.). Intracellular Fura-2 that is exposed to light at 340 and 380 nm will reemit at 500±45 nm. Light in the reemission region is then measured. The reemitted light can be measured by various instruments, such as a cooled charge-coupled device camera, like an Astrocam camera. The reemitted light may be acquired with a typical exposure time of 10-15 ms, and data points are acquired one per second. These are stored on computer-readable media using software known to those skilled in the art, for example the Ultraview software package from Perkin Elmer of Waltham, Mass.

To assess ARC channel activity, during observation of the cells' reemission spectrum, the cells are exposed for several minutes to medium that is nominally free of calcium and contains up to 30 μM arachidonic acid. After 10-15 minutes, cells are exposed to medium containing up to 2 mM calcium and 30 μM arachidonic acid for a period of up to 15 minutes.

Any increase in cell calcium measured as an increase in Fura-2 fluorescence when cells are exposed to medium containing calcium and arachidonate is a direct measure of the ARC channel activity. Any agent that decreases the peak calcium entry under these conditions or the total calcium entry under these conditions is designated an ARC channel inhibitor.

In one embodiment, the present disclosure provides assays for screening candidate or test compounds which bind to or modulate the activity of a membrane-bound (cell surface expressed) form of the assay target. Such assays can employ full-length polypeptide subunit of the assay target (alone or complexed with one or more additional subunits), a functional variant thereof, a biologically active fragment thereof, or a fusion protein which includes all or a portion of the subunit. As described in greater detail below, the test compound can be obtained by any suitable means (such as from conventional compound libraries). The assay target, subunit, or complexed subunits may be expressed in a whole cell or in a liposome, micelle or similar lipid containing structure.

Determining the ability of the test compound to bind to a membrane-bound form of the assay target, subunit, or complexed subunits can be accomplished, for example, by coupling the test compound with a radioisotopic, colorimetric, spectroscopic, or enzymatic label such that binding of the test compound to the assay target-expressing cell can be measured by detecting the labeled compound in a complex.

For example, the test compound can be labeled with ¹²⁵I, ³⁵S, ¹⁴C, or ³H, either directly or indirectly, and the radioisotope detected by direct counting of radio-emission or by scintillation counting. Alternatively, the test compound can be enzymatically labeled with, for example, horseradish peroxidase, alkaline phosphatase, or luciferase, and the enzymatic label detected by determination of conversion of an appropriate substrate to product. The test compound can also be labeled with a fluorescent molecule or an antigenic molecule (such as the FLAG motif).

In a competitive binding format, the assay comprises contacting the assay target-expressing cell or liposome with a known compound which binds to the assay targetto form an assay mixture, contacting the assay mixture with a test compound, and determining the ability of the test compound to interact with assay target-expressing cell, wherein determining the ability of the test compound to interact with the assay target-expressing cell comprises determining the ability of the test compound to preferentially bind the ARC channel-expressing cell as compared to the known compound.

In another embodiment, the assay is a cell-based assay comprising contacting a cell expressing a membrane-bound form of the assay target (a full-length polypeptide subunit of the ARC channel, a biologically active fragment thereof, a fusion protein which includes all or a portion thereof, or two or more of the foregoing in complex) expressed on the cell surface with a test compound and determining the ability of the test compound to inhibit the activity of the membrane-bound form of the assay target. Determining the ability of the test compound to inhibit the activity of the membrane-bound form of the assay target can be accomplished by any method suitable for measuring the activity of the assay target.

Determining the ability of the test compound to modulate the activity of the assay target can be accomplished, for example, by determining the ability of the assay targetto bind to or interact with a ligand with which the assay target is known to bind. The ligand can be a molecule with which the assay target binds or interacts in nature; examples include a molecule on the surface of a cell which expresses the assay target, a molecule on the surface of a second cell, a molecule in the extracellular milieu, a molecule associated with the internal surface of a cell membrane, or a cytoplasmic molecule. The ligand can be a component of a signal transduction pathway which facilitates transduction of an extracellular signal (for example, a signal generated following binding of an ARC channel ligand) through the cell membrane and into the cell. The ligand can be, for example, a second intracellular protein which has catalytic activity or a protein which facilitates the association of signaling molecules with the assay target or the activation of signaling molecules downstream of the assay target.

Determining the ability of the assay targetto bind to or interact with a ligand can be accomplished by one of the methods described above for determining direct binding. In one embodiment, determining the ability of a compound of the invention to bind to or interact with a ligand can be accomplished by determining the activity of the ligand.

The present disclosure also includes cell-free assays. Such assays involve contacting a form of the assay target with a test compound and determining the ability of the test compound to bind to the assay target or to inhibit the assay target. Binding of the test compound to the assay target can be determined either directly or indirectly as described above. Regulation of the assay target activity can be determined as discussed above.

In one embodiment, the assay includes contacting a cell free system containing the assay target or with a known compound to form an assay mixture, contacting the assay mixture with a test compound, and determining the ability of the test compound to interact with the assay target, wherein determining the ability of the test compound to interact with the assay target comprises determining the ability of the test compound to preferentially bind the assay target as compared to the known compound.

The cell-free assays of the present disclosure are amenable to use of either a membrane-bound form of the assay target or a soluble fragment thereof. In the case of cell-free assays comprising the membrane-bound form of the polypeptide, it may be desirable to utilize a solubilizing agent such that the membrane-bound form of the polypeptide is maintained in solution. Examples of such solubilizing agents include but are not limited to non-ionic detergents such as n-octylglucoside, n-dodecylglucoside, n-dodecyhnaltoside, octanoyl-N-methylglucamide, decanoyl-N-methylglucamide, Triton X-100, Triton X-114, Thesit, Isotridecypoly (ethylene glycol ether) n, 3-[(3-cholamidopropyl) dimethylamminio]-1-propane sulfonate (CHAPS), 3-[(3-cholamidopropyl) dimethylamminio]-2-hydroxy-1-propane sulfonate (CHAPSO), or N-dodecyl=N,N-dimethyl-3-ammonio-1-propane sulfonate.

In various embodiments of the above assay methods, it may be desirable to immobilize an assay target comprising multiple subunits to facilitate separation of complexed from uncomplexed forms of a polypeptide subunits, as well as to accommodate automation of the assay. Binding of a test compound to an assay target subunit (alone or complexed to another assay target subunit), or interaction of the assay target with a polypeptide regulated by the assay target in the presence and absence of a test compound, can be accomplished in any vessel suitable for containing the reactants.

Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase (GST) fusion proteins can be adsorbed onto glutathione sepharose beads or glutathione derivatized microtitre plates, which are then combined with the test compound and the mixture incubated under conditions conducive to complex formation (for example at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of binding or activity of the assay target can be determined using standard techniques.

The screening assay can also involve monitoring the expression of the assay target. For example, regulators of expression of the assay target or one of its subunits can be identified in a method in which a cell is contacted with a test compound and the expression of the assay target or subunit or mRNA encoding the foregoing in the cell is determined. The level of expression of polypeptide or mRNA in the presence of the test compound is compared to the level of expression of in the absence of the test compound. The test compound can then be identified as a regulator of expression of the assay target based on this comparison. For example, when expression of polypeptide or mRNA is decreased in the presence of the test compound relative to its absence, the test compound is identified as an inhibitor of polypeptide or mRNA expression. The level of polypeptide or mRNA expression in the cells can be determined by methods described below.

The level of mRNA or polypeptide expression in the cells can be determined by methods well known in the art for detecting mRNA or polypeptide. Either qualitative or quantitative methods can be used. The presence of a polynucleotide encoding an assay target subunit can be determined, for example, using a variety of techniques known in the art, including Northern blots, Southern blots, microarray testing, and PCR techniques (including but not limited to real-time PCR). The presence of polypeptide products of the assay target polynucleotide can be determined, for example, using a variety of techniques known in the art, including immunochemical methods such as radio-immunoassay, Western blotting, microarray testing, and immunohistochemistry. Alternatively, polypeptide synthesis can be determined in vivo, in a cell culture, or in an in vitro translation system by detecting incorporation of labeled amino acids into a subunit of the assay target.

Such screening can be carried out either in a cell-free assay system or in an intact cell. Any cell which expresses the assay target polynucleotide can be used in a cell-based assay system. The polynucleotide can be naturally occurring in the cell or can be introduced using techniques such as those described above. Either a primary culture or an established cell line can be used.

The assay may further comprise contacting the cell or assay target with an arrhythmic agent. The arrhythmic agent may be any agent that causes arrhythmia by modulating the assay target or a pathway that in turn modulates the assay target. One suitable arrhythmic agent is Anemonia sulcata toxin (ATX II) or a functional variant thereof, which has been unexpectedly discovered to induce arrhythmia and after-depolarization at a concentration of about 5-60 nM through interference with the functioning of the ARC channel (see the examples below).

Another suitable arrhythmic agent is 2-APB. Other examples arrhythmic agents that may be used include: inhibitors of potassium channels, and the combination of inhibitors of potassium channels and appropriate arrhythmogenic ligands. Such arrhythmogenic ligands may be an alpha-adrenergic agonist, such as methoxamine.

2. Cardiac Muscle Assay

Embodiments of the methods disclosed involve exposing viable cardiac muscle to an arrhythmic agent under conditions in which the cardiac muscle would otherwise display normal electro-mechanical activity. Embodiments of the assay comprise: superfusing an isolated cardiac muscle with a physiologically suitable buffer, introducing pacing stimulus to the isolated cardiac muscle, exposing the isolated cardiac muscle to a concentration of an arrhythmic agent that acts on the ARC channel or pathway, exposing the isolated cardiac muscle to a concentration of a suspected anti-arrhythmia agent, and measuring at least one of mechanical activity and electrical activity of the isolated cardiac muscle. In some embodiments of the assay, the isolated cardiac muscle may be replaced with isolated cardiac myocytes, cultured cardiac myocytes or Purkinje fibers/cells.

Some embodiments of the method comprise superfusing an isolated cardiac muscle with a physiologically suitable buffer, introducing pacing stimulus to the isolated cardiac muscle, exposing the isolated cardiac muscle to a concentration of the arrhythmic agent, removing pacing stimulus from the isolated cardiac muscle, measuring the rate of mechanical activity in the isolated cardiac muscle, exposing the isolated cardiac muscle to a concentration of the candidate anti-arrhythmic agent, and measuring at least one of mechanical activity and electrical activity of the isolated cardiac muscle. The isolated cardiac muscle may be a non-automatic cardiac muscle.

In further embodiments of the method, the isolated cardiac muscle is a left atrial appendage. In further embodiments of the method, the isolated cardiac muscle is a right ventricular muscle strip. Other embodiments include perfused whole hearts, papillary muscles, isolated cardiac myocytes, cultured cardiac myocytes, Purkinje fibers/cells or other muscle preparations. In further embodiments of the method, the buffer comprises Krebs-Henseleit (KH) perfusate. In further embodiments of the method, the method further comprises inducing high concentrations of intracellular calcium. In further embodiments, the concentration of arrhythmic agent is sufficient to induce after-depolarization or arrhythmia in the cardiac muscle.

Some embodiments, of the method comprise: (a) superfusing or perfusing an isolated cardiac muscle in KH perfusate at an approximately physiological temperature, the isolated cardiac muscle selected from the group consisting of: left atrial appendage, right ventricular muscle strip, intact heart or papillary muscle and a combination thereof; (b) pacing the muscle at an approximately sub-physiological rate; (c) exposing the muscle to an arrhythmia-inducing agent; (d) increasing the internal calcium of the muscle either prior to step (b) or following step (e); (e) observing an occurrence of arrhythmia, such as but not limited to an after-depolarization event; (f) increasing pacing to an approximately physiological rate, the increasing step following step (e); and (g) exposing the muscle to a candidate anti-arrhythmic agent. Increasing internal calcium further comprises contacting the muscle with a substance capable of inducing an increase in cytoplasmic calcium, as described in the previous paragraph.

In further embodiments the muscle is exposed to the arrhythmia-inducing agent. The arrhythmia-inducing agent may be an agent that induces after-depolarization in the myocyte. In further embodiments the arrhythmia-inducing agent is ATX-II, a functional variant of ATX-II, a structurally-related compound, a functionally related compound, or a combination thereof. In further embodiments, the candidate anti-arrhythmic agent is a previously established anti-arrhythmic agent employed as a positive control. In some embodiments the arrhythmic agent is 2-APB. Further embodiments may employ any arrhythmic agent that is discussed above in the whole-cell and cell-free assays. In an alternative embodiment the cells of the cardiac muscle have been genetically modified to enhance the activity of the ARC channel or a molecule that stimulates the ARC channel.

The cardiac muscle may be part of a functioning heart (in vivo or in vitro), but in certain embodiments it is isolated from a host animal. The muscle may be from any mammal, but it is preferably from a commonly used animal model or from a human. Commonly used animal models that can provide suitable cardiac muscle include but are not limited to Norway rat, cotton rat, mouse, cavy, cat, hamster, dog, gerbil, sheep, goat, rabbit, swine, monkey, or ape. If the animal source of the muscle is a commonly used animal model, it can be of any subspecies or breed. Such animals can be obtained from sources familiar to those skilled in the art. The animal source may be genetically modified or unmodified, according to the needs of the specific assay.

Isolated cardiac muscle can be removed from live or recently dead mammalian subjects by methods familiar to those skilled in the art. Preferably, the handling of the animals will conform to the standards set out in the Guide for the Care and Use of Laboratory Animals published by the U.S. National Institutes of Health (NIH Pub. No. 85-23, 1996).

In embodiments of the method, the isolated cardiac muscle is left atrial appendage (LAA), a cardiac papillary muscle, or right ventricular muscle strip (RVMS) isolated from the animal. However, other cardiac muscles may be used. The left atrium, papillary muscles, and the right ventricle have the advantage of having no automatic activity. The automatic activity of the right atrium, for example, could potentially interfere with observations of arrhythmia in the muscle. Such interference will not be present when left atrium or left ventricle is used. The LAA and RVMS have the additional advantage of being well understood systems and of requiring far less media than is required by other perfused muscle systems. This latter is an even greater advantage if costly agents are the subject of the assay, as large amounts of the agent will be required to maintain a steady concentration in large volumes of perfusate. If it is desired to study the effects of an agent on automatic activity, then the right atrium would then be preferable.

Cardiac muscle can be prepared by any method known by those skilled in the art. For example, animals can be injected with appropriate doses of anesthetics to achieve a deep plane of sedation. An appropriate dose of heparin can then be administered to these animals to prevent blood coagulation during all subsequent procedures. A bilateral thoracotomy can be performed and hearts then extirpated and placed in ice-cold KH buffer. Intact hearts would then be trimmed, mounted on a perfusion cannula and then perfused in a Langendorff or working heart-mode (one example of this approach is described in Balschi J A, et al., “Model systems for modulating the free energy of ATP hydrolysis in normoxically perfused rat hearts” J. Molecular and Cellular Cardiology 29:3123-3133 (1997), which is hereby incorporated by reference only for such teaching). Alternatively, appropriate muscles are surgically isolated from intact hearts and superfused in a standard muscle bath; these isolated muscles include left atrial appendage, right ventricular muscle strips (for examples of this approach see Grupp et al. “Isolated heart preparations perfused or superfused with balanced salt solutions” in Methods in Pharmacology vol. 5, Plenum Press: New York, pages 111-128 (1984), which is hereby incorporated by reference only for such teaching) or right or left ventricular papillary muscles (for examples of this approach, see Urthaler et al., “Estimates of beat to beat handling of activator calcium using measurements of [Ca2+]; in aequorin loaded ferret cardiac muscle” Cardiovascular Research 28: 40-46 (1994), which is hereby incorporated by reference only for such teaching).

If the assay is performed in vitro, the isolated muscle must be maintained in an appropriate muscle bath. An appropriate muscle bath must allow the isolated muscle to function mechanically during the assay. Such muscle baths include any such baths familiar to those skilled in the art. One example is KH buffer with added glucose (NaCl 118 mM, NaHCO₃ 27 mM, KCl 4.8 mM, MgSO₄ 1.2 mM, KH₂PO₄ 1.0 mM, CaCl₂ 1.8 mM, glucose 11.1 mM). The buffer must be at a suitable temperate, appropriate to allow functioning of the muscle, such as in one embodiment at about physiological temperature (about 37° C.). The assay may be performed at a sub-physiological temperature (such as 30° C.). The isolated muscle may be superfused with the buffer prior to the assay at an appropriate temperature, for example about 30° C.

Suitable muscle bath or buffer preferably contains adequate concentrations of potassium, sodium and chloride to permit ATX-II to act as an arrhythmia inducing agent. As an example, in KH buffer with added glucose, it has been observed that the ability of ATX-II to induce arrhythmia starts to decrease below around 10-50 nM.

In alternative embodiments, the cardiac muscle is a component of the heart of a live animal. The steps of the assay can be carried out in live animals by means familiar to those skilled in the art.

In one embodiment, animals are anesthetized and intubated, a pressure transducer is inserted into the femoral artery, a lead II ECG is obtained, and their intact beating hearts are exposed (see, for example, Huang J. et al., “Restitution properties during ventricular fibrillation in the in situ swine heart” Circulation 110:3161-3167 (2004), which is hereby incorporated by reference only for such teaching). Piezoelectric crystals then may be implanted into the ventricle to acquire local mechanical activity in these intact hearts (see, for example, Wolkowicz et al., “Sodium-calcium exchange in dog heart mitochondria: effects of ischemia and verapamil” American Journal of Physiology 244: H644-H651 (1983), which is hereby incorporated by reference only for such teaching).

Five instructive examples are given for the administration of ATX-II and functionally related compounds to these beating in vivo hearts.

First, a solution of concentrated 2-APB (10-50 μM), ATX II (10-70 nM), or functionally related compound suspended in DMSO may be placed directly onto the intact left atrium, left ventricle or other region of the beating heart as is experimentally desired. This can be accomplished using various approaches including a cotton swab, a cotton pad or using a dispenser such as a pipetter. Second, a solution of concentrated 2-APB, ATX-II or functionally related compound could be placed into an appropriate syringe and this solution can be injected directly into the ventricular endo-myocardium or mid-myocardium or into the atrial muscle wall. The needle used should be of an appropriate gauge so as not to compromise muscle integrity (see Scherf, D., “Studies on auricular tachycardia caused by aconitine administration.” Proceedings of the Society for Experimental Biology 64: 233-239 (1947), which is hereby incorporated by reference only for such teaching). Third, an experimentally important artery may be isolated and cannulated, and the perfused heart bed may then be infused with saline or blood solutions containing appropriate concentrations of ATX-II, 2-APB, or functionally related molecules.

Using these approaches, a stable focus of sporadic ectopic activity should be produced. Changes in in vivo heart electrical activity can be monitored through a lead II ECG and the appearance of ectopic depolarizations is the end point for this approach. Likewise, local or whole organ mechanical activity can be measured using the pressure transducer or the piezoelectric crystals, respectively (for example, see Wolkowicz et al., “Sodium-calcium exchange in dog heart mitochondria: effects of ischemia and verapamil” American Journal of Physiology 244: H644-H651 (1983), which is hereby incorporated by reference only for such teaching). The sporadic, ectopic electro-mechanical events will be either atrial or ventricular in origin depending on the point of ATX-II application.

The in vivo efficacy of anti-arrhythmic agents can then be tested by methods understood by those skilled in the art. The following three general methods for testing can be used by way of example. First, the test anti-arrhythmic compounds could be infused into the general circulation. Second, the test anti-arrhythmic compounds could be infused through a cannula into the specific muscle bed that had been made ectopic. Third, animals could be pre-treated with a test anti-arrhythmic agent in their drinking water or chow prior to the induction of sporadic or tachycardic arrhythmias. Under these in vivo conditions a candidate anti-arrhythmic agent is identified if the test agent suppresses sporadic or tachycardic ectopic activity and restores or maintains lead II ECG characteristics to normal.

In embodiments of the assay the muscle must be paced by any appropriate means familiar to those skilled in the art. If the muscle is intact in a living subject, or if the muscle includes a sinoatrial node, pacing may be provided by the sinoatrial node, or it may be provided externally (such as by a pacemaker). If the muscle is an isolated cardiac muscle, then some form of external pacing must be provided, including but not limited to the example of a pace-making device. The pacing rate may be approximately physiological, supra- or sub-physiological. The pacing rate may be greater than approximately physiological; however, if the assay is to detect an agent with properties of inducing tachycardia, then it is preferable that the pacing rate not significantly exceed an approximately physiological rate. Physiological rates of pacing vary between species, and roughly correspond to the animal's heart rate. Physiological pacing rates of different species of mammal are well understood by those skilled in the art. For example, the approximate heart rate of a healthy Norway rat is 330-480 beats/minute. The approximate heart rate of a healthy mouse is about 632 +/−51 beats per minute (adult) or 286 +/−56 beats per minute (newborn). The approximate heart rate of a healthy cavy is 240-300 beats per minute. The approximate heart rate of a healthy gerbil is 360 beats per minute. The approximate heart rate of a healthy hamster is 250-500 beats per minute. The approximate heart rate of a healthy Rhesus monkey is 120-180 beats per minute. The approximate heart rate of a healthy sheep or goat is 80-120 beats per minute. An animal's heart rate may vary greatly depending on its sex, age, state of activity, temperature, culture conditions (in vitro) or state of health, as is understood by those skilled in the art.

The rate of pacing may be varied according to the needs of the assay. In some embodiments, the muscle is paced at the same rate throughout. In some embodiments, the rate of pacing may be varied at certain points during the assay. In some embodiments, including but not limited to assays for agents that inhibit tachycardia, initial pacing occurs at approximately a sub-physiological rate, and the pacing rate is increased to an approximately physiological rate. In assays for tachycardia, the pacing rate may be increased after the appearance of tachycardia.

In embodiments of the assay an agent to induce arrhythmia will be introduced in the muscle. The arrhythmic agent may be chemical or physical. If the agent is chemical, it can be any chemical agent known by those skilled in the art to induce arrhythmia. The chemical agent will be introduced at a concentration sufficient to induce arrhythmia. If a certain type of arrhythmia is the subject of the assay (for example, triggered activity, tachycardia or any other type of arrhythmia), then the agent should be present at a concentration sufficient to produce the certain type of arrhythmia. One acceptable agent is ATX II, which is a sodium channel regulator. Other arrhythmic agents include inhibitors of potassium channels and activators of heart myocyte receptor signaling, as for example cited in S. Kaseda and D. P. Zipes, J. Cardiovascular Electrophysiology 1: 31-40 (1999) or J. Ben-David and D. P. Zipes, Circulation 78:1241-1250 (1988) (incorporated herein by reference only to teach such arrhythmic agents).

In embodiments of the assay the mechanical or electrical activity of the muscle is measured over a period of time beginning prior to the exposure of the muscle to a possible anti-arrhythmic agent and lasting at least until after the exposure of the muscle to a possible anti-arrhythmic agent. Mechanical and/or electrical activity can be measured by any suitable method, including but not limited to use of a force transducer or an oscilloscope. Such measurement should measure the rate of mechanical or electrical activity, and may optionally measure the force or amplitude of mechanical or electrical activity.

For example, isolated superfused muscles can be impaled with conventional glass microelectrodes of 10 to 30 MΩ resistance; these microelectrodes are filled with a solution of 3 M potassium chloride. Microelectrodes are routinely mounted on 30 μm silver-silver chloride spiral wire to allow for freedom of motion. Following the successful impalement of an individual myocyte, action potentials can be recorded with DC coupling at the center of the ring as the difference in voltage between the intracellular microelectrode and the extracellular silver-silver chloride reference electrode. Electrical signals are passed through a high-impedance capacitance-compensation preamplifier and are recorded on a personal computer at a sampling rate of ˜3 Hz. Subsequent data analysis of the action potentials of these muscles is performed using standard software (for an example of such a protocol, see Huang J et al., “Restitution properties during ventricular fibrillation in the in situ swine heart” Circulation 110:3161-3167 (2004), which is hereby incorporated by reference only for such teaching).

The electrical activity of intact hearts in surgically-prepared, open- or closed-chested animals can be obtained using a standard lead II ECG (for examples see Straeter-Knowlen I et al., “¹H NMR spectroscopic imaging of myocardial triglycerides in excised dog hearts subjected to 24 hours of coronary occlusion” Circulation 93: 1464-1470 (1996); and Huang J et al., “Restitution properties during ventricular fibrillation in the in situ swine heart” Circulation 110:3161-3167 (2004), which is hereby incorporated by reference only for such teaching).

Subsequent to exposure of the muscle to the arrhythmic agent, arrhythmia will be measured in the muscle. If normal rhythmic activity is restored subsequent to exposure of the muscle to the candidate anti-arrhythmic agent, or if ectopic activity is reduced in frequency or severity, then it can be concluded that the agent inhibits arrhythmia. If no change occurs, or if ectopic activity increases in frequency or severity, then it must be concluded that the agent does not inhibit arrhythmia.

3. Test Compounds

Suitable test compounds for use in the screening assays can be obtained from any suitable source, such as conventional compound libraries. The test compounds can also be obtained using any of the numerous approaches in combinatorial library methods known in the art, including: biological libraries, spatially addressable parallel solid phase or solution phase libraries, synthetic library methods requiring deconvolution, the “one-bead one-compound” library method and synthetic library methods using affinity chromatography selection. The biological library approach is limited to peptide libraries, while the other four approaches are applicable to peptide, non-peptide oligomer or small molecule libraries of compounds. Examples of methods for the synthesis of molecular libraries can be found in the art. Libraries of compounds may be presented in solution or on beads, bacteria, spores, plasmids or phage.

Computer modeling and searching technologies permit identification of compounds, or the improvement of already identified compounds, that can inhibit the ARC channel (either through expression or activity). Having identified such a compound, the active sites or regions are identified. Such active sites might typically be ligand binding sites. The active site can be identified using methods known in the art including, for example, from the amino acid sequences of peptides, from the nucleotide sequences of nucleic acids, or from study of complexes of the relevant compound or composition with its natural ligand.

In the latter case, chemical or X-ray crystallographic methods can be used to find the active site by finding where on the factor the complexed ligand is found. Next, the three dimensional geometric structure of the active site is determined. This can be done by known methods, including X-ray crystallography, which can determine a complete molecular structure. On the other hand, solid or liquid phase NMR can be used to determine certain intramolecular distances. Any other experimental method of structure determination can be used to obtain partial or complete geometric structures. The geometric structures may be measured with a complexed ligand, natural or artificial, which may increase the accuracy of the active site structure determined. If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, test compounds can be identified by searching databases containing compounds along with information on their molecular structure. Such a search seeks compounds having structures that match the determined active site structure and that interact with the groups defining the active site. Such a search can be manual, but is preferably computer assisted. Alternatively, these methods can be used to identify improved compounds known in the art or identified in one of the screening assays above. These compounds found may be used to inhibit the ARC channel.

The present disclosure also provides kits for carrying out any method of the present disclosure, which can contain any of the compounds and/or compositions disclosed herein or otherwise useful for practicing a method of the disclosure.

### G. Induction of After-Depolarization

The disclosure provides methods, compositions, and uses for inducing after-depolarization in a myocyte. In some embodiments the induction of after-depolarization results in arrhythmia in a cardiac muscle. Such methods, compositions, and uses have numerous utilities, including the production of models of after-depolarization and arrhythmia to facilitate research of these phenomena.

A method is provided of inducing after-depolarization in a myocyte comprising increasing the activity of the ARC channel or a molecule that stimulates the activity of the ARC channel (“upregulation target”). Specific examples of upregulation targets are an ARC channel subunit, a SOCC subunit, and CPLA2. Activity of the upregulation target can be accomplished in a variety of ways. One embodiment of the method comprises increasing the copy number of an upregulation target gene or subunit gene, as it is known in the art that increasing the copy number of a gene generally increases the expression of the polypeptide encoded by that gene, which in turn increases the overall activity of the polypeptide (or a protein or proteins of which the polypeptide is a component). A use of an upregulation target gene to induce after-depolarization in a cell is provided, comprising increasing the copy number of the upregulation target gene in the cell. In this context an “upregulation target gene” includes a gene for any polypeptide monomer that is a component of the upregulation target (referred to for brevity as an “upregulation target monomer”). Copy number can be increased by insertion of a polynucleotide that encodes an upregulation target polypeptide or that encodes a functional variant of an upregulation target polypeptide. In some cases, the upregulation target polypeptide may be the product of more than a single exon, in which case the polynucleotide may comprise an intron or additional exons that do not encode an upregulation target polypeptide. Additional copies of the upregulation target gene may be introduced into the myocyte by any method known in the art, including transfection, transformation, and transduction. Methods of transformation include those employing electroporation, sonoporation, lipoplexes, polyplexes, a gene gun, or other means of introducing a nucleic acid into a cell without a cellular or viral vector. In some embodiments of the method and use, the copy number of the upregulation target gene is increased in a multipotent, totipotent or pluripotent cell. In such embodiments the totipotent cell may proliferate to form a multicellular organism; the pluripotent cell may proliferate to form a germ layer within a multicellular organism; the multipotent cell may proliferate to form a tissue. Other embodiments of the method and use comprise introducing additional gene copies into a cell in vitro or into a terminally differentiated cell in vivo. Any embodiment of the method and use described in this paragraph can substitute a gene for an upstream regulator of the upregulation target for the upregulation target gene and achieve the same effect.

A method of inducing an after-depolarization in a cell is provided comprising coupling an upregulation target gene to a more active promoter. A use of a more active promoter to induce an after-depolarization is provided, comprising coupling an upregulation target gene to a more active promoter. In this context, a “more active promoter” refers to a promoter that is more active in allowing transcription of the ARC channel gene than is the native promoter. The increased promoter activity may be due to many factors, including an increased affinity for RNA polymerase II (RNA Pol II), a co-factor of RNA Pol II, or a transcription factor (including any of the known general transcription factors). Such increased affinity may be due to a difference in the binding energy between the promoter and RNA Pol II, its co-factor, or the transcription factor, or it may result from a reduced tendency of the promoter to interact with an inhibitor. Any embodiment of the method and use described in this paragraph can substitute a gene for an upstream regulator of the upregulation target for the upregulation target gene and achieve the same effect.

The disclosure provides a method of inducing an after-depolarization in a cell comprising contacting the cell with an agonist of the upregulation target. The disclosure also provides a use of an agonist of the upregulation target for inducing an after-depolarization in a cell comprising contacting the cell with the agonist of the upregulation target. The agonist will be present in a concentration sufficient to increase the activity of the upregulation target. Such agonists are known to those of ordinary skill in the art. Examples of agonists of the ARC channel include Stim1, arachidonic acid, metabolites of arachidonic acid, and other known lipid agonists of the ARC channel, as described above. Any embodiment of the method and use described in this paragraph can substitute a gene for an upstream regulator of the upregulation target for the upregulation target gene and achieve the same effect.

The disclosure provides a method of inducing an after-depolarization in a cell comprising modifying a site of inhibitor binding on the upregulation target to reduce the affinity of the site for an inhibitor. Such modification can be achieved by various means. For example, deletion of a fragment of the binding site (or deletion of the entire binding site) will disrupt inhibitor binding, as will insertion of a sufficiently long non-native peptide within the binding site. The binding site may be modified to reduce inhibitor binding by making non-conservative substitutions of one or more peptide residues in the binding site. After-depolarization may also be induced by making such a modification to a protein or polypeptide that is an upstream regulator of the upregulation target.

The disclosure provides a cell having increased ARC channel activity. Some embodiments of the cell are a myocyte, or more specifically a cardiac myocyte. Some embodiments of the cell comprise an additional copy of an ARC channel gene or a gene for an upstream regulator of the ARC channel (“upregulation target”). The upregulation targets discussed above for the method of producing an after-depolarization are also suitable for use in the cell. Other embodiments of the cell comprise a highly active promoter operably linked to an upregulation target gene or a gene for an upstream regulator of the ARC channel. Other embodiments of the cell comprise a gene for the upregulation target that encodes an upregulation target polypeptide that has been modified at a site of inhibitor binding on the upregulation target to reduce the affinity of the site for an inhibitor. Of course, the cell may comprise more than one of the foregoing elements to increase upregulation target activity. The cell may display after-depolarization more frequently than would an unmodified cell. The disclosure also provides a biological structure comprising the cell, for example a muscle, a cardiac muscle, an LAA, a RVMS, a heart, and a non-human animal. The non-human animal may be of any taxon. For example, birds and mammals have the advantage of having four-chambered hearts, which can more accurately model the functioning of the human heart. Generally speaking, the more closely related the animal is to humans, the more accurate a model the animal will be. Animals that are closely related to humans (such as other primates) provide accurate models of human heart pathology. However, animals that are less closely related to humans (such as rodents) are easier and less expensive to husband.

### H. Method of Diagnosis and Related Uses

The disclosure provides a method of diagnosing a subject's risk for arrhythmia, comprising measuring a level of expression or activity of the ARC channel, the SOCC, or an activator of the either in a sample from the subject; and comparing the level of expression or activity in the subject to a normal level of expression or activity, wherein an increase in the level of expression or activity indicates an increased risk for arrhythmia. The ARC channel activator or SOCC activator may be any that are described as “target molecules” above, in the context of inhibiting arrhythmia or after-depolarization. Specific embodiments of the method include measuring the subject's level of expression or activity of phospholipase, a ligand of phospholipase, a product of phospholipase, CPLA2, a ligand of CPLA2, a product of CPLA2,

### I. Examples

Experiments were performed to determine whether and what extent the ARC channel influences after-depolarization.

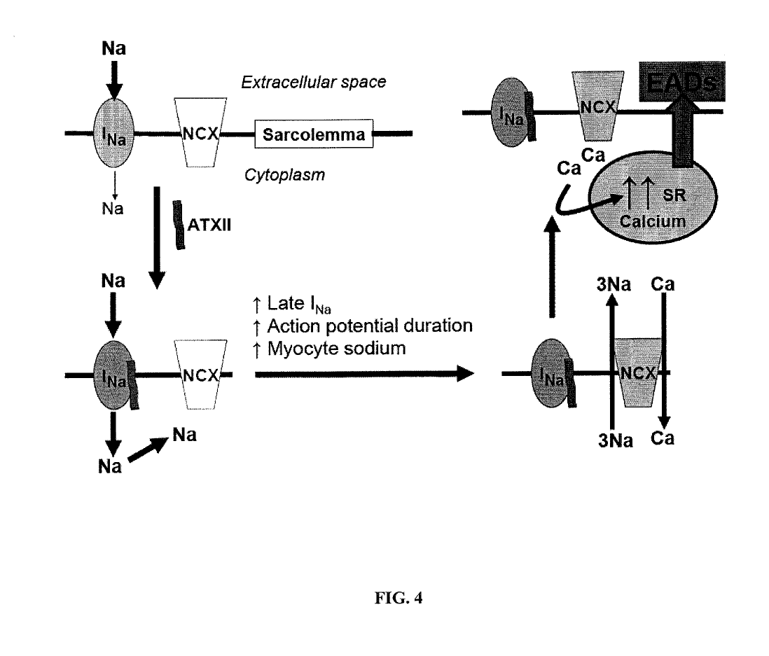

FIG. 4 illustrates the current mechanistic model of after-depolarization. During a normal depolarization the sodium channel (I_{Na}) remains open only briefly and only little sodium enters the cell. After-depolarization can be experimentally induced by treating heart muscle with ATX-II. At low nanomolar concentrations, ATX-II binds to the sodium channel and increases the late sodium current. Under conventional models, three related events follow. First, the action potential duration is prolonged due to the enhanced activity of the depolarizing I_{Na}. Second, increased late I_{Na} increases intracellular sodium concentration and Na⁺-Ca²+ exchanger (NCX) then transports this excess sodium out of cell. Third, sodium exiting via NCX increases intracellular SR calcium concentration, resulting in an early after-depolarization. Hypothetically, SR calcium leak causes early after-depolarization.

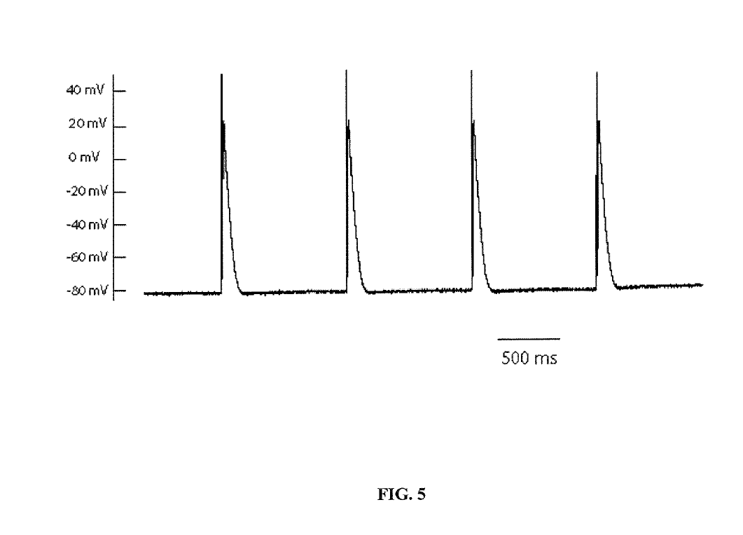

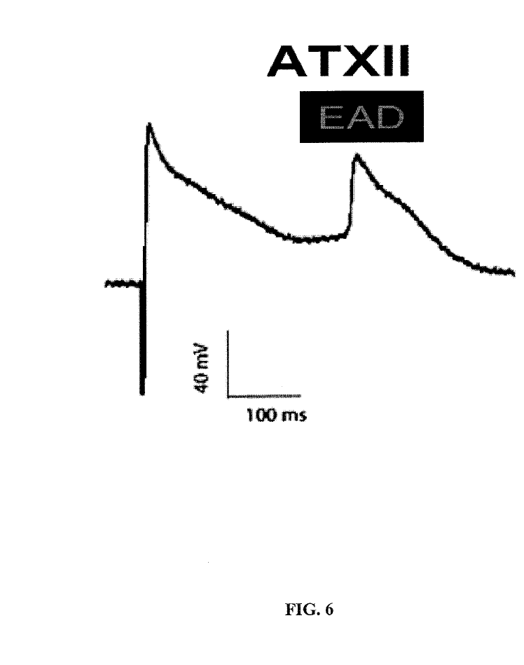

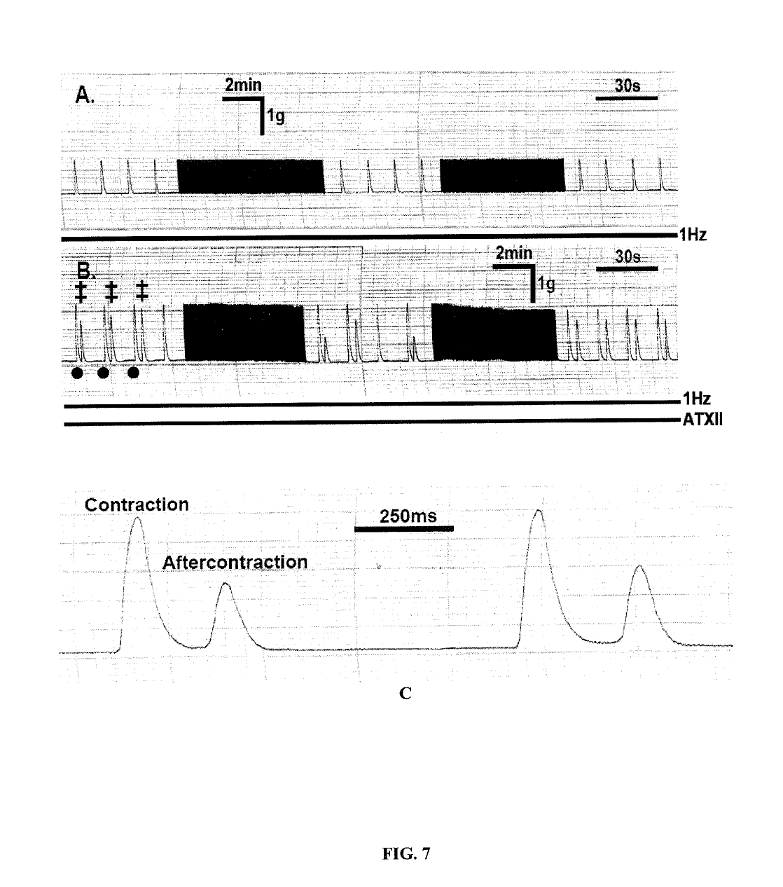

To determine whether ATX-II prolongs action potential duration and provokes after-depolarization and after-contractions, paced isolated rat LAA were superfused at 30° C., impaled with glass microelectrodes, and their action potentials were recorded. ATX-II was then added to the superfusate at about 30 nM and action potentials were recorded again. As shown in FIG. 5, in the absence of ATX-II, the LAA displayed a typical pattern of action potentials. As shown in FIG. 6, exposure to 30 nM ATX-II produced prolonged action potential duration and produced early after-depolarization during repolarization.

FIG. 7 shows the mechanical activity of the LAA when exposed to ATX-II and in its absence. The LAA was externally paced at 1 Hz throughout. FIG. 7A shows that the LAA only contracts in response to pacing when no ATX-II is present. However, as shown in FIG. 7B, in the presence of 30 nM ATX-II, after-contractions are observed (‡) following each paced contraction (•). FIG. 7C shows an enhanced view of the relationship between contractions and after-contractions induced by ATX-II (the time scale is expanded to 50 msec per square).

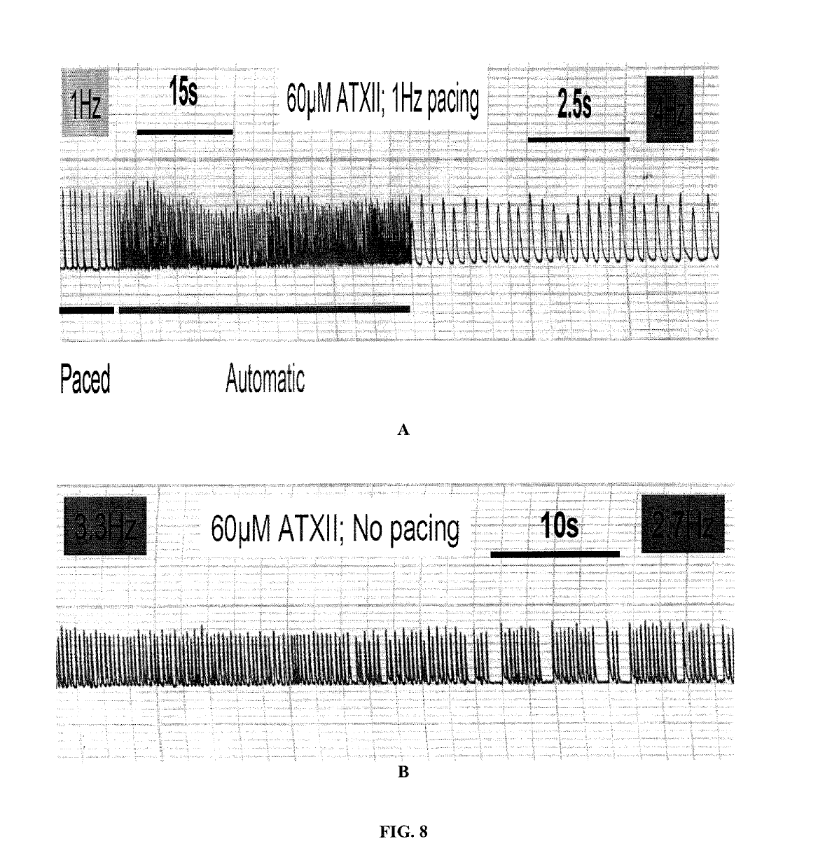

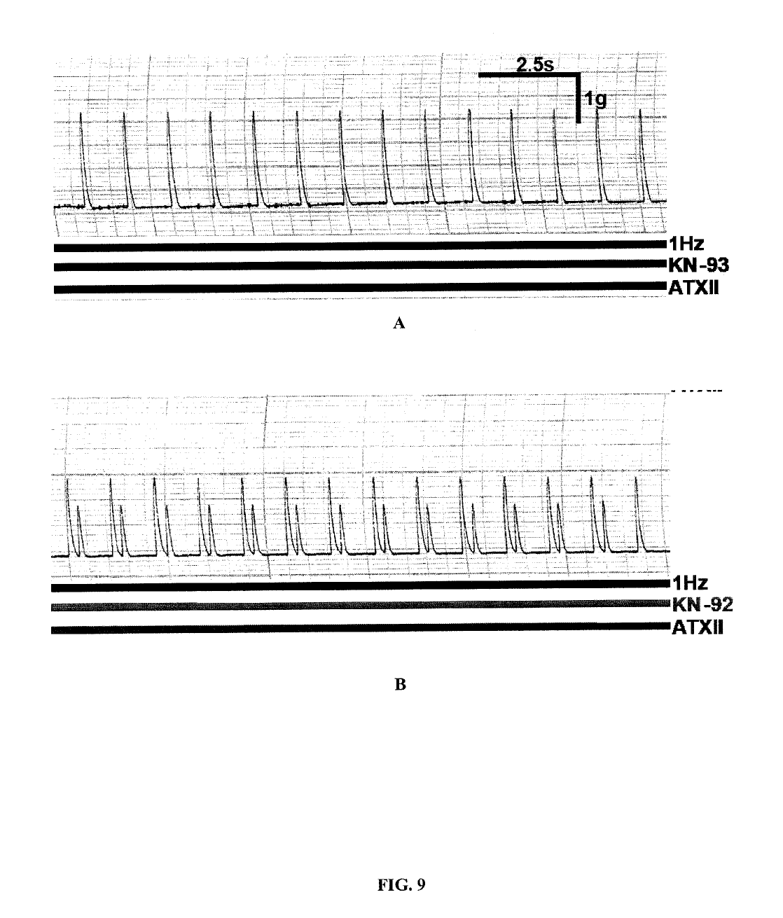

When the experiment was repeated using a higher concentration of ATX-II, the LAA displayed abnormal automatic activity. As shown in FIG. 8A, left atria paced at 1 Hz in the presence of 60 μM ATX-II displayed after-contractions. Subsequently these LAA contracted rapidly (4 Hz) in the absence of pacing, demonstrating automatic activity. As shown in FIG. 8B, this automatic activity persisted in the absence of pacing at 2.7-3.3 Hz.

The effect of inhibitors of calmodulin-dependent protein kinase II (CaMKII) was observed to confirm that after-contractions caused by ATX-II are calcium-dependent. LAA were pre-treated with KN-93, an inhibitor of calmodulin-dependent protein kinase II, and then with ATX-II. As shown in FIG. 9A, such muscles do not produce after-contractions. This result indicates that inhibiting a calcium-dependent protein kinase can prevent left atrial triggered activity. As shown in FIG. 9B, exposing left atria to KN-92 (the inactive congener of KN-93) does not prevent after-contractions in the presence of ATX-II.

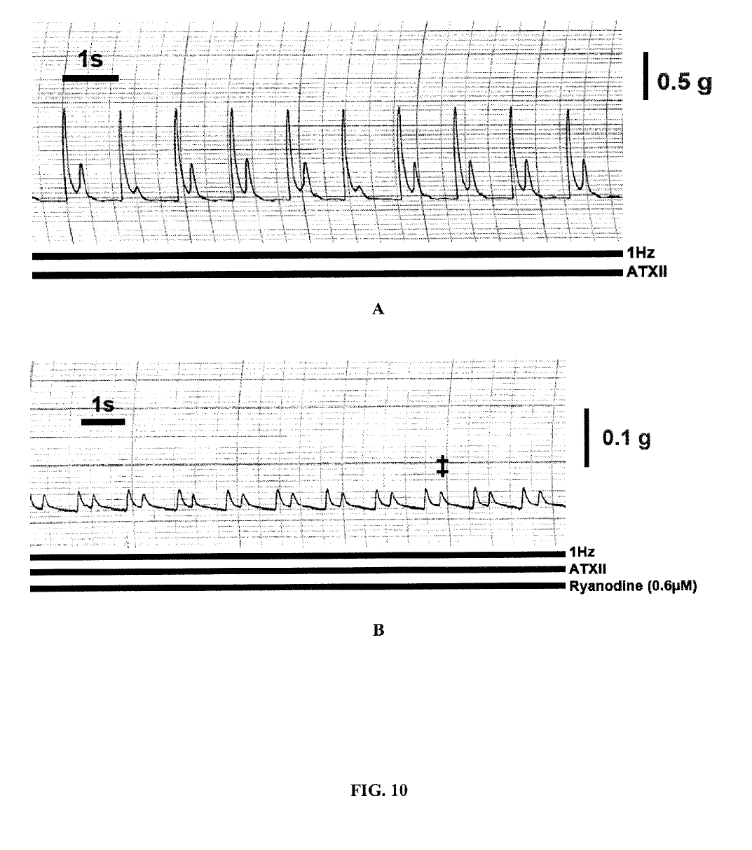

Previously, it was believed that after-depolarization results from the leakage of calcium from the sarcoplasmic reticulum. Transport of calcium from the SR to the cytoplasm is regulated by the ryanodine receptor channel. The ryanodine receptor channel opens in response to exposure to ryanodine, causing all calcium from the SR to enter the cytoplasm. To determine whether after-contractions result from leakage of calcium from the SR, superfused LAA were exposed to ATX-II until a steady-state of after-contractions was achieved (see FIG. 10 A). These muscles were then exposed to 0.6 μM ryanodine. As shown in FIG. 10B, in response to ryanodine exposure the LAA displayed greatly diminished mechanical force, indicating depletion of the SR calcium store. However, after-contractions continued to occur. It was concluded that depletion of SR calcium stores does not affect the production of after-contractions (and by association, after-depolarization).

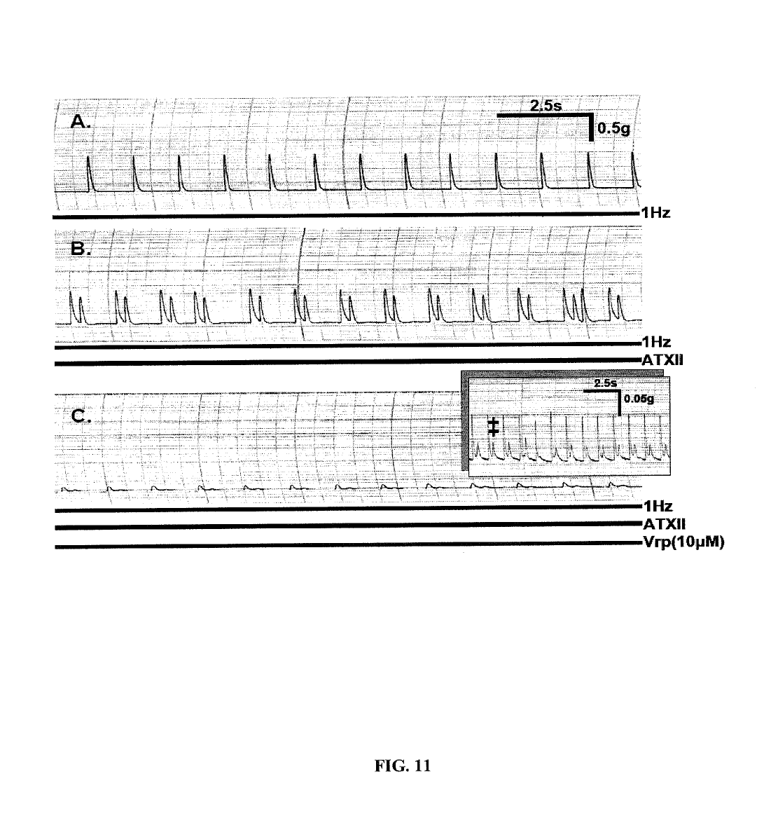

Experiments were performed to determine if the source of calcium causing after-contractions is the voltage-dependent calcium channels by exposing LAA to verapamil, which blocks the entry of calcium into myocytes through voltage-dependent calcium channels. As shown in FIG. 11A, left atria were paced at 1 Hz and displayed normal activity in response. The LAA where then exposed to ATX-II to produce steady state after-contractions (shown in FIG. 11B). As shown in FIG. 11C, exposing these muscles to 10 μM verapamil (Vrp) significantly suppressed mechanical function, but did not affect the production of after-contractions. The suppression of mechanical function occurs because verapamil blocks the entry of calcium into myocytes through voltage-dependent calcium channels. This limits the binding of calcium to the ryanodine receptor and decreases the SR calcium release that precedes contraction. However, it does not appear to block after-contractions.

The sensitivity of ATX-II after-depolarization to CaMKII inhibitors indicates that after-depolarization requires calcium. The inability of ryanodine or verapamil to suppress after-depolarization suggests that the calcium which triggers after-depolarization does not come from the SR ryanodine-sensitive calcium store or from voltage-dependent calcium entry.

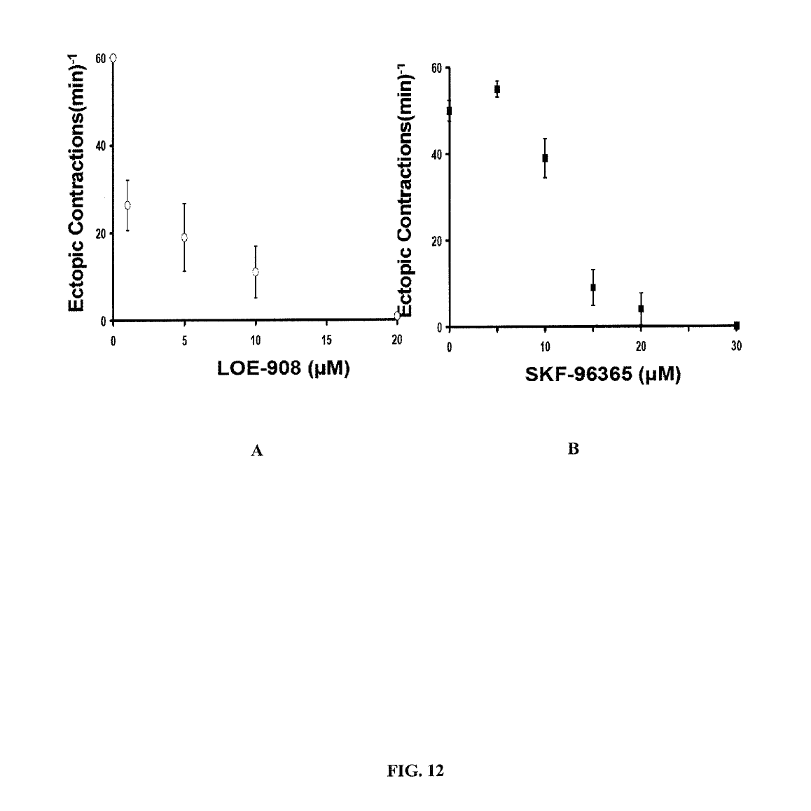

A series of experiments was performed to test the hypothesis that the calcium source for after-depolarization is the ARC channel. To test whether after-contractions and after-depolarization require ARC or SOCC activity, experiments were performed with the ARC channel inhibitor LOE-908 and with the SOCC inhibitor SKF-96365. Rat LAA were superfused at 30° C. and exposed to ˜30 nM ATX-II. After 10-15 minutes, these muscles produced steady-state after-contractions, prolonged action potential duration and after-depolarization as shown previously. As shown in FIGS. 12A and 12B, exposing these muscles to LOE-908 or SKF-96365 suppressed after-contractions in a dose-dependent manner. The same was observed for after-depolarization (data not shown). Importantly, the action potential duration remained prolonged, indicating that ARC activation in conjunction with SOCC activation provokes after-depolarization events and automatic activity.

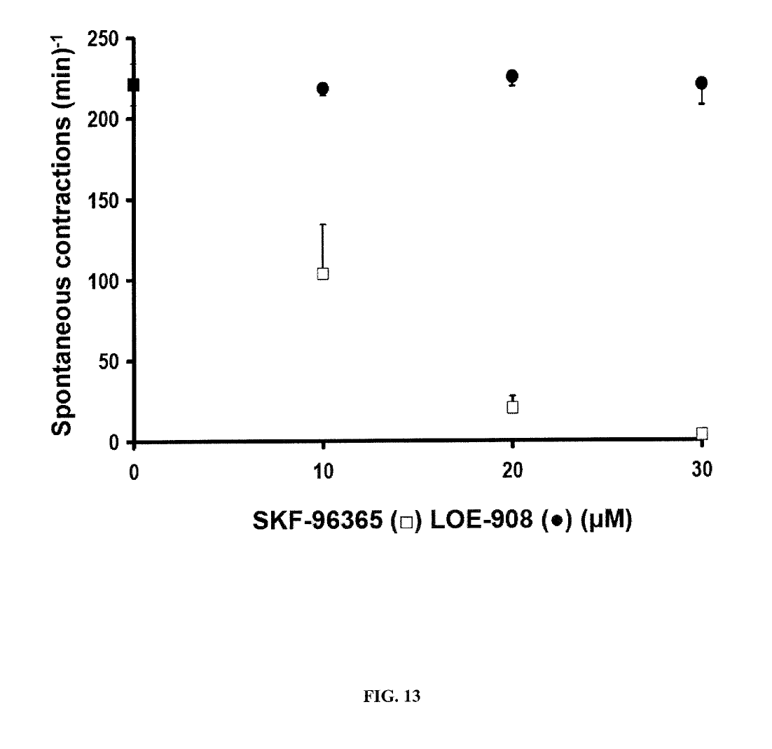

To further test the hypothesis that the calcium source for after-depolarization is not the SOCC, calcium-loaded LAA were treated with 2-aminoethoxy diphenylborate (an activator of the SOCC) to produce abnormal automatic activity. The LAA were then treated with either LOE-908 or SKF-96365. As shown in FIG. 13, SKF-96365 (which suppresses the SOCC) reduces the rate of spontaneous contractions caused by activation of the SOCC, but LOE-908 does not.

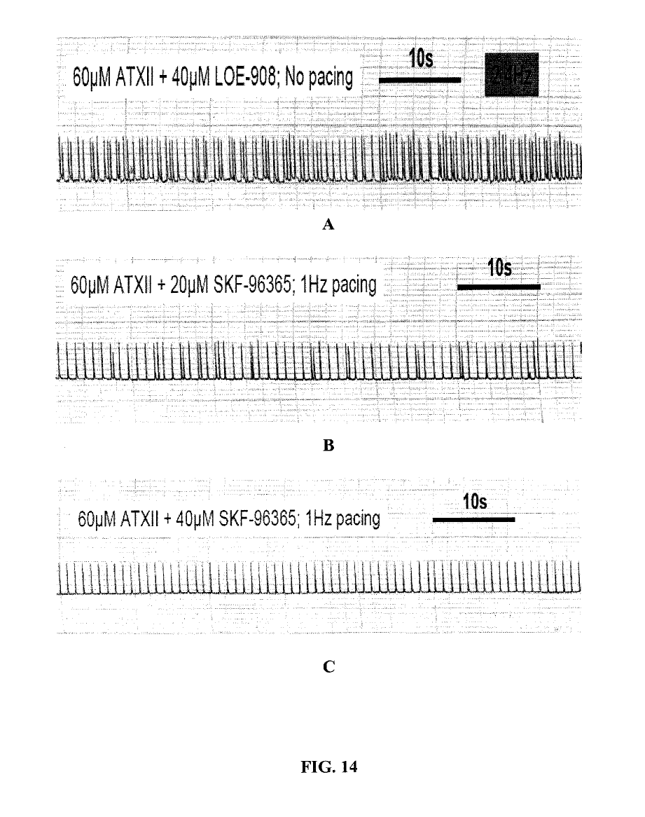

Although moderate concentrations of ATX-II appear to cause after-contractions, high concentrations of ATX-II cause abnormal automatic activity. To further test the hypothesis that after-contractions occur via a separate mechanism from abnormal automatic activity, LAA were exposed to high concentrations (60 nM) of ATX-II. The LAA were then exposed to one of 40 μM LOE-908 without pacing, 20 μM SKF-96365 with 1 Hz pacing, or 40 μM SKF-96365 with 1 Hz pacing. Treatment with LOE-908 failed to suppress abnormal automatic activity (FIG. 14A), whereas treatment with SKF-96365 either reduced or eliminated abnormal automatic activity (FIGS. 14B and 14C).

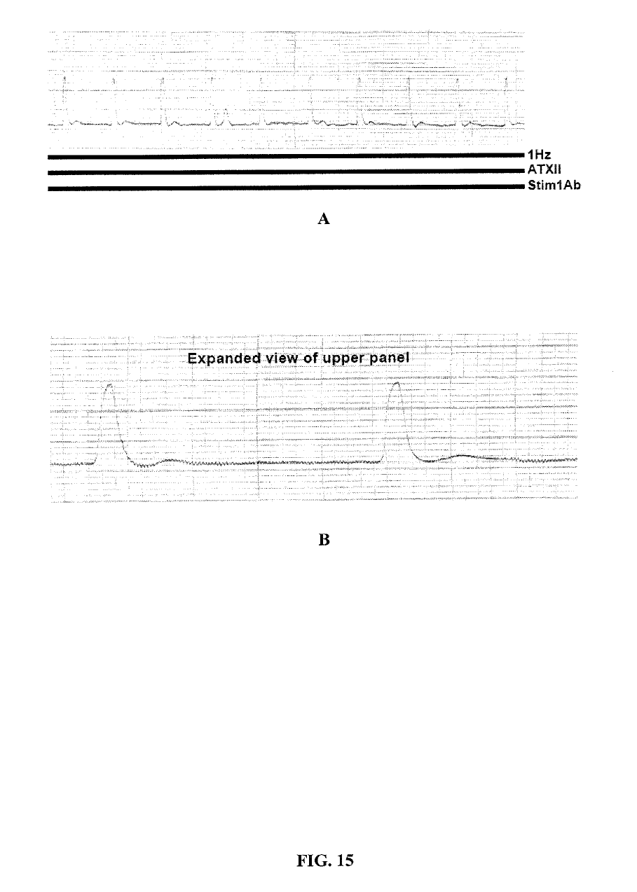

To further test the hypothesis that the ARC channel is the source of calcium for after-contractions, the effects of additional suppressors or ARC channel activity were tested. Antibodies that recognize the N-terminal domain of STIM1 are one such suppressor (Mignen O, Thompson J L, Shuttleworth T J. J Physiology (2006) 579:703-715, incorporated herein only to teach this suppressor of ARC channel activity. Superfused LAA were exposed to 3 μg/mL of the BD Bioscience antibody 610954, which recognizes the N-terminal domain of STIM1. After 45 minutes of exposure to the antibody, the LAA were treated with 30 nM ATX-II and their mechanical function was recorded. As shown in FIGS. 15A and 15B, the LAA showed only low-amplitude after-contractions compared to observations of muscles that had been treated with ATX-II alone (e.g., FIG. 7C).

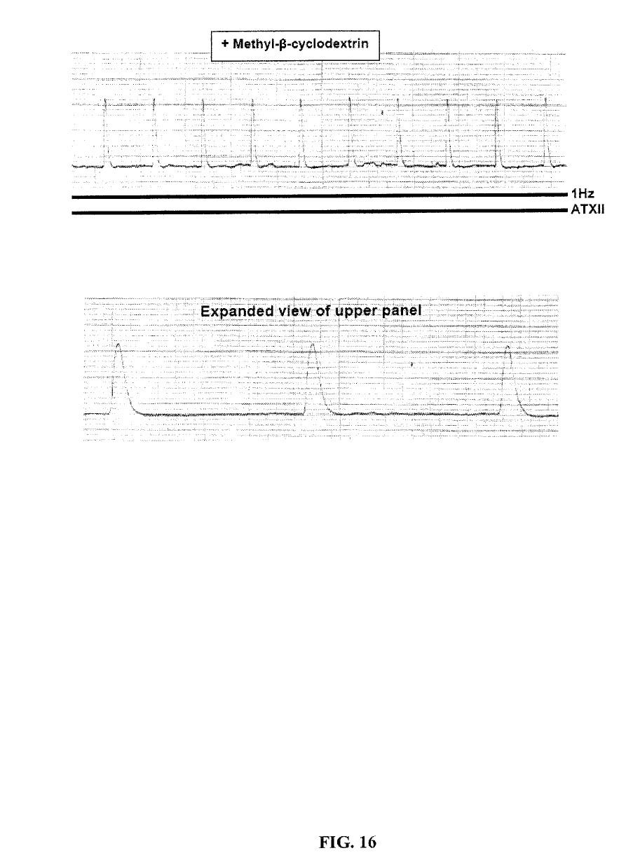

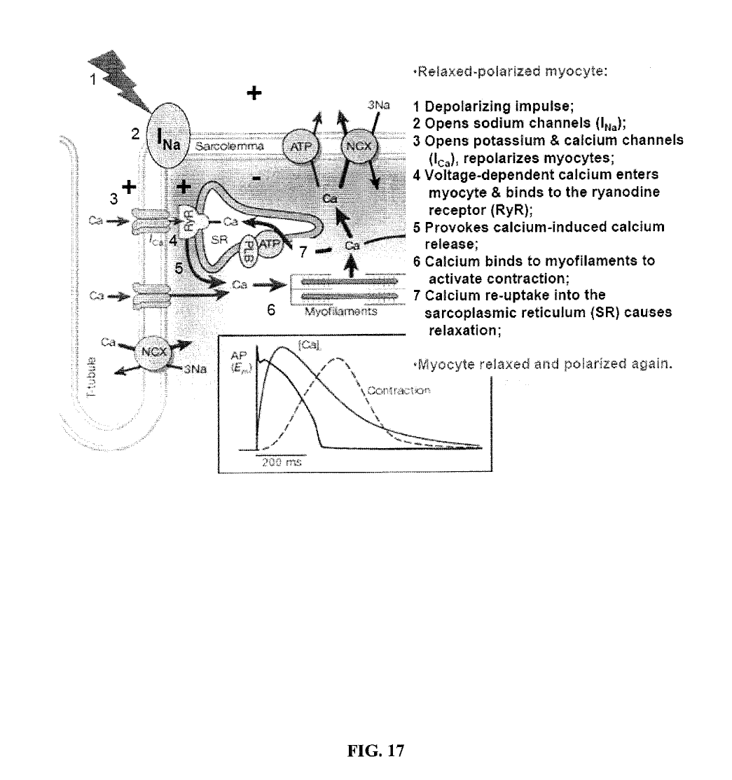

Agents that remove cholesterol from cell plasma membranes can suppress ARC channel activity (Holmes A M, Roderick H L, McDonald F, Bootman M D, Cell Calcium (2007) 41:1-12, which is incorporated herein only to teach such agents as suppressors of ARC channel activity). One such agent is methyl-β-cyclodextrin (MβC), which has been observed to extract cholesterol from the plasma membane (Rodal et al., Mol. Biol. of the Cell, (1999) 10:961-974, which is incorporated herein by reference to teach the extraction of cholesterol from the plasma membrane using this compound). LAA were exposed to MβC to determine its effect on after-contractions. LAA were superfused for 30 mM with 10 mM MβC and then washed extensively to remove this cholesterol chelator. LAA then were exposed to ˜30 nM ATX-II and mechanical function was recorded over a 20 min period. Muscles treated with MβC produced no after-contractions following their exposure to ATX-II (FIGS. 16A and 16B). This is further evidence that after-depolarization requires ARC channel activity.

In a separate experiment, isolated rat cardiac muscle was incubated with ATX-II in the presence of varying concentrations of amylcinnamoyl-anthranilic acid. Fig. X shows that incubation with 0 μM ACA did not inhibit after-depolarization. The frequency of aftercontractions in the muscle decrease with increasing concentration of ACA in a range from 0-50 μM of ACA. The calculated IC₅₀ for ACA was 10±4.5 μM. In a similar experiment it was determined that the IC₅₀ of aristolochic acid is 31±5 μM.

### I. Conclusions

The foregoing description illustrates and describes the processes, machines, manufactures, compositions of matter, and other teachings of the present disclosure. Additionally, the disclosure shows and describes only certain embodiments of the processes, machines, manufactures, compositions of matter, and other teachings disclosed, but it is to be understood that the teachings of the present disclosure are capable of use in various other combinations, modifications, and environments and are capable of changes or modifications within the scope of the teachings as expressed herein, commensurate with the skill and knowledge of a person having ordinary skill in the relevant art. The embodiments described are further intended to explain certain best modes known of practicing the processes, machines, manufactures, compositions of matter, and other teachings of the present disclosure and to enable others skilled in the art to utilize the teachings of the present disclosure in such, or other, embodiments and with the various modifications required by the particular applications or uses. Accordingly, the processes, machines, manufactures, compositions of matter, and other teachings of the present disclosure are not intended to limit the exact embodiments and examples disclosed herein

1-7. (canceled)

8. A method of preventing or reversing an after-depolarization in a myocyte comprising contacting the myocyte with an inhibitor of the ARC channel, an inhibitor of a molecule that stimulates ARC channel activity, an inhibitor of the SOCC, or an inhibitor of a molecule that stimulates SOCC activity.

9. The method of claim 8, wherein the inhibitor is selected from the group consisting of an inhibitor of a phospholipase, an inhibitor of cellular voltage-independent calcium homeostasis, and an inhibitor of a voltage-independent calcium channel.

10. The method of claim 9, wherein the inhibitor is an inhibitor of Stim1.

11. The method of claim 9, wherein the voltage-independent calcium channel is a calcium channel regulated by arachidonic acid, regulated by linoleic acid, regulated by a metabolite of arachidonic acid, regulated a metabolite of linoleic acid, or regulated by intracellular calcium stores.

12. The method of claim 9, wherein the phospholipase is activated by an increase in intracellular calcium.

13. A pharmaceutical composition for at least one of the treatment or prevention of cardiac arrhythmia, the composition comprising an inhibitor of the ARC channel, an inhibitor of a molecule that stimulates ARC channel activity, an inhibitor of the SOCC, or an inhibitor of a molecule that stimulates SOCC activity.

14. The pharmaceutical composition of claim 13, wherein the inhibitor is selected from the group consisting of an inhibitor of a phospholipase, and an inhibitor of a voltage-independent calcium channel.

15. The pharmaceutical composition of claim 13, Wherein the inhibitor is an inhibitor of Stim1.

16. The pharmaceutical composition of claim 15, wherein the voltage-independent calcium channel is a calcium channel regulated by arachidonic acid, regulated by linoleic acid, regulated by a metabolite of arachidonic acid, regulated a metabolite of linoleic acid, or regulated by intracellular calcium stores.,

17. The pharmaceutical composition of claim 15, wherein the phospholipase is activated by an increase in intracellular calcium.

18. A method of treating or preventing a cardiac arrhythmia in a subject in need thereof, comprising administering to the subject the pharmaceutical of claims 13.

19. An assay for detecting an anti-arrhythmia agent comprising contacting a candidate substance to a target that is the ARC channel, a molecule that stimulates ARC channel activity, the SOCC, or a molecule that stimulates SOCC activity, and measuring at least one of (1) the binding of the candidate to the target, and (2) the activity of the target.

20. The assay of claim 19, wherein the target is selected from the group consisting of: phospholipase, a phospholipase ligand, a voltage-independent calcium channel, and the SOCC.

21. An assay for the identification of an anti arrhythmic agent comprising:
(a) exposing a myocyte to an arrhythmic agent at an arrhythmia-inducing effective concentration, wherein the arrhythmic agent induces an after depolarization in the myocyte;
(b) exposing the myocyte to a candidate anti-arrhythmic agent before, after, or simultaneously with exposing the myocyte to the arrhythmic agent;
(c) obtaining a first measurement of a parameter indicative of arrhythmia in the myocyte in the presence of the arrhythmic agent in the absence of the candidate;
(d) obtaining a second measure of the parameter in the presence of the arrhythmic agent and in the presence the candidate anti-arrhythmic agent; and
(d) comparing the first measurement to the second measurement, wherein an improvement in the parameter indicates the identification of an anti-arrhythmic agent.

22. The assay of claim 20, wherein the myocyte is an isolated myocyte, a component of a cardiac muscle, or a component of an intact heart.

23. The assay of claim 21, wherein the cardiac muscle is selected from the group consisting of: a perfused heart, a portion of a perfused heart, a portion of a heart, a left atrial appendage, a ventricular muscle strip and a right ventricular muscle strip.

24. The assay of claim 20, further comprising increasing the concentration of calcium, arachidonate, linoleic acid, a metabolite of arachidonic acid, or a metabolite of linoleic acid in the myocyte.

25. The assay of claim 20, wherein the arrhythmic agent leads to the activation of a voltage-independent calcium channel.

26. The assay of claim 24, wherein the voltage-independent calcium channel is selected from the group consisting of the ARC channel and the SOCC.

27. The assay of claim 24, wherein the arrhythmic agent is selected from the group consisting of ATX-II, a physiologically acceptable salt of ATXII, and a genetic modification that results in increased ARC channel activity.

28. A method of diagnosing a subject's risk, for arrhythmia, said method comprising: measuring a level of expression or activity of the ARC channel, a molecule that stimulates ARC channel activity, the SOCC, or a molecule that stimulates SOCC activity in a sample from the subject; and comparing the level of expression or activity in the subject to a normal level of expression or activity, wherein an increase in the level of expression or activity indicates an increased risk for arrhythmia.

29. The method of claim 28, wherein the molecule that stimulates the ARC channel is a phospholipase.

30. The method of claim 28, wherein the molecule that stimulates ARC activity is a phospholipase ligand or a phospholipase product.

31. (canceled)
